(19) United States
(12) Patent Application Publication
(10) Pub. No.: US 2025/0282881 A1
(43) Pub. Date: Sep. 11, 2025

(54) ANTIBODIES AND BISPECIFIC BINDING PROTEINS THAT BIND OX40 AND/OR PD-L1

(71) Applicant: SHANGHAI EPIMAB BIOTHERAPEUTICS CO., LTD., Shanghai (CN)

(72) Inventors: Shiyong GONG, Shanghai (CN); Baocun LI, Shanghai (CN); Fan LIU, Shanghai (CN); Chengbin WU, Shanghai (CN); Xuan WU, Shanghai (CN); Rui ZHANG, Shanghai (CN)

(21) Appl. No.: 18/568,252

(22) PCT Filed: Jun. 9, 2022
(86) PCT No.: PCT/CN2022/097889

§ 371 Date: Dec. 7, 2023

(30) Foreign Application Priority Data
Jun. 9, 2021 (WO) PCT/CN2021/099228

(51) Int. Cl. C07K 16/28; A61K 39/00; A61P 35/00; G01N 33/53

(52) U.S. Cl. CPC C07K 16/2878; A61P 35/00; G01N 33/53; A61K 2039/505; C07K 2317/24; C07K 2317/31; C07K 2317/522; C07K 2317/565; C07K 2317/76; C07K 2317/92; G01N 2333/70578

(57) ABSTRACT

Provided are new antibodies recognizing TNF receptor superfamily member OX40, new antibodies recognizing Programmed Death-Ligand 1 (PD-L1), and bispecific OX40/PD-L1 binding proteins such as FIT-Ig binding proteins made using those antibodies. The antibodies and bispecific binding proteins are useful for treatment of diseases such as cancers.

### CROSS-REFERENCE TO RELATED APPLICATIONS

This application is a U.S. National Stage Application under U.S.C. § 371 of International Application No. PCT/CN2022/097889 filed Jun. 9, 2022, which claims priority of International Application No. PCT/CN2021/099228 filed Jun. 9, 2021, the contents of each of which are hereby incorporated by reference in their entireties for all purposes.

### SEQUENCE LISTING

The instant application contains a Sequence Listing which has been submitted in ASCII format via Patent Center and is hereby incorporated by reference in its entirety. Said ASCII copy, created on Feb. 14, 2024, is named 379446_ST25.txt and is 48,467 bytes in size.

### TECHNICAL FIELD

The present disclosure relates to antibodies capable of recognizing tumor necrosis factor receptor OX40 (CD134), and related bispecific binding proteins comprising at least one OX40 binding domain and at least one PD-L1 binding domain, such as bispecific OX40/PD-L1 binding proteins (e.g., Fabs-in-Tandem immunoglobulin (FIT-Ig) binding proteins). The present disclosure also relates to antibodies capable of recognizing PD-L1 and related bispecific binding proteins comprising at least one PD-L1 binding domain and at least one OX40 binding domain, such as bispecific OX40/PD-L1 binding proteins (e.g., FIT-Ig binding proteins). The antibodies and bispecific binding proteins disclosed herein may be useful for disease treatment, for instance, in cancer immunotherapy. The present disclosure further relates to a nucleic acid encoding said antibody or bispecific binding protein, and a method of producing said antibody or bispecific binding protein.

### BACKGROUND

The tumor necrosis factor (TNF) receptor superfamily (TNFR) is a large class of functionally diverse receptors capable of mediating a range of immune cell function (Mayes P A, 2018). Many members of the TNFR superfamily are co-stimulatory receptors which can be expressed on a number of immune cell types, including T cells, B cells and natural killer (NK) cells, as well as antigen-presenting cells (APCs), and have been shown to induce immune cell function, proliferation and survival (Watts T. H., 2005).

OX40 (CD134), a member of the TNFR superfamily with type I transmembrane glycoprotein characterized by 4 cysteine-rich domains (CRDs), is expressed mostly on activated CD4 and CD8 T cells and Foxp³⁺CD⁴⁺ regulatory T cells (Treg), while its ligand, OX40L (CD252), is expressed on activated APCs, for example, dendritic cells (DCs), B cells and macrophages (Weinberg A D, 2011). Upon activation by TCR-MHC/peptide interaction, OX40L homotrimer is formed and binds to three OX40 receptors to result in receptor crosslinking (Watts, 2005; Jane Willoughby, 2017). The higher order super-clustering of OX40 was suggested to be necessary for mediating downstream signaling. The clustered OX40 receptors recruit TNF receptor associated factors (TRAF) to the intracellular domain of OX40. TRAF2 and 3 activate PI3K/PKB, nuclear factor κB1 (NF-κB1) and NFAT pathways that account for T cell division, survival and cytokine production (Croft, 2010; Kawamata, 1998; Song, 2008). Thus, signaling downstream of OX40 has the potential to augment proliferation, suppress apoptosis and induce greater cytokine response from T cells, all of which are functional outcomes that conferring OX40 agonistic antibodies the capacity to elicit when used in immunotherapy.

The mechanism of agonistic anti-OX40 antibodies in mediating anti-tumor efficacy have been investigated extensively in various mouse tumor models. Most agonistic anti-OX40 mAbs adopt human IgG1 isotype for strong FcγR binding to trigger the co-stimulating signaling pathways on effector T cells, thereby supporting the survival and expansion of activated T cell subsets and the establishment of T cell memory of CD8 T cell responses by OX40 (Brendan D Curti, 2013; Glisson, 2020). Additional data suggest that OX40 costimulation inhibits the FoxP3 expression and Treg induction via downstream signaling (Zhang X, 2018). Since OX40 is highly expressed on infiltrated Tregs, induction of anti-tumor responses by OX40 antibodies relies on depletion of intra-tumor Treg cells through Fc-mediated effector functions by antibody-dependent cellular cytotoxicity (ADCC) and antibody-dependent cellular phagocytosis (ADCP) (Aspeslagh, 2016; Smyth, 2014). However, depletion of intra-tumoral Tregs could improve the ratio of infiltration of CD8 effector T cells to Tregs in tumor microenvironment (TME), as demonstrated by enhanced anti-tumor immune response and improved survival in several mouse models (Jacquemin, 2015; Bulliard, 2014). Owing to their favorable anti-tumor efficacy, most agonistic OX40 antibodies under clinical development are of IgG1 isotype for their desirable anti-tumor efficacy (Choi, 2020; Brendan D Curti, 2013; Glisson, 2020). Clinical trials using OX40-targeted drugs have illustrated its safety when used as monotherapy or in combination with immune check blockers (ICB). Although OX40-targeted therapy has demonstrated impressive results in preclinical mouse models, its efficacy as monotherapy in humans is modest according to preliminary clinical data (Glisson, 2020; Carolina, 2020; Martin Gutierrez, 2020). Nevertheless, OX40 co-stimulation in combination with either anti-PD-1, anti-PD-L1, or anti-CTLA4 did not produce clear improvement in efficacy according to a recently published phase ½a study (Martin Gutierrez, 2020). The low response to agonistic anti-OX40 antibodies in a patient may be attributable to two causes. First, FcγR-dependent clustering in some tumors would be less efficient when limited by available infiltrated FcγRs in TME (Willoughby, 2017), or in the presence of high concentration of endogenous IgG which competes for FcγR binding (Christian Gieffers, 2013). Second, as found by a recent clinical study, the percentage of OX40 CD4 memory T cells was reduced after an anti-OX40 IgG1 antibody treatment, possibly due to antibody dependent cellular cytotoxicity (ADCC) and antibody dependent cellular phagocytosis (ADCP) of OX40⁺ cells (Glisson, 2020). Thus, there is a need for a new generation of anti-OX40 agonists that could mediate effective super-clustering, regardless of the limited availability of FcγR, while maintaining low effector function.

PD-L1 (CD274) is a 40 kDa type I transmembrane protein, the PD-1/PD-L1 signaling pathway plays an important role in immune tolerance and tumor immune evasion. PD-L1 is expressed in many human tumor tissues (e.g., lung cancer, gastric cancer, breast cancer, and intestinal cancer). Blocking the PD-1/PD-L1 inhibitory signaling pathway activates suppressed T cells to attack cancer cells. Most anti-PD-L1 mAbs inhibit tumor growth both in vivo and in patient by promoting proliferation of tumor antigen specific T cells (Julie, 2012; Brahmer, 2012). Bispecific antibodies are a class of engineered antibodies having dual-affinity against two different antigens/epitopes. Bispecific antibodies in various forms have been reported and explored, including the FIT-IG (Fabs-In-Tandem ImmunoGlobulin) as disclosed in WO2015/103072.

### SUMMARY OF INVENTION

The present disclosure provides new antibodies that bind to PD-L1 and new antibodies bind to OX40 with high affinity. The present disclosure also provides PD-L1/OX40 bispecific Fabs-in-Tandem immunoglobulins (FIT-Igs) that simultaneously bind both PD-L1 and OX40. Antibodies and bispecific binding proteins of the present disclosure can block PD-L1 inhibitory signaling on tumor infiltrating lymphocytes (TILs) to reactivate tumor infiltrated cyotoxic T cells to tumor cells.

The bispecific antibody of the present disclosure comprises two antigen-binding regions have dual mechanism, firstly, through its PD-L1-binding region, the bispecific antibody agent binds to PD-L1 expressing tumor cells or APCs, while through its OX40 binding region, the bispecific antibody could bind OX40 and mediate super-clustering, therefore activate T cells in a conditional PD-L1 dependent manner. Second, the bispecific antibody of the present disclosure blocks the binding of human PD-L1 to human PD-1 to preventing PD-L1 mediating immunity evading though PD-1. Thus, the bispecific antibody of the present disclosure activates T cells through binding to OX40, while preventing T cell exhaustion through PD-1/PD-L1 interaction, in turn, results in strengthened T cell activation, proliferation of effector and memory to boost anti-tumor efficacy. Moreover, the bispecific antibody of the present disclosure introduces LALA mutation in the Fc region to attenuate ADCC and ADCP to the OX40 positive T cells.

The disclosed PD-L1/OX40 bispecific antibody overcomes the limitation of an anti-OX40 monotherapy by inducing high-order OX40 clustering and triggering sufficient OX40 signaling through PD-L1 crosslinking. The simultaneous binding of PD-L1 on tumor cells and OX40 on T cells, results in both PD-L1-dependent activation of OX40 on T cells along with inhibition of PD-1/PD-L1 inhibitory signaling, which may lead to efficient induction of anti-tumor immunity. Therefore, OX40/PD-L1 bispecific antibodies have utility in the treatment of cancers.

### BRIEF DESCRIPTION OF THE DRAWINGS

FIG. 1 shows epitope identification of anti-OX40 antibodies. FIG. 1a shows the binding of HuEM1007-044-16 (top), OX40-Tab1 (middle), OX40-Tab2 (bottom) to full length of extracellular OX40 (CRD1-4, circle) and truncated OX40 variants ΔCRD1 (lacking CRD1, square), ΔCRD1-2 (lacking CRD1 and CRD2, triangle), ΔCRD1-3 (lacking CRD1, CRD2 and CRD3, diamond). FIG. 1b shows the binding of OX40-Tab2 to full length of extracellular OX40 (CRD1-4, circle), mCRD1 (CRD1-4 with CRD1 domain therein replaced by murine CRD1, square), mCRD2 (CRD1-4 with CRD2 domain therein replaced by murine CRD2, triangle), mCRD3 (CRD1-4 with CRD3 domain therein replaced by murine CRD3, inverted triangle), and mCRD4 (CRD1-4 with CRD4 domain therein replaced by murine CRD4, diamond).

FIG. 2 shows anti-OX40 antibody HuEM1007-044-16 (black) induced selective proliferation of T effector cells over Treg cells. Irrelevant human IgG (gray) was used for a negative control.

FIG. 3 illustrates CHO-PD-L1 binding of serially diluted antibodies FIT1014-20a (diamond) and HuEM0005-86-64 (square), together with irrelevant human IgG (triangle) as a negative control, as measured by FACS.

FIG. 4 indicates the result of FACS affinity for binding to human OX40-transfected CHO cell, involving serially diluted antibodies FIT1014-20a (diamond) and its parental OX40 antibody HuEM1007-44-16 (square). Irrelevant human IgG (triangle) is used for a negative control.

FIG. 5 illustrates the blocking of PD-1/PD-L1 binding by bispecific FIT1014-20a (square) and the parental PD-L1 antibody (triangle) as well as irrelevant human IgG (inverted triangle) as a negative control in a cell-based receptor blocking assay.

FIG. 6 illustrates the blocking of PD-L1 mediated inhibitory signaling by bispecific FIT1014-20a (square) and the parental PD-L1 antibody (triangle) as well as irrelevant human IgG (inverted triangle) as a negative control.

FIG. 7 (top) displays activation of OX40 downstream signaling by bispecific FIT1014-20a (square) and the combination of two parental antibodies comprising identical PD-L1 and OX40 binding domains respectively (inverted triangle), as well as irrelevant human IgG as a negative control (diamond). In a control assay (bottom) CHO cells not expressing. PD-L1 show a lack of activation by FIT1014-20a or a combination of the two parental antibodies.

FIG. 8 shows IL2 (top, 72 hours post incubation) and IFN-γ (bottom, 48 hours post incubation) production from co-culture system of CHO-PD-L1-OS8 cells and human primary T cells upon co-incubation with FIT1014-20a (square), the combination of parental antibodies (inverted triangle), or irrelevant human IgG (diamond).

FIG. 9 displays T cell activation assessed by IL2 level observed from mixed lymphocyte reaction (MLR) assay after 3 days of incubation with FIT1014-20a (dark) and combination of parental antibodies (gray)

FIG. 10 shows T cell activation assessed by IL2 level observed from Staphylococcus aureus enterotoxin B (SEB) assay after 96 hours of incubation with FIT1014-20a (square), the combination of parental antibodies (inverted triangle), or an irrelevant human IgG as negative control (diamond).

FIG. 11 shows complement dependent cytotoxicity assay of FIT1014-20a (circle), with anti HLA-1 as positive control (triangle), and irrelevant human IgG as negative control (square).

FIG. 12 shows phagocytosis effect to CHO-OX40 by FIT1014-20a (solid black), HuEM1007-044-16-hIgG1 (solid gray), HuEM1007-044-16 (diagonal stripes), OX40-Tab2 (horizontal stripes), and irrelevant hIgG (checker board).

FIG. 13 shows the assessment of anti-tumor efficacy in humanized OX40 and PD-L1 B6 mice bearing MC38-hPD-L1 tumor cells treated with FIT1014-20a (triangle), parental PD-L1 mAb HuEM0005-86-64 (square), Atezolizumab (square), and vehicle as negative control (circle).

FIG. 14 shows the tumor volume profile of CT26-hPD-L1 syngeneic tumors established in human PD-1/PD-L1/OX40 knock-in mice treated with vehicle control (circle), reference PD-L1 antibody Atezolizumab (square) and FIT1014-20a (triangle). Arrows indicate administration of assigned agent.

### DETAILED DESCRIPTION

This present disclosure pertains to anti-OX40 antibodies, anti-PD-L1 antibodies, antigen-binding portions thereof, and multivalent, bispecific binding proteins such as FIT-Igs that bind to both OX40 and PD-L1. Various aspects of the present disclosure relate to anti-OX40 and antigen binding fragments thereof, anti-PD-L1 antibodies and antigen binding fragments thereof, FIT-Ig binding proteins that bind to human OX40 and human PD-L1, and pharmaceutical compositions thereof, as well as nucleic acids, recombinant expression vectors and host cells for making such antibodies, antigen binding fragments, and binding proteins. Methods of using the antibodies, antigen binding fragments, and bispecific binding proteins of the present disclosure to detect human OX40, human PD-L1, or both; to modulate human OX40 and/or human PD-L1 activity, either in vitro or in vivo; to induce and/or enhance adaptive immune responses against foreign antigens such as, for example, tumors; and to treat diseases, especially cancer, are also encompassed by the present disclosure.

### Definitions

Unless otherwise defined herein, scientific and technical terms used in connection with the present disclosure shall have the meanings that are commonly understood by those of ordinary skill in the art. In the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991) and is referred to as “numbering according to Kabat” herein. Specifically, the Kabat numbering system described by Kabat, et al., Sequences of Proteins of Immunological Interest, 5th ed., Public Health Service, National Institutes of Health, Bethesda, MD (1991) (see pages 647-660) is used for the light chain constant domain CL of kappa and lambda isotype, and the Kabat EU index numbering system (see pages 661-723) is used for the constant heavy chain domains (CH1, Hinge, CH2 and CH3, which is herein further clarified by referring to “numbering according to Kabat EU index” in this case).

The term “isolated protein” or “isolated polypeptide” is a protein or polypeptide that by virtue of its origin or source of derivation is not associated with naturally associated components that accompany it in its native state, is substantially free of other proteins from the same species, is expressed by a cell from a different species, or does not occur in nature. A polypeptide that is chemically synthesized or synthesized in a cellular system different from the cell from which it naturally originates may be “isolated” from its naturally associated components. A protein may also be rendered substantially free of naturally associated components by isolation, using protein purification techniques well known in the art.

The term “specific binding” or “specifically binding” in reference to the interaction of an antibody, a binding protein, or a peptide with a second chemical species, means that the interaction is dependent upon the presence of a particular structure (e.g., an antigenic determinant or epitope) on the second chemical species. For example, an antibody recognizes and binds to a specific protein structure rather than to proteins generally. In general, if an antibody is specific for epitope “A”, the presence of a molecule containing epitope A (or free, unlabeled A), in a reaction containing labeled “A” and the antibody, will reduce the amount of labeled A bound to the antibody. In accordance with the present disclosure, the specific binding protein binds to the corresponding antigen with a K_{D} of 10 nM or lower, for instance, 1 nM or lower. The term “K_{D}” refers to the equilibrium dissociation constant (the reciprocal of the equilibrium binding constant) and is used herein according to the definitions provided in the art. The K_{D} value with which the antibody or binding protein binds to corresponding antigen can be determined by well-known methods including, but not limited to, fluorescence titration, competition ELISA, calorimetric methods, such as isothermal titration calorimetry (ITC), flow cytometric titration analysis (FACS titration), Bio-Layer Interferometry (BLI) and surface plasmon resonance (BIAcore).

The term “antibody” broadly refers to any immunoglobulin (Ig) molecule comprised of four polypeptide chains, two heavy (H) chains and two light (L) chains, or any antigen binding fragment, mutant, variant, or derivation thereof, which retains the essential epitope binding features of an Ig molecule. Such mutant, variant, or derivative antibody formats are known in the art and non-limiting embodiments are discussed below.

In a full-length antibody, each heavy chain is comprised of a heavy chain variable region (abbreviated herein as VH) and a heavy chain constant region. The heavy chain constant region is comprised of three domains: CH1, CH2, and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FRs). Each VH and VL is comprised of three CDRs and four FRs, arranged from amino-terminus to carboxy-terminus in the following order: FR1, CDR1, FR2, CDR2, FR3, CDR3, FR4. First, second and third CDRs of a VH domain are commonly enumerated as CDR-H1, CDR-H2, and CDR-H3; likewise, first, second and third CDRs of a VL domain are commonly enumerated as CDR-L1, CDR-L2, and CDR-L3. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “Fc region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, i.e., a CH2 domain and a CH3 domain, and optionally comprises a CH4 domain, for example, as in the case of the Fc regions of IgM and IgE antibodies. The Fc region of IgG, IgA, and IgD antibodies comprises a hinge region, a CH2 domain, and a CH3 domain. In contrast, the Fc region of IgM and IgE antibodies lacks a hinge region but comprises a CH2 domain, a CH3 domain and a CH4 domain. Variant Fc regions having replacements of amino acid residues in the Fc portion to alter antibody effector function are known in the art (see, e.g., Winter et al., U.S. Pat. Nos. 5,648,260 and 5,624,821). The Fc portion of an antibody may mediate one or more effector functions, for example, cytokine induction, ADCC, phagocytosis, complement dependent cytotoxicity (CDC), and/or half-life/clearance rate of antibody and antigen-antibody complexes. In some cases, these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to FcγRs and complement C1q, respectively. In still another embodiment at least one amino acid residue is replaced in the constant region of the antibody, for example the Fc region of the antibody, such that effector functions of the antibody are altered. The dimerization of two identical heavy chains of an immunoglobulin is mediated by the dimerization of CH3 domains and is stabilized by the disulfide bonds within the hinge region that connects CH1 constant domains to the Fc constant domains (e.g., CH2 and CH3). The anti-inflammatory activity of IgG is dependent on sialylation of the N-linked glycan of the IgG Fc fragment. The precise glycan requirements for anti-inflammatory activity have been determined, such that an appropriate IgG1 Fc fragment can be created, thereby generating a fully recombinant, sialylated IgG1 Fc with greatly enhanced potency (see, Anthony et al., Science, 320:373-376 (2008)).

The terms “antigen-binding portion” and “antigen-binding fragment” or “functional fragment” of an antibody are used interchangeably and refer to one or more fragments of an antibody that retain the ability to specifically bind to an antigen, i.e., the same antigen (e.g., OX40, PD-L1) as the full-length antibody from which the portion or fragment is derived. It has been shown that the antigen-binding function of an antibody can be performed by fragments of a full-length antibody. Such antibody embodiments may also be bispecific, dual specific, or multi-specific formats; specifically binding to two or more different antigens (e.g., OX40 and a different antigen, such as PD-L1). Examples of binding fragments encompassed within the term “antigen-binding portion” of an antibody include (i) a Fab fragment, a monovalent fragment consisting of the VL, VH, CL, and CH1 domains; (ii) a F(ab′)₂ fragment, a bivalent fragment comprising two Fab fragments linked by a disulfide bridge at the hinge region; (iii) an Fd fragment consisting of the VH and CH1 domains; (iv) a Fv fragment consisting of the VL and VH domains of a single arm of an antibody, (v) a dAb fragment (Ward et al., Nature, 341:544-546 (1989); PCT Publication No. WO 90/05144), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, for example, Bird et al., Science, 242:423-426 (1988); and Huston et al., Proc. Natl. Acad. Sci. USA, 85:5879-5883 (1988)). Such single chain antibodies are also intended to be encompassed within the term “antigen-binding portion” of an antibody and equivalent terms given above. Other forms of single chain antibodies, such as diabodies are also encompassed. Diabodies are bivalent, bispecific antibodies in which VH and VL domains are expressed on a single polypeptide chain but using a linker that is too short to allow for pairing between the two domains on the same chain, thereby forcing the domains to pair with complementary domains of another chain and creating two antigen binding sites (see, for example, Holliger et al., Proc. Natl. Acad. Sci. USA, 90:6444-6448 (1993)). Such antibody binding portions are known in the art (Kontermann and Dübel eds., Antibody Engineering (Springer-Verlag, New York, 2001), p. 790 (ISBN 3-540-41354-5)). In addition, single chain antibodies also include “linear antibodies” comprising a pair of tandem Fv segments (VH-CH1-VH-CH1) which, together with complementary light chain polypeptides, form a pair of antigen binding regions (Zapata et al., Protein Eng., 8(10): 1057-1062 (1995); and U.S. Pat. No. 5,641,870)).

An immunoglobulin constant (C) domain refers to a heavy (CH) or light (CL) chain constant domain. Murine and human IgG heavy chain and light chain constant domain amino acid sequences are known in the art.

The term “monoclonal antibody” or “mAb” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic determinant (epitope). Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method.

The term “human sequence”, in relation to the light chain constant domain CL, heavy chain constant domain CH, and Fc region of the antibody or the binding protein according to the present application, means the sequence is of, or from, human immunoglobulin sequence. The human sequence of the present disclosure may be native human sequence, or a variant thereof including one or more (for example, up to 20, 15, 10) amino acid residue changes.

The term “chimeric antibody” refers to antibodies that comprise heavy and light chain variable region sequences from one species and constant region sequences from another species, such as antibodies having murine heavy and light chain variable regions linked to human constant regions.

The term “CDR-grafted antibody” refers to antibodies that comprise heavy and light chain variable region sequences from one species but in which the sequences of one or more of the CDR regions of VH and/or VL are replaced with CDR sequences of another species, such as antibodies having human heavy and light chain variable regions in which one or more of the human CDRs has been replaced with murine CDR sequences.

The term “humanized antibody” refers to antibodies that comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which CDR sequences from a non-human species (e.g., mouse) are introduced into human VH and VL framework sequences. A humanized antibody is an antibody or a variant, derivative, analog or fragment thereof which immunospecifically binds to an antigen of interest and which comprises framework regions and constant regions having substantially the amino acid sequence of a human antibody but complementarity determining regions (CDRs) having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

A humanized antibody may be selected from any class of immunoglobulins, including IgM, IgG, IgD, IgA and IgE, and any isotype, including without limitation IgG1, IgG2, IgG3, and IgG4. The humanized antibody may comprise sequences from more than one class or isotype, and particular constant domains may be selected to optimize desired effector functions using techniques well known in the art.

The framework and CDR regions of a humanized antibody need not correspond precisely to the parental sequences, e.g., the donor antibody CDR or the acceptor framework may be mutagenized by substitution, insertion and/or deletion of at least one amino acid residue so that the CDR or framework residue at that site does not correspond to either the donor antibody or the consensus framework. In an exemplary embodiment, such mutations, however, will not be extensive. Usually, at least 80%, at least 85%, at least 90%, or at least 95% of the humanized antibody residues will correspond to those of the parental FR and CDR sequences. Back mutation at a particular framework position to restore the same amino acid that appears at that position in the donor antibody is often utilized to preserve a particular loop structure or to correctly orient the CDR sequences for contact with target antigen.

The term “CDR” refers to the complementarity determining regions within antibody variable domain sequences. There are three CDRs in each of the variable regions of the heavy chain and the light chain, which are designated CDR-H1, CDR-H2, CDR-H3, CDR-L1, CDR-L2, and CDR-L3. The term “CDR set” as used herein refers to a group of three CDRs that occur in a single variable region capable of binding the antigen. The exact boundaries of these CDRs have been defined differently following different systems. The system described by Kabat (Kabat et al., Sequences of Proteins of Immunological Interest (National Institutes of Health, Bethesda, Maryland (1991)) not only provides an unambiguous residue numbering system applicable to any variable region of an antibody, but also provides precise residue boundaries defining the three CDRs.

The growth and analysis of extensive public databases of amino acid sequences of variable heavy and light regions over the past twenty years have led to the understanding of the typical boundaries between framework regions (FRs) and CDR sequences within variable region sequences and have enabled persons skilled in the art to accurately determine the CDRs according to Kabat numbering, Chothia numbering, or other systems. See, e.g., Martin, “Protein Sequence and Structure Analysis of Antibody Variable Domains,” In Kontermann and Dübel, eds., Antibody Engineering (Springer-Verlag, Berlin, 2001), chapter 31, pages 432-433. The term “multivalent binding protein” denotes a binding protein comprising two or more antigen binding sites. A multivalent binding protein is, in certain cases, engineered to have three or more antigen binding sites, and is generally not a naturally occurring antibody.

The term “bispecific binding protein” (which can be used interchangeably with the term “bispecific antibody”, unless stated otherwise) refers to a binding protein capable of binding two targets of different specificity. FIT-Ig binding proteins of the present disclosure comprise four antigen binding sites and are typically tetravalent binding proteins. A FIT-Ig according to this disclosure binds both OX40 and PD-L1 and is bispecific.

A FIT-Ig binding protein comprising two long (heavy) V-C-V-C-Fc chain polypeptides and four short (light) V-C chain polypeptides forms a hexamer exhibiting four Fab antigen binding sites (VH-CH1 paired with VL-CL, sometimes notated VH-CH1::VL-CL). Each half of a FIT-Ig comprises a heavy chain polypeptide and two light chain polypeptides, and complementary immunoglobulin pairing of the VH-CH1 and VL-CL elements of the three chains results in two Fab-structured antigen binding sites, arranged in tandem. In the present disclosure, it is preferred that the immunoglobulin domains comprising the Fab elements are directly fused in the heavy chain polypeptide, without the use of interdomain linkers. That is, the N-terminal V-C element of the long (heavy) polypeptide chains is directly fused at its C-terminus to the N-terminus of another V-C element, which in turn is linked to a C-terminal Fc region. In bispecific FIT-Ig binding proteins, the tandem Fab elements may be reactive with different antigens. Each Fab antigen binding site comprises a heavy chain variable domain and a light chain variable domain with a total of six CDRs per antigen binding site.

A description of the design, expression, and characterization of FIT-Ig molecules is provided in PCT Publication WO 2015/103072, which is incorporated herein in its entirety. An example of such FIT-Ig molecules comprises a heavy chain and two different light chains. The heavy chain comprises the structural formula VL_{A}-CL-VH_{B}-CH1-Fc where CL is directly fused to VH_{B} (namely “Format LH”) or VH_{B}-CH1-VL_{A}-CL-Fc where CH1 is fused directly to VL_{A} (namely “Format HL”), and the two light polypeptide chains of the FIT-Ig correspondingly have the formulas VH_{A}-CH1 and VL_{B}-CL respectively; alternatively, the heavy chain comprises the structural formula VL_{B}-CL-VH_{A}-CH1-Fc where CL is directly fused to VH_{A} (for “Format LH”) or VH_{A}-CH1-VL_{B}-CL-Fc where CH1 is fused directly to VL_{B} (for “Format HL”), and the two light polypeptide chains of the FIT-Ig correspondingly have the formulas VL_{A}-CL and VH_{B}-CH1 respectively; wherein VL_{A} is a variable light domain from a parental antibody that binds antigen A, VL_{B} is a variable light domain from a parental antibody that binds antigen B, VH_{A} is a variable heavy domain from a parental antibody that binds antigen A, VH_{B} is a variable heavy domain from a parental antibody that binds antigen B, CL is a light chain constant domain, CH1 is a heavy chain constant domain, and Fc is an immunoglobulin Fc region (e.g., the C-terminal hinge-CH2-CH3 portion of a heavy chain of an IgG1 antibody). In bispecific FIT-Ig embodiments, antigen A and antigen B are different antigens, or different epitopes of the same antigen. In the present disclosure, one of A and B is OX40 and the other is PD-L1, for example, A is OX40 and B is PD-L1.

The term “kₒₙ” (also “Kon”, “kon”), as used herein, is intended to refer to the on-rate constant for association of a binding protein (e.g., an antibody) to an antigen to form an association complex, e.g., antibody/antigen complex, as is known in the art. The “kₒₙ” also is known by the terms “association rate constant”, or “ka”, as used interchangeably herein. This value indicates the binding rate of an antibody to its target antigen or the rate of complex formation between an antibody and antigen as is shown by the equation below:

Antibody (“Ab”)+Antigen (“Ag”)→Ab-Ag.

The term “k_{off}” (also “Koff”, “koff”), as used herein, is intended to refer to the off-rate constant for dissociation, or “dissociation rate constant”, of a binding protein (e.g., an antibody) from an association complex (e.g., an antibody/antigen complex) as is known in the art. This value indicates the dissociation rate of an antibody from its target antigen or separation of Ab-Ag complex over time into free antibody and antigen as shown by the equation below:

Ab+Ag←Ab-Ag.

The term “K_{D}” (also “K_{d}”), as used herein, is intended to refer to the “equilibrium dissociation constant”, and refers to the value obtained in a titration measurement at equilibrium, or by dividing the dissociation rate constant (k_{off}) by the association rate constant (kₒₙ). The association rate constant (kₒₙ), the dissociation rate constant (k_{off}), and the equilibrium dissociation constant (K_{D}) are used to represent the binding affinity of an antibody to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used (e.g., instrument available from BIAcore International AB, a GE Healthcare company, Uppsala, Sweden). Biolayer interferometry (BLI) using, e.g., the Octet® RED96 system (Pall FortéBio LLC), is another affinity assay technique. Additionally, a KinExA® (Kinetic Exclusion Assay) assay, available from Sapidyne Instruments (Boise, Idaho) can also be used.

The term “isolated nucleic acid” means a polynucleotide (e.g., of genomic, cDNA, or synthetic origin, or some combination thereof) that, by human intervention, is not associated with all or a portion of the polynucleotides with which it is found in nature; is operably linked to a polynucleotide that it is not linked to in nature; or does not occur in nature as part of a larger sequence.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the present disclosure is intended to include such other forms of expression vectors, such as viral vectors (e.g., replication defective retroviruses, adenoviruses and adeno-associated viruses), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequence. “Operably linked” sequences include both expression control sequences that are contiguous with the gene of interest and expression control sequences that act in trans or at a distance to control the gene of interest. The term “expression control sequence” as used herein refers to polynucleotide sequences that are necessary to affect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing, and can also include additional components whose presence is advantageous, for example, leader sequences and fusion partner sequences.

“Transformation”, as referred to herein, refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, transfection, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The term “recombinant host cell” (or simply “host cell”), is intended to refer to a cell into which exogenous DNA has been introduced. In an embodiment, the host cell comprises two or more (e.g., multiple) nucleic acids encoding antibodies, such as the host cells described in U.S. Pat. No. 7,262,028, for example. Such terms are intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells selected from any of the Kingdoms of life. In another embodiment, eukaryotic cells include protist, fungal, plant and animal cells. In another embodiment, host cells include but are not limited to the prokaryotic cell line Escherichia coli; mammalian cell lines CHO, HEK 293, Jurkat, COS, NS0, SP2 and PER.C6; the insect cell line Sf9; and the fungal cell Saccharomyces cerevisiae.

As used herein, the term “effective amount” refers to the amount of a therapy that is sufficient to reduce or ameliorate the severity and/or duration of a disorder or one or more symptoms thereof; prevent the advancement of a disorder; cause regression of a disorder; prevent the recurrence, development, or progression of one or more symptoms associated with a disorder; detect a disorder; or enhance or improve the prophylactic or therapeutic effect(s) of another therapy (e.g., prophylactic or therapeutic agent).

As used herein, “activation of T cells” or “T cells activation” refers to a core process of the cell-mediated immunity, in which a particular foreign antigen induces the cognate naïve T cells to respond thereto. T cells activation, which is reflected in the proliferation and/or differentiation of T cells and the production of large amounts of effector T cells (e.g., such as cytotoxic T lymphocytes), may results in, e.g., the reduction or elimination of the foreign antigen. The process is complex and regulated by many factors, for example, the immunosuppressive tumoral micro-environment. Signs of T cell activation, by which the process could be measured, include but not limited to: significantly increased secretion of IL-2 or IFN-γ from T cells, and/or increased antigen response (e.g., tumor clearance). Methods of measuring are known to the skilled in the art.

Antibodies, antigen binding fragments thereof, and binding proteins according to the present disclosure may be purified (for an intended use) by using one or more of a variety of methods and materials available in the art for purifying antibodies and binding proteins. Such methods and materials include, but are not limited to, affinity chromatography (e.g., using resins, particles, or membranes conjugated to Protein A, Protein G, Protein L, or a specific ligand of the antibody, antigen binding fragment thereof, or binding protein), ion exchange chromatography (for example, using ion exchange particles or membranes), hydrophobic interaction chromatography (“HIC”; for example, using hydrophobic particles or membranes), ultrafiltration, nanofiltration, diafiltration, size exclusion chromatography (“SEC”), low pH treatment (to inactivate contaminating viruses), and combinations thereof, to obtain an acceptable purity for an intended use. A non-limiting example of a low pH treatment to inactivate contaminating viruses comprises reducing the pH of a solution or suspension comprising an antibody, antigen binding fragment thereof, or binding protein of the present disclosure to pH 3.5 with 0.5 M phosphoric acid, at 18° C.-25° C., for 60 to 70 minutes. Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual, 2nd ed. (Cold Spring Harbor Laboratory Press, Cold Spring Harbor, N.Y., 1989).

### Anti-OX40 and Anti-PD-L1 Monospecific Antibodies

Anti-OX40 and anti-PD-L1 antibodies of the present disclosure may be produced by any number of techniques known in the art. See, e.g., WO2021/1034434, the contents of which are hereby incorporated by reference. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies of the present disclosure in either prokaryotic or eukaryotic host cells, expression of antibodies in eukaryotic cells, for instance, in mammalian host cells, is particularly contemplated, because such eukaryotic cells (e.g., mammalian cells) are more likely than prokaryotic cells to assemble and secrete a properly folded and immunologically active antibody.

In some embodiments, the mammalian host cells for expressing the recombinant antibodies of the present disclosure are Chinese Hamster Ovary (CHO) cells (including dhfr⁻ CHO cells, described in Urlaub and Chasin, Proc. Natl. Acad. Sci. USA, 77:4216-4220 (1980), used with a DHFR selectable marker, e.g., as described in Kaufman and Sharp, J. Mol. Biol., 159:601-621 (1982)), NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells, or further secretion of the antibody into the culture medium in which the host cells are grown. Antibodies can be recovered from the culture medium using standard protein purification methods.

Host cells can also be used to produce antigen binding fragments, such as Fab fragments or scFv molecules. It will be understood that variations on the above procedure are within the scope of the present disclosure. For example, it may be desirable to transfect a host cell with DNA encoding antigen binding fragments of either the light chain and/or the heavy chain of an antibody of this disclosure. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the present disclosure. In addition, bifunctional antibodies may be produced by crosslinking an antibody of the present disclosure to a second antibody or another functional moiety by standard chemical crosslinking methods. In an exemplary system for recombinant expression of an antibody, or antigen-binding portion thereof, of the present disclosure, a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows selection of CHO cells that have been transfected with the vector using methotrexate selection/amplification. The selected transfected host cells are cultured to allow expression of the antibody heavy and light chains, and intact antibody is recovered from the culture medium. Standard molecular biology techniques are used to prepare the recombinant expression vector, transfect the host cells, select for transfectants, culture the host cells, and recover the antibody from the culture medium. The present disclosure also provides a method of making a recombinant anti-OX40 or anti-PD-L1 antibody by culturing a transfected host cell of the present disclosure in a suitable culture medium until a recombinant antibody of the present disclosure is produced. Optionally, the method further comprises isolating the recombinant antibody from the culture medium.

### Anti-OX40 Antibodies

In some embodiments, the present disclosure provides antibodies that bind to OX40 at a membrane proximal CRD of the OX40 Ig-like domain. The antibodies disclosed herein, in some embodiments, have high cell binding potency and/or are characterized by low internalization rate, e.g., as measured in a cell-based assay.

In some embodiments, the present disclosure discloses an isolated anti-OX40 antibody or antigen-binding fragment thereof that specifically binds to OX40. In a further embodiment, the anti-OX40 antibody or antigen-binding fragment thereof comprises a set of six CDRs, CDR-H1, CDR-H2, CDR-H3, CDR-L1, CDR-L2, and CDR-L3, wherein:
CDR-H1 comprises the sequence of SSWMN (SEQ ID NO:1);
CDR-H2 comprises the sequence of RIYPGDEITNYNGKFKD (SEQ ID NO: 2) or RIYPGDEITNYNAKFKD (SEQ ID NO: 4);
CDR-H3 comprises the sequence of DLLMPY (SEQ ID NO: 3);
CDR-L1 comprises the sequence of RSSKSLLYSNGITYLY (SEQ ID NO: 5) or RSSKSLLYSNAITYLY (SEQ ID NO: 8);
CDR-L2 comprises the sequence of QMSNLAP (SEQ ID NO: 6); and
CDR-L3 comprises the sequence of AQNLELPFT (SEQ ID NO: 7),
wherein the CDRs are defined according to Kabat numbering.

In some embodiments, the anti-OX40 antibody or antigen-binding fragment thereof comprises, at positions H31-H35, H50-H66, and H99-H104 according to Kabat numbering, the amino acid sequences of CDR-H1, CDR-H2, and CDR-H3 selected from the group of consisting of: (i) SEQ ID NOs: 1, 2, 3; or (ii) SEQ ID NOs: 1, 4, 3.

In one embodiment, the anti-OX40 antibody or antigen-binding fragment thereof comprises, at positions L24-39, L55-61 and L94-102 according to Kabat numbering, the amino acid sequences of SEQ ID NOs: 5, 6 and 7 or SEQ ID NOs: 8, 6 and 7 for CDR-L1, CDR-L2, and CDR-L3, respectively.

In certain embodiments, the anti-OX40 antibody or antigen-binding fragment thereof comprises a G62A mutation in the VH domain according to Kabat numbering. In certain embodiments, the anti-OX40 antibody or antigen-binding fragment thereof comprises a G34A mutation in the VL domain according to Kabat numbering. In some embodiments, the mutations reduce the propensity of asparagine deamidation in the anti-OX40 antibody or antigen-binding fragment thereof. In some embodiments, the anti-OX40 antibody or antigen-binding fragment thereof with the mutations has increased stability relative to the parental antibody without the mutations.

In some embodiments, the anti-OX40 antibody or antigen-binding fragment thereof comprises at least one, two, three, four, but not more than five residue modifications in the CDR sequences of SEQ ID NOs: 1-3 and 5-7. In some embodiments, the anti-OX40 antibody or antigen-binding fragment thereof comprises at least one, two, three, four, but not more than five residue modifications in the CDR sequences of SEQ ID NOs: 1, 4, 3 and 5-7. In some embodiments, the anti-OX40 antibody or antigen-binding fragment thereof comprises at least one, two, three, four, but not more than five residue modifications in the CDR sequences of SEQ ID NOs: 1-3 and 8, 6, 7. In some embodiments, the anti-OX40 antibody or antigen-binding fragment thereof comprises at least one, two, three, four, but not more than five residue modifications in the CDR sequences of SEQ ID NOs: 1, 4, 3 and 8, 6, 7. The amino acid modifications may be amino acid substitutions, deletions, and/or additions, for instance, conservative substitutions. In one embodiment, an anti-OX40 antibody or antigen-binding fragment thereof according to the present disclosure comprises CDR-H1, CDR-H2, CDR-H3, CDR-L1, CDR-L2, and CDR-L3 of a heavy chain variable domain VH and a light chain variable domain VL, selected from the group consisting of the following VH/VL sequence pairs: SEQ ID NOs: 11/19, 12/19, 13/19, 14/19, 11/20, 12/20, 13/20, 14/20, 10/17, 9/18, 10/18, 9/19, 11/17, 15/21, 15/18, 16/21 and 16/18. The CDRs can be determined by a person skilled in the art using the most widely CDR definition schemes, for example, Kabat, Chothia or IMGT definitions.

In one embodiment, an anti-OX40 antibody or antigen-binding fragment thereof according to the present disclosure comprises a heavy chain variable domain VH and a light chain variable domain VL, wherein:
the VH domain comprises the sequence of SEQ ID NO: 9 or 10, or a sequence having at least 80%, 85%, 90%, 91%, 92%, 93%, 94%, 95%, 96%, 97%, 98%, 99% or more identity therewith, and/or
the VL domain comprises the sequence of SEQ ID NO: 17 or 18, or a sequence having at least 80%, 85%, 90%, 91%, 92%, 93%, 94%, 95%, 96%, 97%, 98%, 99% or more identity therewith.

In another embodiment, an anti-OX40 antibody or antigen-binding fragment thereof according to the present disclosure comprises a heavy chain variable domain VH and a light chain variable domain VL, wherein:
the VH domain comprises the sequence selected from SEQ ID NOs: 11-16, or a sequence having at least 80%, 85%, 90%, 91%, 92%, 93%, 94%, 95%, 96%, 97%, 98%, 99% or more identity therewith, and/or
the VL domain comprises the sequence selected from SEQ ID NOs: 19-21, or a sequence having at least 80%, 85%, 90%, 91%, 92%, 93%, 94%, 95%, 96%, 97%, 98%, 99% or more identity therewith.

In some embodiments, an anti-OX40 antibody comprises a VH sequence having at least 80%, 85%, 90%, 91%, 92%, 93%, 94%, 95%, 96%, 97%, 98%, or 99% identity contains substitutions (e.g., conservative substitutions), insertions, or deletions relative to the reference sequence, while retains the ability to bind to the OX40 with the same or improved binding properties, such as the off-rate and/or the on-rate. In some embodiments, a total of 1 to 11 amino acids have been substituted, inserted and/or deleted in SEQ ID NO: 9, 10, or any one of SEQ ID NOs: 11-16. In certain embodiments, substitutions, insertions, or deletions occur in regions outside the CDRs (i.e., in the FRs). Optionally, the anti-OX40 antibody comprises the VH sequence of SEQ ID NO: 9, 10, or any one of SEQ ID NOs: 11-16, including post-translational modifications of that sequence. In a particular embodiment, the VH comprises one, two or three CDRs selected from: (a) a CDR-H1 comprising the amino acid sequence of SEQ ID NO: 1, (b) a CDR-H2 comprising the amino acid sequence of SEQ ID NO: 2 or 4, and (c) a CDR-H3 comprising the amino acid sequence of SEQ ID NO: 3. In some embodiments, the VH sequence is a humanized VH sequence.

In some embodiments, an anti-OX40 antibody comprises a VL sequence having at least 80%, 85%, 90%, 91%, 92%, 93%, 94%, 95%, 96%, 97%, 98%, or 99% identity contains substitutions (e.g., conservative substitutions), insertions, or deletions relative to the reference sequence, while retains the ability to bind to the OX40 with the same or improved binding properties, such as the off-rate and/or the on-rate. In some embodiments, a total of 1 to 5 amino acids have been substituted, inserted and/or deleted in SEQ ID NO: 17, 18, or any one of SEQ ID NOs: 19-21. In certain embodiments, substitutions, insertions, or deletions occur in regions outside the CDRs (i.e., in the FRs). Optionally, the anti-OX40 antibody comprises the VL sequence of SEQ ID NO: 17, 18, or any one of SEQ ID NOs: 19-21, including post-translational modifications of that sequence. In a particular embodiment, the VL sequence comprises one, two or three CDRs selected from: (a) a CDR-L1 comprising the amino acid sequence of SEQ ID NO: 5 or SEQ ID NO: 8, (b) a CDR-L2 comprising the amino acid sequence of SEQ ID NO: 6, and (c) a CDR-L3 comprising the amino acid sequence of SEQ ID NO: 7. In some embodiments, the VL sequence is a humanized VL sequence.

In one embodiment, an anti-OX40 antibody or antigen-binding fragment thereof according to the present disclosure comprises a heavy chain variable domain VH comprising or consisting of SEQ ID NO: 16, and a light chain variable domain VL comprising or consisting of SEQ ID NO: 21.

In one embodiment, the isolated anti-OX40 antibody or antigen-binding fragment according to the present disclosure is a chimeric antibody or a humanized antibody. In some embodiments, the anti-OX40 antibody or antigen-binding fragment is a humanized antibody.

In some embodiments, the humanized isolated anti-OX40 antibody or antigen-binding fragment according to the present disclosure comprises one or more back mutations at positions in framework regions to improve the binding property. In some embodiments, the VH domain of the humanized anti-OX40 antibody or antigen-binding fragment according to the present disclosure comprises back mutations from human to residues: a Glu at position 1 (1E), and optionally one or more of a Gln at position 5 (5Q), a His at position 27 (27H), an Ala at position 28 (28A), a Lys at position 38 (38K), an Arg at position 40 (40R), a Lys at position 43 (43K), an Ile at position 48 (48I), a Lys at position 67 (67K), an Ala at position 68 (68A), and a Leu at position 70 (70L) according to Kabat numbering. In one embodiment, the VL domain of the humanized anti-OX40 antibody or antigen-binding fragment according to the present disclosure optionally comprises back mutations from human to residue: a Ser at position 69 (69S) according to Kabat numbering.

In one embodiment, the isolated anti-OX40 antibody or antigen-binding fragment according to the present disclosure is a humanized antibody, comprising back-mutated amino acid residues in the VH domain selected from the group consisting of: (i) 1E, (ii) 1E and 27H, (iii) 1E, 27H, 48I, and 70L, (iv) 1E, 27H, 38K, 43K, 48I, 67K, and 70L, (v) 1E, 40R, and 43K, (vi) 1E, 5Q, 27H, 28A, 38K, 40R, 43K, 48I, 67K, 68A, and 70L, all according to Kabat numbering; and/or back-mutated amino acid residue of 69S in the VL domain according to Kabat numbering.

In one embodiment, the isolated anti-OX40 antibody or antigen-binding fragment according to the present disclosure is a humanized antibody, comprising amino acid residues 1E, 5Q, 27H, 28A, 38K, 40R, 43K, 48I, 67K, 68A, and 70L in the VH domain, and amino acid residue 69S in the VL domain, according to Kabat numbering. In a further embodiment, the isolated anti-OX40 antibody or antigen-binding fragment according to the present disclosure further comprises G62A mutation in the VH domain according to Kabat numbering and G34A mutation in the VL domain according to Kabat numbering.

In some embodiments, the isolated anti-OX40 antibody or antigen-binding fragment according to the present disclosure comprises a combination of VH and VL sequences selected from the group consisting of:

| | VH sequence, | VL sequence, |
|---|---|---|
| | which comprises | which comprises |
| combination | or consists of | or consists of |
| 1 | SEQ ID NO: 11 | SEQ ID NO: 19 |
| 2 | SEQ ID NO: 12 | SEQ ID NO: 19 |
| 3 | SEQ ID NO: 13 | SEQ ID NO: 19 |
| 4 | SEQ ID NO: 14 | SEQ ID NO: 19 |
| 5 | SEQ ID NO: 11 | SEQ ID NO: 20 |
| 6 | SEQ ID NO: 12 | SEQ ID NO: 20 |
| 7 | SEQ ID NO: 13 | SEQ ID NO: 20 |
| 8 | SEQ ID NO: 14 | SEQ ID NO: 20 |
| 9 | SEQ ID NO: 10 | SEQ ID NO: 17 |
| 10 | SEQ ID NO: 9 | SEQ ID NO: 18 |
| 11 | SEQ ID NO: 10 | SEQ ID NO: 18 |
| 12 | SEQ ID NO: 9 | SEQ ID NO: 19 |
| 13 | SEQ ID NO: 11 | SEQ ID NO: 17 |
| 14 | SEQ ID NO: 15 | SEQ ID NO: 21 |
| 15 | SEQ ID NO: 15 | SEQ ID NO: 18 |
| 16 | SEQ ID NO: 16 | SEQ ID NO: 21 |
| 17 | SEQ ID NO: 16 | SEQ ID NO: 18 |

In some embodiments, the antibody comprises a VH domain comprising or consisting of the sequence of SEQ ID NO: 16, and a VL domain comprising or consisting of the sequence of SEQ ID NO: 21.

In some embodiments of an anti-OX40 antibody or antigen-binding fragment according to the present disclosure, the antibody or antigen-binding fragment comprises an Fc region, which may be a native or a variant Fc region. In particular embodiments, the Fc region is a human Fc region from IgG1, IgG2, IgG3, IgG4, IgA, IgM, IgE, or IgD. Depending on the utility of the antibody, it may be desirable to use a variant Fc region to change (for example, reduce or eliminate) at least one effector function, for example, ADCC and/or CDC. In some embodiments, the present disclosure provides an anti-OX40 antibody or antigen-binding fragment comprising an Fc region with one or more mutation to change at least one effector function, for example, L234A and L235A.

In some embodiments, antigen-binding fragments of an anti-OX40 antibody according to the present disclosure may be for example, Fv, Fab, Fab′, Fab′-SH, F(ab′)2; diabodies; linear antibodies; or single-chain antibody molecules (e.g. scFv).

In one embodiment, an anti-OX40 antibody described herein or an antigen-binding fragment thereof binds to the OX40 extracellular domain or a portion thereof. In some embodiments, the OX40 extracellular domain comprises the amino acid squence L29-A214 of the human OX40 protein under UniProt Identifier P43489, or the amino acid sequence of SEQ ID NO: 44, or a sequence having at least 80%, 85%, 90%, 91%, 92%, 93%, 94%, 95%, 96%, 97%, 98%, 99% or more identity therewith:

(SEQ ID NO: 44)
MCVGARRLGRGPCAALLLLGLGLSTVTGLHCVGDTYPSNDRCCHECRPGN
GMVSRCSRSQNTVCRPCGPGFYNDVVSSKPCKPCTWCNLRSGSERKQLCT
ATQDTVCRCRAGTQPLDSYKPGVDCAPCPPGHFSPGDNQACKPWTNCTLA
GKHTLQPASNSSDAICEDRDPPATQPQETQGPPARPITVQPTEAWPRTSQ
GPSTRPVEVPGGRAVAAILGLGLVLGLLGPLAILLALYLLRRDQRLPPDA
HKPPGGGSFRTPIQEEQADAHSTLAKI

In one embodiment, an anti-OX40 antibody described herein or an antigen-binding fragment thereof binds to OX40 at the CRD3 region of the OX40 extracellular domain.

In an embodiment, an anti-OX40 antibody described herein or an antigen-binding fragment thereof has an on-rate constant (kₒₙ) to human OX40 of at least 1×10⁴ M⁻¹ s⁻¹, at least 3×10⁴ M⁻¹ s⁻¹, at least 5×10⁴ M⁻¹ s⁻¹, at least 7×10⁴ M⁻¹ s⁻¹, at least 9×10⁴ M⁻¹ s⁻¹, at least 1×10⁵ M 1 s⁻¹, as measured by biolayer interferometry or surface plasmon resonance.

In another embodiment, an anti-OX40 antibody described herein or an antigen-binding fragment thereof has an off-rate constant (k_{off}) to human OX40 of less than 5×10⁻³ s⁻¹, less than 3×10⁻³ s⁻¹, less than 2×10⁻³ s⁻¹, less than 1×10⁻³ s⁻¹, less than 9×10⁻⁴ s⁻¹, less than 6×10⁻⁴ s⁻¹, less than 3×10⁻⁴ s⁻¹, less than 2.5×10⁻⁴ s⁻¹, less than 2×10⁻⁴ s⁻¹, less than 1×10⁻⁴ s⁻¹, less than 8×10⁻⁵ s⁻¹, less than 5×10⁻⁵ s⁻¹, as measured by surface plasmon resonance or biolayer interferometry. In a further embodiment, an anti-OX40 antibody described herein or an antigen-binding fragment thereof is a humanized antibody, and has a k_{off} for human OX40 that is about 50-500%, for example about 80-150% of the k_{off} value for human OX40 of an antibody with a VH and VL sequence pair of SEQ ID NOs: 9/10 and 17/18 in the same antibody format. In general, a long off-rate correlates with a slow dissociation of the formed complex whereas a short off-rate correlates with a quick dissociation. In one embodiment, the anti-OX40 antibody described herein, or antigen-binding fragment thereof, has an affinity to the target OX40 higher than that of 1A7.gr.1 as described in WO2015153513, as indicated by a lower off-rate.

In one embodiment, an anti-OX40 antibody described herein or an antigen-binding fragment thereof has a dissociation constant (K_{D}) to OX40 in the nanomolar to picomolar (10⁻⁸ to 10⁻¹⁰) range, for example, less than 8×10⁻⁸ M, less than 5×10⁻⁸ M, less than 3×10⁻⁸ M, less than 1×10⁻⁸ M, less than 8×10⁻⁹M, less than 5×10⁻⁹M, less than 3×10⁻⁹ M, less than 2×10⁻⁹ M, less than 1×10⁻⁹M, less than 8×10⁻¹⁰M, less than 6×10⁻¹⁰M, less than 4×10⁻¹⁰M, less than 2×10⁻¹⁰ M, or less than 1×10⁻¹⁰ M.

In one embodiment, an anti-OX40 antibody described herein or an antigen-binding fragment thereof specifically binds to OX40 displayed on OX40⁺ target cells, such as CHO cell lines or T cell lines (e.g., primary T cells, and Jurkat) expressing OX40. As measured by flow cytometry in a cell-based assay, the anti-OX40 antibody displays strong binding potency to OX40⁺ cells, wherein said cell binding potency is reflected by an EC50 of about 5 nM or lower, 4 nM or lower, 3 nM or lower, 2 nM or lower, or 1 nM or lower. In a further embodiment, the EC50 is 0.5 nM or lower. In some embodiments, the anti-OX40 antibody or antigen-binding fragment described herein displays a similar or higher binding potency to OX40 displayed on the target cell, as compared to an antibody with a VH and VL sequence pair of SEQ ID NOs: 9/10 and 17/18. In one embodiment, the binding potency of an antibody to OX40-expressing cells is measured in a cell-based assay as described in Example 1.2. In some embodiments, the binding of the anti-OX40 antibody described herein or an antigen-binding fragment thereof to OX40 with a binding potency above-mentioned is sufficient to induce a cellular effect in vivo or in vitro. In a further embodiment, the effect is activation and/or proliferation of T cells.

In one embodiment, the antibody can bind OX40 as its ligand OX40L does on the cell surface of OX40-expressing cells. In another embodiment, the antibody can be used for enhancing OX40/OX40L signaling. In a further embodiment, the antibody can be used for inducing and/or enhancing T cell activation and proliferation associated with OX40/OX40L pathway.

### Anti-PD-L1 Antibodies

The present disclosure also provides antibodies capable of binding human PD-L1.

In some embodiments, an anti-PD-L1 antibody according to the present disclosure comprises: a set of six CDRs, CDR-H1, CDR-H2, CDR-H3, CDR-L1, CDR-L2, and CDR-L3, wherein:
CDR-H1 comprises the sequence of TYGIN (SEQ ID NO:22);
CDR-H2 comprises the sequence of YIYIGNAYTEYNEKFKG (SEQ ID NO: 23) or YIYIGNGYTEYNEKFKG (SEQ ID NO: 25);
CDR-H3 comprises the sequence of DLMVIAPKTMDY (SEQ ID NO: 24);
CDR-L1 comprises the sequence of KASQDVGTAVA (SEQ ID NO: 26);
CDR-L2 comprises the sequence of WASTRHT (SEQ ID NO: 27); and
CDR-L3 comprises the sequence of QQYSSYPYT (SEQ ID NO: 28),
wherein the CDRs are defined according to Kabat numbering.

In some embodiments, the anti-PD-L1 antibody or antigen-binding fragment thereof according to the present application comprises:
a VH domain comprising the sequence of SEQ ID NO: 29, 30, or 31 or a sequence having at least 80%-90%, or 95%-99% identity therewith, and/or
a VL domain comprising the sequence of SEQ ID NO: 32, 33, or 34, or a sequence having at least 80%-90%, or 95%-99% identity therewith.

In some embodiments, the anti-PD-L1 antibody or antigen-binding fragment thereof comprises a VH domain comprising the sequence of SEQ ID NO: 31 and a VL domain comprising the sequence of SEQ ID NO: 34.

In some embodiments, an anti-OX40 antibody according to the present disclosure or an anti-PD-L1 antibody according to the present disclosure may be used to make derivative binding proteins recognizing the same target antigen by techniques well established in the field. Such a derivative may be, e.g., a single-chain antibody (scFv), a Fab fragment (Fab), a Fab′ fragment, an F(ab′)₂, an Fv, and a disulfide linked Fv. Such a derivative may be, e.g., a fusion protein or conjugate comprising the anti-OX40 antibody according to the present disclosure or an anti-PD-L1 antibody according to the present disclosure. The fusion protein may be a multi-specific antibody or a CAR molecule. The conjugate may be an antibody-drug conjugate (ADC), or an antibody conjugated with a detection agent such as a radioisotope.

In one embodiment, an anti-PD-L1 antibody described herein or an antigen-binding fragment thereof has a dissociation constant (K_{D}) to PD-L1, such as human PD-L1, at sub-nanomolar level, for example, less than 1×10⁻⁹M, less than 8×10⁻¹⁰M, less than 6×10⁻¹⁰M, less than 4×10⁻¹⁰ M, less than 3×10⁻¹⁰ M. In one embodiment, an anti-PD-L1 antibody described herein or an antigen-binding fragment thereof specifically binds to PD-L1 displayed on PD-L1+ target cells. As measured by flow cytometry, Bio-Layer Interferometry, and/or surface plasmon resonance, the anti-PD-L1 antibody displays strong binding potency to PD-L1⁺ cells. Said cell binding potency to human PD-L1 is similar with that to Cynomolgus PD-L1, for example, <5-fold difference or <3-fold difference, according to EC50 by FACS binding and/or K_{D} by BLI or BIAcore.

### OX40×PD-L1 Bispecific Binding Proteins

In another aspect, the present disclosure provides OX40/PD-L1 bispecific binding proteins, especially Fabs-in-Tandem immunoglobulins (FIT-Ig), that are capable of binding to both OX40 and PD-L1. Each variable domain (VH or VL) in a FIT-Ig may be obtained from one or more “parental” monoclonal antibodies that bind one of the target antigens, i.e., OX40 or PD-L1. FIT-Ig binding proteins may be produced using variable domain sequences of anti-OX40 and anti-PD-L1 monoclonal antibodies as disclosed herein, for instance, humanized anti-OX40 and humanized anti-PD-L1 parental antibodies.

One aspect of the present disclosure pertains to selecting parental antibodies with at least one or more properties desired in the FIT-Ig molecule. In an embodiment, the antibody properties are selected from the group consisting of antigen specificity, affinity to antigen, dissociation rate, cell binding potency, biological function, epitope recognition, stability, solubility, production efficiency, immunogenicity, pharmacokinetics, bioavailability, tissue cross reactivity, orthologous antigen binding, and so on.

In some embodiments, bispecific FIT-Ig proteins according to the present disclosure are configured without any interdomain peptide linker. Whereas in multivalent engineered immunoglobulin formats having tandem binding sites, it was commonly understood in the field that the adjacent binding sites would interfere with each other unless a flexible linker was used to separate the binding sites spatially. It has been discovered for the OX40/PD-L1 FIT-Ig of the present disclosure, however, that the arrangement of the immunoglobulin domains according to the chain formulas disclosed herein results in polypeptide chains that are well-expressed in transfected mammalian cells, assembled appropriately, and are secreted as intact bispecific, multivalent immunoglobulin-like binding proteins that bind the target antigens OX40 and PD-L1. See, Examples, infra. Moreover, omission of synthetic linker sequences from the binding proteins can avoid the creation of antigenic sites recognizable by mammalian immune systems, and in this way the elimination of linkers decreases possible immunogenicity of the FIT-Igs and leads to a half-life in circulation that is like a natural antibody.

In some embodiments, an OX40×PD-L1 bispecific binding protein according to the present application comprises:
a) a first antigen-binding site that specifically binds OX40; and
b) a second antigen-binding site that specifically binds PD-L1.

In one embodiment, the bispecific binding proteins as described herein comprise a set of six CDRs, CDR-H1, CDR-H2, CDR-H3, CDR-L1, CDR-L2, and CDR-L3 derived from any anti-OX40 antibody or antigen-binding fragment thereof according to the present application and described herein to form the OX40 binding site of the bispecific binding protein. In some further embodiments, the bispecific binding proteins as described herein comprise a VH/VL pair derived from any anti-OX40 antibody or antigen-binding fragment thereof according to the present application and described herein to form the OX40 binding site of the bispecific binding protein.

In one embodiment, the bispecific binding proteins as described herein further comprise a set of six CDRs, CDR-H1, CDR-H2, CDR-H3, CDR-L1, CDR-L2, and CDR-L3 derived from any anti-PD-L1 antibody or antigen-binding fragment thereof according to the present application and described herein to form the PD-L1 binding site of the bispecific binding protein. In some further embodiments, the bispecific binding proteins as described herein comprise a VH/VL pair derived from any anti-PD-L1 antibody or antigen-binding fragment thereof according to the present application and described herein to form the PD-L1 binding site of the bispecific binding protein.

In one embodiment, the OX40 binding site and the PD-L1 binding site in a bispecific OX40/PD-L1 binding protein according to the present application are humanized, comprising humanized VH/VL sequences, respectively.

### Bispecific FIT-Ig Binding Proteins

In one embodiment, an OX40×PD-L1 bispecific binding protein according to the present application is a bispecific FIT-Ig binding protein capable of binding OX40 and PD-L1. A Fabs-in-Tandem immunoglobulin (FIT-Ig) binding protein is a dual-specific, tetravalent binding protein comprising six polypeptide chains, and having four functional Fab binding regions with two outer Fab binding regions and two inner Fab binding regions. The binding protein adopts the format (outer Fab−inner Fab−Fc)×2, and binds both antigen A and antigen B. In one aspect, the OX40×PD-L1 bispecific binding protein according to the present application is a bispecific FIT-Ig binding protein, wherein two Fab domains of the FIT-Ig protein confer first antigen-binding sites that specifically bind OX40; and the other two Fab domains of the FIT-Ig protein confer second antigen-binding sites that specifically bind PD-L1. In some embodiments, a FIT-Ig binding protein according to the present disclosure employs no linker between immunoglobulin domains.

In one embodiment, the binding protein comprises a first polypeptide comprising, from amino to carboxyl terminus, VL_{A}-CL-VH_{B}-CH1-Fc or VH_{B}-CH1-VL_{A}-CL-Fc, a second polypeptide comprising, from amino to carboxyl terminus, VH_{A}-CH1, and a third polypeptide comprising, from amino to carboxyl terminus, VL_{B}-CL, alternatively, the binding protein comprises a first polypeptide comprising, from amino to carboxyl terminus, VL_{B}-CL-VH_{A}-CH1-Fc or VH_{A}-CH1-VL_{B}-CL-Fc, a second polypeptide comprising, from amino to carboxyl terminus, VH_{B}-CH1, and a third polypeptide comprising, from amino to carboxyl terminus, VL_{A}-CL; wherein VL stands for a light chain variable domain, CL stands for a light chain constant domain, VH stands for a heavy chain variable domain, CH1 stands for the first constant domain of the heavy chain, A stands for OX40, and B stands for PD-L1. Each bispecific binding protein is a hexamer comprising two said first polypeptide, two said second polypeptide, and two said third polypeptide, exhibiting four Fab antigen binding sites, two for binding OX40 (VH_{A}-CH1 paired with VL_{A}-CL, noted VH_{A}-CH1::VL_{A}-CL) and two for binding PD-L1 (VH_{B}-CH1 paired with VL_{B}-CL, noted VH_{B}-CH1::VL_{B}-CL).

In some embodiments, the Fab binding to OX40 formed by VL-CL pairing with VH-CH1 in the FIT-Ig binding protein (for example, when A is OX40, formed by VL_{A}-CL and VH_{A}-CH1; or when B is OX40, formed by VL_{B}-CL and VH_{B}-CH1) comprises a set of six CDRs, namely CDR-H1, CDR-H2, CDR-H3, CDR-L1, CDR-L2, and CDR-L3, derived from any anti-OX40 antibody or antigen-binding fragment thereof according to the present application and described herein to form the OX40 binding site of the bispecific binding protein. In some further embodiments, the CDR-H1, CDR-H2, CDR-H3, CDR-L1, CDR-L2, and CDR-L3 comprise respectively the sequences of SEQ ID NOs: 1, 2, 3 and 5, 6, 7; the sequences of SEQ ID NOs: 1, 4, 3 and 5, 6, 7; the sequences of SEQ ID NOs: 1, 2, 3 and 8, 6, 7; or the sequences of SEQ ID NOs: 1, 4, 3 and 8, 6, 7.

In some embodiments, the Fab binding to OX40 in the FIT-Ig binding protein comprises a VH/VL pair derived from any anti-OX40 antibody or antigen-binding fragment thereof according to the present application and described herein. In some further embodiments, the VH/VL pair comprises the sequences selected from the group consisting of the following VH/VL sequence pairs: SEQ ID NOs: 11/19, 12/19, 13/19, 14/19, 11/20, 12/20, 13/20, 14/20, 10/17, 9/18, 10/18, 9/19, 11/17, 15/21, 15/18, 16/21 and 16/18, or sequences having at least 80%, 85%, 90%, 95% or 99% identity therewith. In some embodiments, the Fab binding to OX40 in the FIT-Ig binding protein comprises a VH sequence of SEQ ID NO: 16 and a VL sequence of SEQ ID NO: 21.

In some embodiments, the Fab binding to PD-L1 formed by VL-CL pairing with VH-CH1 in the FIT-Ig binding protein (for example, when A is PD-L1, formed by VL_{A}-CL and VH_{A}-CH1; or when B is PD-L1, formed by VL_{B}-CL and VH_{B}-CH1) comprises a set of six CDRs, namely CDR-H1, CDR-H2, CDR-H3, CDR-L1, CDR-L2, and CDR-L3, derived from any anti-PD-L1 antibody or antigen-binding fragment thereof according to the present application and described herein to form the PD-L1 binding site of the bispecific binding protein. In some embodiments, the Fab binding to PD-L1 formed by VL-CL pairing with VH-CH1 in the FIT-Ig binding protein comprises a set of six CDRs, wherein CDR-H1, CDR-H2, CDR-H3, CDR-L1, CDR-L2, and CDR-L3 comprise respectively the sequences of SEQ ID NOs: 22, 23, 24 and 26, 27, 28; or the sequences of SEQ ID NOs: 22, 25, 24 and 26, 27, 28. In some further embodiments, the Fab binding to PD-L1 comprises a VH/VL pair comprising the sequences of SEQ ID NOs: 31 and 34, or sequences having at least 80%, 85%, 90%, 95% or 99% identity therewith.

In the present disclosure, an OX40/PD-L1 FIT-Ig binding protein comprises first, second, and third polypeptide chains, wherein the first polypeptide chain comprises, from amino to carboxyl terminus, VL_{OX40}-CL-VH_{PD-L1}-CH1-Fc with CL directly fused to VH_{PD-L1} or VH_{PD-L1}-CH1-VL_{OX40}-CL-Fc with CH1 is directly fused to VL_{OX40}, wherein the second polypeptide chain comprises, from amino to carboxyl terminus, VH_{OX40}-CH1; and wherein the third polypeptide chain comprises, from amino to carboxyl terminus, VL_{PD-L1}-CL. In alternative embodiments, an OX40/PD-L1 FIT-Ig binding protein comprises first, second, and third polypeptide chains, wherein the first polypeptide chain comprises, from amino to carboxyl terminus, VH_{OX40}-CH1-VL_{PD-L1}-CL-Fc with CH1 directly fused to VL_{PD-L1} or VL_{PD-L1}-CL-VH_{OX40}-CH1-Fc with CL directly fused to VH_{OX40}, wherein the second polypeptide chain comprises, from amino to carboxyl terminus, VH_{PD-L1}-CH1; and wherein the third polypeptide chain comprises, from amino to carboxyl terminus, VL_{OX40}-CL. In some embodiments, VL_{OX40} is a light chain variable domain of an anti-OX40 antibody, CL is a light chain constant domain, VH_{OX40} is a heavy chain variable domain of an anti-OX40 antibody, CH1 is a heavy chain constant domain, VL_{PD-L1} is a light chain variable domain of an anti-PD-L1 antibody, VH_{PD-L1} is a heavy chain variable domain of an anti-PD-L1 antibody; and optionally, the domains VL_{PD-L1}-CL are the same as the light chain of an anti-PD-L1 parental antibody, the domains VH_{PD-L1}-CH1 are the same as the heavy chain variable and heavy chain constant domains of an anti-PD-L1 parental antibody, the domains VL_{OX40}-CL are the same as the light chain of an anti-OX40 parental antibody, and the domains VH_{OX40}-CH1 are the same as the heavy chain variable and heavy chain constant domains of an anti-OX40 parental antibody.

In one embodiment, the VH_{OX40}-CH1 comprises an amino acid sequence of at least 80%, 85%, 90%, 91%, 92%, 93%, 94%, 95%, 96%, 97%, 98%, 99% or 100% identity to SEQ ID NO: 38.

SEQ ID NO: 38:
EVQLQQSGAEVKKPGSSVKVSCKASGHAFSSSWMNWVKQRPGKGLEWIGR
IYPGDEITNYNAKFKDKATLTADKSTSTAYMELSSLRSEDTAVYYCARDL
LMPYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSC

In one embodiment, the VL_{OX40}-CL comprises an amino acid sequence of at least 80%, 85%, 90%, 91%, 92%, 93%, 94%, 95%, 96%, 97%, 98%, 99%, or 100% identical to SEQ ID NO: 37.

SEQ ID NO: 37:
DIVMTQTPLSLPVTPGEPASISCRSSKSLLYSNAITYLYWYLQKPGQSPQ
LLIYQMSNLAPGVPDRFSSSGSGTDFTLKISRVEAEDVGVYYCAQNLELP
FTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

In one embodiment, the VH_{PDL1}-CH1 comprises an amino acid sequence of at least 80%, 85%, 90%, 91%, 92%, 93%, 94%, 95%, 96%, 97%, 98%, 99% or 100% identity to SEQ ID NO: 36.

SEQ ID NO: 36:
EVQLVQSGSELKKPGASVKVSCKASGYTFTTYGINWVRQAPGQGLEWMGY
IYIGNAYTEYNEKFKGRFVFSLDTSVSTAYLQISSLKAEDTAVYYCARDL
MVIAPKTMDYWGQGTTVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSC

In one embodiment, the VL_{PDL1}-CL comprises an amino acid sequence of at least 80%, 85%, 90%, 91%, 92%, 93%, 94%, 95%, 96%, 97%, 98%, 99%, or 100% identity to SEQ ID NO: 39.

SEQ ID NO: 39:
DIQMTQSPSSVSASVGDRVTITCKASQDVGTAVAWYQQKPGKAPKLLIYW
ASTRHTGVPSRFSGSGSGTDFTLTISSLQPEDFATYYCQQYSSYPYTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In the foregoing formulas for a FIT-Ig binding protein, an Fc region is a human Fc region from IgG1 with at least one Fc effector function (for example the binding of the Fc to FcγR, ADCC and/or CDC) reduced or eliminated, for example, by introduction of LALA mutations (Leu234 to Ala234, Leu235 to Ala235, according to EU numbering system). In a further embodiment, the amino acid sequence of the Fc region is at least 80%, at least 85%, at least 90%, at least 95%, at least 99%, or 100% identical to SEQ ID NO: 40. In one embodiment, the amino acid sequence of the Fc region further comprises triple mutation M252Y/S254T/T256E (YTE, numbering according to EU numbering system). In a further embodiment, the amino acid sequence of the Fc region is at least 80%, at least 85%, at least 90%, at least 95%, at least 99%, or 100% identical to SEQ ID NO: 41.

SEQ ID NO: 40:
DKTHTCPPCPAPEAAGGPSVFLFPPKPKDTLMISRTPEVTCVVVDVSHED
PEVKFNWYVDGVEVHNAKTKPREEQYNSTYRVVSVLIVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGK
SEQ ID NO: 41:
DKTHTCPPCPAPEAAGGPSVFLFPPKPKDTLYITREPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGK

In an embodiment, FIT-Ig binding proteins of the present disclosure retain one or more properties of the parental antibodies. In some embodiments, the FIT-Ig retains binding affinity for the target antigens (i.e., PD-L1 and OX40) comparable to that of the parental antibodies, meaning that the binding affinity of the FIT-Ig binding protein for the OX40 and PD-L1 antigen targets does not vary by greater than 10-fold in comparison to the binding affinity of the parental antibodies for their respective target antigens, as measured by surface plasmon resonance or biolayer interferometry.

In one embodiment, a FIT-Ig binding protein of the present disclosure binds OX40 and PD-L1, and is comprised of a first polypeptide chain, a second polypeptide chain, and a third polypeptide chain, wherein:
the first polypeptide chain comprises an amino acid sequence of SEQ ID NO:35, or a sequence having at least 80%, 85%, 90%, 91%, 92%, 93%, 94%, 95%, 96%, 97%, 98%, 99% or more identity therewith,
the second polypeptide chain comprises an amino acid sequence of SEQ ID NO:36, or a sequence having at least 80%, 85%, 90%, 91%, 92%, 93%, 94%, 95%, 96%, 97%, 98%, 99% or more identity therewith, and
the third polypeptide chain comprises an amino acid sequence of SEQ ID NO:37, or a sequence having at least 80%, 85%, 90%, 91%, 92%, 93%, 94%, 95%, 96%, 97%, 98%, 99% or more identity therewith.

In one embodiment, a FIT-Ig binding protein of the present disclosure binds OX40 and PD-L1, and is comprised of a first polypeptide chain comprising, consisting essentially of, or consisting of the sequence of SEQ ID NO:35; a second polypeptide chain comprising, consisting essentially of, or consisting of the sequence of SEQ ID NO:36; and a third polypeptide chain comprising, consisting essentially of, or consisting of the sequence of SEQ ID NO:37.

### Properties of Bispecific Binding Proteins

In one embodiment, a bispecific OX40/PD-L1 FIT-Ig binding protein capable of binding both PD-L1 and OX40 as described herein comprises a humanized OX40 binding site, or a chimeric OX40 binding site, for instance, a humanized OX40 binding site. In one embodiment, the humanized OX40 binding site in the FIT-Ig protein format has a slower off-rate for OX40 binding, relative to the chimeric OX40 binding site in the same FIT-Ig format, which consists of VH and VL pair of SEQ ID NOs: 10 and 18. In a further embodiment, the off-rate ratio of the humanized OX40 binding site relative to the chimeric OX40 binding site is less than 90%, 80%, 70%, 60%, 50%, 40%, 30%, 20%, 15%, 10%, 5%, as measured by surface plasmon resonance or biolayer interferometry. In one embodiment, the off-rate of a FIT-Ig binding protein described herein for OX40 is less than 5×10⁻³ s⁻¹, less than 3×10⁻³ s⁻¹, less than 2×10⁻³ s⁻¹, less than 1×10⁻³ s⁻¹, less than 9×10⁻⁴ s⁻¹, less than 6×10⁻⁴ s⁻¹, less than 3×10⁻⁴ s⁻¹, less than 2.5×10⁻⁴ s⁻¹, less than 2×10⁻⁴ s⁻¹, less than 1×10⁻⁴ s⁻¹, less than 8×10⁻⁵ s⁻¹, less than 5×10⁻⁵ s⁻¹, as measured by surface plasmon resonance or biolayer interferometry. In one embodiment, a FIT-Ig binding protein antibody described herein or antigen-binding fragment thereof has a dissociation constant (K_{D}) to OX40 in the 10⁻⁸ to 10⁻¹⁰ range, for example, less than 8×10⁻⁸ M, less than 5×10⁻⁸ M, less than 3×10⁻⁸ M, less than 2×10⁻⁸ M, less than 1×10⁻⁸ M, less than 8×10⁻⁹ M, less than 5×10⁻⁹M, less than 3×10⁻⁹ M, less than 2×10⁻⁹ M, or less than 1×10⁻⁹ M, less than 8×10⁻¹⁰ M, less than 6×10⁻¹⁰ M, less than 4×10⁻¹⁰ M, less than 2×10⁻¹⁰ M, or less than 1×10⁻¹⁰ M. In one embodiment, a FIT-Ig binding protein antibody described herein or antigen-binding fragment thereof has an off-rate in the range of 1×10⁻³ s⁻¹ to 1×10⁻⁴ s⁻¹, for example, less than 5×10⁻⁴ s⁻¹, and a K_{D} in the range of 1×10⁻⁸ s⁻¹ to 1×10⁻⁹ s⁻¹, for example, less than 7×10⁻⁹ s⁻¹, in terms of OX40 binding.

In one embodiment, a bispecific OX40/PD-L1 FIT-Ig binding protein binding protein capable of binding PD-L1 and OX40 as described herein, after a one-step purification from cell culture media using a Protein A affinity chromatography, have a purity of no less than 90% as detected by SEC-HPLC. In one embodiment, the one-step purified binding proteins have a purity of no less than 91%, 92%, 93%, 95%, 97%, 99% as detected by SEC-HPLC.

In one embodiment, a bispecific OX40/PD-L1 FIT-Ig binding protein binding protein as described herein is capable of binding both PD-L1-expressing cells and OX40-expressing cells. In one embodiment, the PD-L1-expressing cells are human PD-L1 transfected CHO cell lines, or tumor cells. In one embodiment, the OX40-expressing cells are OX40-expressing T cells/cell lines, for example, CD8+ T cells, CD4+ T cells, Treg cells, or Jurkat cells.

In one embodiment, as measured by flow cytometry in a cell-based assay, the binding potency of the bispecific FIT-Igbinding protein to the OX40-expressing cells are equivalent to or comparable to the corresponding parental anti-OX40 monoclonal IgG antibody comprising the same VH/VL sequence pairs for OX40 binding as the bispecific FIT-Ig protein. In one embodiment, the binding potency of the bispecific FIT-Igbinding protein to the PD-L1-expressing cells are equivalent to, or comparable to the corresponding parental anti-PD-L1 monoclonal IgG antibody comprising the same VH/VL sequence pairs for PD-L1 binding as the bispecific binding protein, as measured by flow cytometry, such as in an assay described in Examples 3 and 4.

In one embodiment, a bispecific binding protein described herein is capable of modulating a biological function of OX40, PD-L1, or both. In one embodiment, the bispecific OX40/PD-L1 FIT-Ig binding protein binding protein as described herein is capable of activating OX40 signaling in terms of PD-L1 dependence. In one embodiment, the bispecific binding proteins of the present disclosure exhibit activation of T cells by OX40 signal pathway. In one embodiment, a bispecific OX40/PD-L1 FIT-Ig binding protein binding protein as described herein exhibits OX40-activated T cell cytotoxicity towards tumor cells in a PD-L1-dependent way. In one embodiment, the bispecific binding proteins of the present disclosure is used for enhancing the cytokine-secretion activity of T-cells towards tumor cells in a PD-L1-dependent way.

In one embodiment, a bispecific OX40/PD-L1 FIT-Ig binding protein binding protein as described herein exhibits PD-L1-dependent OX40 activation. In one embodiment, the ratio of PD-L1-expressing cells to OX40-expressing T cells is about 1:1. In a further embodiment, the bispecific OX40/PD-L1 binding proteins exhibit OX40 activation in T cell activation in the presence of PD-L1-expressing cells, in comparison to much less OX40 activation in T cells in the absence of PD-L1-expressing cells, and in comparison to much less OX40 activation in T cell activation in the presence of PD-L1-expressing cells induced by the combination of corresponding parental anti-PD-L1 monoclonal IgG antibodies comprising the same VH/VL sequence pairs for PD-L1 binding as the bispecific FIT-Ig proteins and corresponding parental anti-OX40 monoclonal IgG antibodies comprising the same VH/VL sequence pairs for OX40 binding as the bispecific FIT-Ig proteins.

In one embodiment, a bispecific OX40/PD-L1 FIT-Ig binding protein binding protein as described herein results in T cell cytotoxicity or cytokine-secretion activity against tumor cells. In a further embodiment, a bispecific OX40/PD-L1 FIT-Ig binding protein binding protein as described herein enhances anti-tumor immunity and/or hampers tumor immune escape. In another embodiment, a bispecific OX40/PD-L1 FIT-Ig binding protein as described herein exhibits anti-tumor activities, such as reducing tumor burden, inhibiting tumor growth, or suppressing neoplastic cell expansion. In some embodiments, the bispecific OX40/PD-L1 FIT-Ig binding protein is capable of mediating super-clustering. In some embodiments, the bispecific OX40/PD-L1 FIT-Ig binding protein is capable of inducing high-order OX40 clustering. In some embodiments, the bispecific OX40/PD-L1 FIT-Ig binding protein is capable of activating T cells in a conditional PD-L1 dependent manner. In some embodiments, the bispecific OX40/PD-L1 FIT-Ig binding protein is capable of triggering sufficient OX40 signaling through PD-L1 crosslinking, e.g., thereby overcoming a limitation of anti-OX40 monotherapy. In some embodiments, the bispecific OX40/PD-L1 FIT-Ig binding protein synergistically stimulates T cell activity, e.g., as measured by methods known in the art such as IL-2 production, as compared to a suitable control, e.g., the additive effect of the combination of both parental antibodies.

### Nucleic Acid, Vector, and Host Cells

In a further aspect, this disclosure provides isolated nucleic acids encoding one or more amino acid sequences of an anti-OX40 antibody of this disclosure or an antigen-binding fragment thereof; isolated nucleic acids encoding one or more amino acid sequences of an anti-PD-L1 antibody of this disclosure or an antigen-binding fragment thereof; and isolated nucleic acids encoding one or more amino acid sequences of a bispecific binding protein, including Fabs-in-Tandem immunoglobulin (FIT-Ig) binding protein, capable of binding both OX40 and PD-L1. Such nucleic acids may be inserted into a vector for carrying out various genetic analyses or for expressing, characterizing, or improving one or more properties of an antibody or binding protein described herein. A vector may comprise one or more nucleic acid molecules encoding one or more amino acid sequences of an antibody or binding protein described herein in which the one or more nucleic acid molecules is operably linked to appropriate transcriptional and/or translational sequences that permit expression of the antibody or binding protein in a particular host cell carrying the vector. Examples of vectors for cloning or expressing nucleic acids encoding amino acid sequences of binding proteins described herein include, but are not limited to, pcDNA, pTT, pTT3, pEFBOS, pBV, pJV, and pBJ, and derivatives thereof.

The present disclosure also provides a host cell expressing, or capable of expressing, a vector comprising a nucleic acid encoding one or more amino acid sequences of an antibody or binding protein described herein. Host cells useful in the present disclosure may be prokaryotic or eukaryotic. An exemplary prokaryotic host cell is Escherichia coli. Eukaryotic cells useful as host cells in the present disclosure include protist cells, animal cells, plant cells, and fungal cells. An exemplary fungal cell is a yeast cell, including Saccharomyces cerevisiae. An exemplary animal cell useful as a host cell according to the present disclosure includes, but is not limited to, a mammalian cell, an avian cell, and an insect cell. Exemplary mammalian cells include, but are not limited to, CHO cells, HEK cells, Jurkat cells, and COS cells.

### Methods for Production

In another aspect, the present disclosure provides a method of producing an anti-OX40 antibody or an antigen binding fragment thereof comprising culturing a host cell comprising an expression vector encoding the antibody or antigen binding fragment in culture medium under conditions sufficient to cause the host cell to express the antibody or fragment capable of binding OX40.

In another aspect, the present disclosure provides a method of producing an anti-PD-L1 antibody or an antigen binding fragment thereof comprising culturing a host cell comprising an expression vector encoding the antibody or antigen binding fragment in culture medium under conditions sufficient to cause the host cell to express the antibody or fragment capable of binding PD-L1.

In another aspect, the present disclosure provides a method of producing a bispecific, multivalent binding protein capable of binding OX40 and PD-L1, specifically a FIT-Ig binding protein binding OX40 and PD-L1, comprising culturing a host cell comprising an expression vector encoding the FIT-Ig binding protein in culture medium under conditions sufficient to cause the host cell to express the binding protein capable of binding OX40 and PD-L1. The proteins produced by the methods disclosed herein can be isolated and used in various compositions and methods described herein.

### Uses of Antibodies and Binding Proteins

Given their ability to bind to human OX40 and/or PD-L1, the antibodies described herein, antigen binding fragments thereof, and bispecific multivalent binding proteins described herein can be used to detect OX40 or PD-L1, or both, e.g., in a biological sample containing cells that express one or both of those target antigens. The antibodies, antigen binding fragments, and binding proteins of the present disclosure can be used in a conventional immunoassay, such as an enzyme linked immunosorbent assay (ELISA), a radioimmunoassay (RIA), or tissue immunohistochemistry. The present disclosure provides a method for detecting OX40 or PD-L1 in a biological sample comprising contacting a biological sample with an antibody, antigen-binding portion thereof, or binding protein of the present disclosure and detecting whether binding to a target antigen occurs, thereby detecting the presence or absence of the target in the biological sample. The antibody, antigen binding fragment, or binding protein may be directly or indirectly labeled with a detectable substance to facilitate detection of the bound or unbound antibody/fragment/binding protein. Suitable detectable substances include various enzymes, prosthetic groups, fluorescent materials, luminescent materials, and radioactive materials. Examples of suitable enzymes include horseradish peroxidase, alkaline phosphatase, β-galactosidase, or acetylcholinesterase. Examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm.

In some embodiments, the antibodies, antigen binding fragments thereof, of the present disclosure are capable of neutralizing human PD-L1 activity both in vitro and in vivo. Accordingly, the antibodies, antigen binding fragments thereof, of the present disclosure can be used to inhibit human PD-L1 activity, e.g., inhibit cell signaling associated with PD-L1 in a cell culture containing PD-L1-expressing cells, in human subjects, or in other mammalian subjects having PD-L1 with which an antibody, antigen binding fragment thereof, or binding protein of the present disclosure cross-reacts.

In another embodiment, the present disclosure provides an antibody or bispecific binding protein of the present disclosure for use in treating a subject suffering from a disease or disorder in which PD-L1 activity is detrimental, wherein the antibody or binding protein is administered to the subject such that activity mediated by PD-L1 in the subject is reduced. As used herein, the term “a disorder in which PD-L1 activity is detrimental” is intended to include diseases and other disorders in which the interaction of PD-L1 with its receptor (for example, PD-1) in a subject suffering from the disorder is either responsible for the pathophysiology of the disorder or is a factor that contributes to a worsening of the disorder. Examples of such diseases or disorders is tumor associated with immune escape, or tumors exhibiting tumor immune escape. Accordingly, a disorder in which PD-L1 activity is detrimental is a disorder in which inhibition of PD-L1 activity is expected to alleviate the symptoms and/or progression of the disorder. In one embodiment, an anti-PD-L1 antibody, antigen binding fragment thereof, or bispecific binding protein of the present disclosure is used in a method that inhibits the growth or survival of malignant cells, or reduces the tumor burden.

In some embodiments, the bispecific binding proteins (FIT-Ig) of the present disclosure are capable of enhancing T cell cytotoxicity or cytokine-secretion activity towards PD-L1-expressing tumor cells both in vitro and in vivo. Accordingly, the bispecific binding proteins of the present disclosure can be used to inhibit the growth or expansion of PD-L1-expressing malignant cells, in human subjects, or in other mammalian subjects having PD-L1 with which an antibody, antigen binding fragment thereof, or bispecific binding protein of the present disclosure cross-reacts.

In another embodiment, the present disclosure provides an antibody or bispecific binding protein of the present disclosure for use in treating a subject suffering from a disease or disorder in which OX40-mediated signaling activity is advantageous (such as OX40⁺ T-cells infiltrated tumors). The term “a disorder in which OX40-mediated signaling activity is advantageous” herein is intended to include diseases and other disorders in which the super-clustering and/or activation of OX40 in a subject suffering from the disorder would thus activate T cells and reverse the effects/alleviate the symptoms/slow down the progression of the disease or disorder, such as tumors. In one embodiment, an anti-OX40 antibody, antigen binding fragment thereof, or bispecific binding protein of the present disclosure is used in a method that inhibits the growth or survival of malignant cells, or reduces the tumor burden.

In another embodiment, the present disclosure provides a PD-L1/OX40 bispecific (FIT-Ig) binding protein for use in treating an PD-L1-expressing malignancy in a subject through the OX40 activation of T cells, wherein the binding protein is administered to the subject. In some embodiments, the malignancy is a tumor, for example, a solid tumor, such as a colon cancer. In some further embodiments, the antibodies (including antigen binding fragments thereof) and binding proteins of the present disclosure are used in the incorporation into, or the manufacture of pharmaceutical compositions suitable for administration to a subject (described supra). Typically, the pharmaceutical composition comprises an antibody or binding protein of the present disclosure and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols (such as, mannitol or sorbitol), or sodium chloride in the composition. Pharmaceutically acceptable carriers may further comprise minor amounts of auxiliary substances such as wetting or emulsifying agents, preservatives, or buffers, which enhance the shelf life or effectiveness of the antibody or binding protein present in the composition. A pharmaceutical composition of the present disclosure is formulated to be compatible with its intended route of administration.

The method of the present disclosure may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the primary active ingredient may be in powder form for constitution with a suitable vehicle (e.g., sterile pyrogen-free water) before use.

The use of the present disclosure may include administration of compositions formulated as depot preparations. Such long-acting formulations may be administered by implantation (e.g., subcutaneously or intramuscularly) or by intramuscular injection. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

An antibody, antigen binding fragment thereof, or binding protein of the present disclosure also can be administered with one or more additional therapeutic agents useful in the treatment of various diseases. Antibodies, antigen binding fragments thereof, and binding proteins described herein can be used alone or in combination with an additional agent, e.g., an additional therapeutic agent, the additional agent being selected by the skilled artisan for its intended purpose. For example, the additional agent can be a therapeutic agent recognized in the art as being useful to treat the disease or condition being treated by the antibody or binding protein of the present disclosure. The additional agent also can be an agent that imparts a beneficial attribute to the therapeutic composition, e.g., an agent that affects the viscosity of the composition.

### Pharmaceutical Compositions

The present disclosure also provides pharmaceutical compositions comprising an antibody, or antigen-binding portion thereof, or a bispecific multivalent binding protein of the present disclosure (i.e., the primary active ingredient) and a pharmaceutically acceptable carrier. In another embodiment, a pharmaceutical composition of the present disclosure may comprise two or more antibodies of the present disclosure, such as, for example, anti-OX40 antibody and anti-PD-L1 antibody. In a further embodiment, a pharmaceutical composition of the present disclosure may comprise at least one antibody, and at least one bispecific binding proteins, according to the present disclosure. In a specific embodiment, a composition comprises one or more antibodies or binding proteins of the present disclosure. The present disclosure also provides pharmaceutical compositions comprising a combination of antibodies (such as, for example, anti-OX40 and anti-PD-L1 antibodies) as described herein, or antigen-binding fragment(s) thereof, and a pharmaceutically acceptable carrier. In particular, the present disclosure provides pharmaceutical compositions comprising at least one FIT-Ig binding protein capable of binding OX40 and PD-L1 and a pharmaceutically acceptable carrier. Pharmaceutical compositions of the present disclosure may further comprise at least one additional active ingredient. In some embodiments, such an additional ingredient includes, but is not limited to, a prophylactic and/or therapeutic agent, a detection agent, such as an anti-tumor drug, a cytotoxic agent, an antibody of different specificity or antigen binding fragment thereof, a detectable label or reporter. In an embodiment, the pharmaceutical composition comprises one or more additional prophylactic or therapeutic agents, i.e., agents other than the antibodies or binding proteins of the present disclosure, for treating a disorder in which PD-L1 activity is detrimental and/or OX40 activity is advantageous. In an embodiment, the additional prophylactic or therapeutic agents are known to be useful for, have been used, or are currently being used in the prevention, treatment, management, or amelioration of, a disorder or one or more symptoms thereof.

The pharmaceutical compositions comprising proteins of the present disclosure are for use in, but not limited to, diagnosing, detecting, or monitoring a disorder; treating, managing, or ameliorating a disorder or one or more symptoms thereof; and/or research. In some embodiments, the composition may further comprise a carrier, diluent, or excipient. An excipient is generally any compound or combination of compounds that provides a desired feature to a composition other than that of the primary active ingredient (i.e., other than an antibody, antigen binding portion thereof, or binding protein of the present disclosure).

### Methods for Treatment and Medical Uses

In one embodiment, the present disclosure provides a method of modulating an immune response in a subject, wherein said method comprising administering to the subject at least one antibody and/or at least one bispecific binding protein according to the present disclosure.

In some embodiments, the present disclosure provides a method for activating T cells. In some further embodiments, the activation of T cells may result in the induction and/or enhancement of T cell mediated antitumor activity. In some further embodiments, the antitumor activity is cytotoxicity and/or cytokine production against tumor cells, wherein said cytokine is, for example, IL-2 or IFN-γ. In some further embodiments, T cells are the CD8+ T cells. In some other embodiments, T cells are the CD4+ T cells. In some embodiments, T cells are effector T cells.

In some embodiments, the present disclosure provides a method for treating cancer in a subject, comprising administering to the subject at least one antibody and/or at least one bispecific binding protein according to the present disclosure. In some embodiments, the cancer is a tumor immune escape, or a tumor exhibiting tumor immune escape. In some further embodiments, the cancer is a cancer that respond to T cell activation, such as cancer with T cell dysfunction. In some further embodiments, the cancer is a cancer having increased level of PD-L1 protein expression or increased level of nucleic acid encoding PD-L1, e.g., compared to the level in the normal subject or the normal cell. In one embodiment, the present disclosure provides methods for treating a disorder in which OX40-mediated signaling activity is advantageous (such as OX40⁺ T-cells infiltrated tumors) in a subject in need thereof, the method comprising administering to the subject an anti-OX40 antibody or OX40-binding fragment thereof as described herein, wherein the antibody or binding fragment is capable of binding OX40 and activating OX40-mediated signaling in a cell expressing OX40. In another embodiment, the present disclosure provides use of an effective amount of an anti-OX40 antibody or antigen-binding fragment thereof described herein in the treatment of such a disorder. In another embodiment, the present disclosure provides use of an anti-OX40 antibody or antigen-binding fragment thereof described herein in the manufacture of a composition for the treatment of such a disorder. In another embodiment, the present disclosure provides an anti-OX40 antibody or antigen-binding fragment thereof described herein for use in the treatment of such a disorder. In a further embodiment of the method or use described herein, an anti-OX40 antibody or antigen binding fragment of the present disclosure binds OX40, and comprises a VH domain comprising, consisting essentially of, or consisting of the sequence of SEQ ID NO: 16, and a VL domain comprising, consisting essentially of, or consisting of the sequence of SEQ ID NO: 21.

In some embodiments, the present disclosure provides methods for treating a disorder in which PD-L1 activity is detrimental in a subject in need thereof, the method comprising administering to the subject an anti-PD-L1 antibody or PD-L1-binding fragment thereof as described herein, wherein the antibody or binding fragment is capable of binding PD-L1 and blocking PD-L1 from the interaction with the receptor of PD-L1, such as, for example, PD-1, and therefore capable of inhibit PD-L1-related signaling in a cell expressing the receptor of PD-L1.

In a further embodiment of the method or use described herein, an anti-PD-L1 antibody or antigen binding fragment of the present disclosure binds PD-L1, and comprises a VH domain comprising, consisting essentially of, or consisting of the sequence of SEQ ID NO: 31, and a VL domain comprising, consisting essentially of, or consisting of the sequence of SEQ ID NO: 34.

In another embodiment, the present disclosure provides methods for treating a disorder in which OX40-mediated signaling activity is advantageous (such as OX40⁺ T-cells infiltrated tumors) and/or PD-L1 activity is detrimental in a subject in need thereof, the method comprising administering to the subject a bispecific FIT-Ig binding protein capable of binding PD-L1 and OX40 as described herein. In another embodiment, the present disclosure provides use of an effective amount of the bispecific FIT-Ig binding protein thereof described herein in the treatment of such a disorder. In another embodiment, the present disclosure provides use of the bispecific FIT-Ig binding protein thereof described herein in the manufacture of a composition for the treatment of such a disorder. In another embodiment, the present disclosure provides the bispecific FIT-Ig binding protein thereof described herein for use in the treatment of such a disorder.

In a further embodiment of the method or use described herein, a FIT-Ig binding protein of the present disclosure binds OX40 and PD-L1 and is comprised of a first polypeptide chain comprising, consisting essentially of, or consisting of the sequence of SEQ ID NO:35; a second polypeptide chain comprising, consisting essentially of, or consisting of the sequence of SEQ ID NO: 36; and a third polypeptide chain comprising, consisting essentially of, or consisting of the sequence of SEQ ID NO:37.

In some embodiments, the disorders which can be treated with the antibody or binding protein according to the present disclosure include various malignancies expressing PD-L1 on the cell surface of the malignant cells. In some further embodiments, the disorders which can be treated with the antibody or binding protein according to the present disclosure include tumors exhibiting tumor immune escape, for example, via PD-L1/PD-1 interaction. In another embodiment, the antibody or the binding protein inhibits the growth or survival of malignant cells. In another embodiment, the antibody or the binding protein reduces the tumor burden. In another embodiment, the cancer is a colon cancer.

Methods of treatment described herein may further comprise administering to a subject in need thereof, of additional active ingredient, which is suitably present in combination with the present antibody or binding protein for the treatment purpose intended, for example, another drug having ant-tumor activity. In a method of treatment of the present disclosure, the additional active ingredient may be incorporated into a composition comprising an antibody or binding protein of the present disclosure, and the composition administered to a subject in need of treatment. In another embodiment, a method of treatment of the present disclosure may comprise a step of administering to a subject in need of treatment an antibody or binding protein described herein and a separate step of administering the additional active ingredient to the subject before, concurrently, or after the step of administering to the subject an antibody or binding protein of the present disclosure.

Having now described the present disclosure in detail, the same will be more clearly understood by reference to the following examples, which are included for purposes of illustration only and are not intended to be limiting of the present disclosure.

### EXAMPLES

### Example 1 Generation of Anti-OX40 Antibody

### Example 1.1 Screening, Cloning and Sequence Analysis of Anti-OX40 Monoclonal Antibody 8G9D5C5

Anti-OX40 monoclonal antibodies were generated by standard hybridoma screening protocols. Cell immunization and Gene gun (DNA immunization) were utilized for immunization, where the immunogens were HEK293 cells overexpressing human OX40, and expression plasmids containing human OX40 gene, respectively. Hybridoma clones were screened for binding activity and biological activity, using human OX40 expressing CHO-K1 cells. Clone 8G9D5C5 was selected for further characterization.

To amplify heavy and light chain variable region of antibody, total RNA of clone 8G9D5C5 was isolated from >5×10⁶ cells with TRIzol reagent (Cat. No. 15596, Invitrogen), and subject to reverse transcription using SuperScript™ III First-Strand Synthesis SuperMix (18080, Invitrogen) to produce cDNA, which was applied as template in PCR using Mouse Ig-Primer Set (Cat. No. 69831-3, Novagen). PCR products were analyzed by electrophoresis on a 1.2% agarose gel with SYBR Safe DNA gel stain. DNA fragments with correct size were purified with NucleoSpin® Gel and PCR Clean-up (Cat. No. 740609, MACHEREY-NAGEL), subcloned into pMD18-T vector individually, then transformed to competent E. coli cells. Fifteen colonies from each transformation were selected and sequences of inserted fragments were analyzed by DNA sequencing. Sequences were confirmed if majority of sequenced colonies (at least 8 out of 15) yielded the same sequence. Amino acid sequences of clone 8G9D5C5 variable region were listed in Table 1. Complement determinant regions (CDRs) were underlined according to Kabat numbering system.

TABLE 1
Amino acid sequences of variable regions
of anti-OX40 antibody

| | | SEQ | |
|---|---|---|---|
| Ab | chain | ID NO. | Amino acid sequences |
| 8G9D | VH | 9 | QVQLQQSGPELVKPGASVTISCKASGHAFSSSWM |
| 5C5 | | | NWVKQRPGKGLEWIGRIYPGDEITNYNGKFKDKA |
| | | | TLTADKSSSTAYMQLSSLTFEDSAVYFCARDLLM |
| | | | PYWGQGTLVTVSA |
| | VL | 17 | DIVMTQTAFSNPVTLGTSASISCRSSKSLLYSNG |
| | | | ITYLYWYLQKPGQSPHLLIYQMSNLAPGVPDRFS |
| | | | SSGSGTDFTLRISRVEAEDVGIYYCAQNLELPFT |
| | | | FGSGTKLEIK |

### Example 1.2 Chimeric Antibody Generation and Characterization

VH and VK genes of 8G9D5C5 as provided in Table 1 were respectively synthesized and cloned into vectors containing the human IgG1 and human kappa constant domains. 293E cells co-transfected with both heavy chain vector and light chain vector were cultured 7 days, then the supernatant was harvested and purified by Protein A chromatography.

The purified chimeric antibody was designated EM1007-44c. Binding activity to human or Cyno OX40 on the cell surface was assessed by FACS. Briefly, 5×10⁵ cells were seeded into each well of a 96-well plate (Corning, #3799). Cells were centrifuged at 400 g for 5 minutes and supernatants were discarded. For each well, 100 μl of 3× serial dilution of antibody starting from 100 nM was then added and mixed with the cells. After 60 minutes of 4° C. incubation, plates were washed to remove excess antibody. Secondary Alexa Fluor® 647-conjugated goat anti-human IgG antibody (1:500 fresh dilution, Jackson ImmunoResearch, #109-606-098) was then added and incubated with cells at room temperature for 20 minutes. After another round of centrifugation and washing, cells were resuspended in FACS buffer for reading on a CytoFLEX Flow Cytometer (Beckman Coulter). Median Fluorescence Intensity (MFI) readouts were plotted against antibody concentration and analyzed with GraphPad Prism 8.0. The ability of OX40 to activate downstream signaling was detected in a Jurkat-OX40-NF-κB luciferase assay. Briefly, high binding plates (Corning, #3361) were coated with 3× serial dilution of EM1007-44c starting from 100 nM at 4° C. overnight, washed, then seeded with 1×10⁵ cells per well of OX40-NF-κB reporter and incubate at 37° C. for 6 hours. At the end of incubation, ONE-Glo™ luminescence assay kit (Promega, Cat. #E6130) reagents were prepared and added according to the manufacturer's instructions. Plates were read for luminescence signals on Varioskan™ LUX microplate reader (ThermoFisher Scientific).

EM1007-44c was further assessed for its ability to activate primary T-cell and promote T-cell proliferation. Briefly, primary T cell stimulation was measured in high binding plates (Corning, #3361) co-coated with 3× serial dilution of EM1007-44c starting from 100 nM and 1 μg/ml OKT3 (Biolegend, #317326) by overnight incubation at 4° C. PD-L1+ T cells were purified from human PBMC with a commercial human T cells isolation kit (Stemcell Technologies, #17951) and added at 1×10⁵ cells per well into the freshly coated and PBS washed plates. The plates were incubated at 37° C. and 5% CO2 for 96 h. For each well, 100 ul supernatant was collected for IFN-γ quantification, then 50 ul/well CellTiter-Glo® Luminescent Cell Viability Assay (Promega, #G7570) mix was added and incubated at RT for 10 minutes for cell viability detection according to manufacturer's instruction.

The data summarized in

Table 2 demonstrate that EM1007-44c has similar binding activity of human OX40 and cyno OX40 on cell surface, and is capable of activating OX40 signaling and primary T-cell.

TABLE 2
Characterization of EM1007-44c

| Characterization Assays | Results |
|---|---|
| Binding to CHO-K1-hOX40, EC50 (nM) | 2.6 |
| Binding to CHO-K1-cOX40, EC50 (nM) | 2.11 |
| T cell activation assay, IFN-γ secretion, EC50 (nM) | 1.18 |
| T cell activation CTG, EC50 (nM) | 3.7 |
| OX40-Jurkat-NF-κB-Reporter, EC50 (nM) | 0.43 |
| OX40-Jurkat-NF-κB-Reporter, Maximal fold change | 1.517 |

### Example 1.3 Humanization of EM1007-44c

The EM1007-mAb044c variable region genes provided in Table 1 were employed to create a humanized antibody. First, amino acid sequences of the VH domain and the VK (VL kappa) domain of EM1007-mAb044c were compared against available human Ig V-gene sequences from V BASE database (https://www2.mrc-lmb.cam.ac.uk/vbase/alignments2.php) in order to find the overall best-matching human germline Ig V-gene sequences. The framework segments of VH and VK were also compared against available FR sequences in the J-region sequences in V BASE to find the human framework having the highest homology to the murine VH and VK regions, respectively. For the light chain, the closest human V-gene match was the O1 gene; and for the heavy chain, the closest human match was the VH1-69 gene. Humanized variable domain sequences were then designed to have the CDR-L1, CDR-L2, and CDR-L3 of the EM1007-044c light chain grafted onto framework sequences of the O1 gene and JK2 framework 4 sequence while the CDR-H1, CDR-H2, and CDR-H3 of the EM1007-mAb044c heavy chain grafted onto framework sequences of the VH1-69 and JH1 framework 4 sequence.

Meanwhile, a three-dimensional Fv model of EM1007-mAb044c was generated to identify any framework positions where mouse amino acids were critical to support loop structures or the VH/VK interface. Corresponding residues within the human framework sequences should be back-mutated to the mouse residues at such identified positions to retain affinity/activity. Several desirable back-mutations were indicated for EM1007-mAb044c VH and VK, and alternative VH and VK designs were constructed as shown in Table 3 below. Since the “NG” (Asn-Gly) pattern found in the CDR-H2 and CDR-L1 of EM1007-mAb044c is prone to deamination and may result in heterogeneity during manufacturing, VH and VL domains containing an NG (Asn-Gly) to NA (Asn-Ala) mutation (highlighted in bold italic), e.g., designated “EM1007-mAb044VH (G-A)” and “EM1007-mAb044VK (G-A)”, were also designed and evaluated.

TABLE 3
Humanized VH/VK Designs for EM1007-044
with Back Mutations*

| Humanized EM1007-mAb044 | Amino Acid Sequence |
|---|---|
| with VH or VK Identifier | 1234567890123456789012345678901234567890 |
| EM1007-mAb044VH | QVQLQQSGPELVKPGASVTISCKASGHAFSSSWMNWVKQR |
| | PGKGLEWIGRIYPGDEITNYNGKFKDKATLTADKSSSTAY |
| | MQLSSLTFEDSAVYFCARDLLMPYWGQGTLVTVSA |
| | (SEQ ID NO: 9) |
| EM1007-mAb044VH(G-A) | QVQLQQSGPELVKPGASVTISCKASGHAFSSSWMNWVKQR |
| | PGKGLEWIGRIYPGDEITNYNAKFKDKATLTADKSSSTAY |
| | MQLSSLTFEDSAVYFCARDLLMPYWGQGTLVTVSA |
| | (SEQ ID NO: 10) |
| HuEM1007-mAb044VH.1 | EVQLVQSGAEVKKPGSSVKVSCKASGGTFSSSWMNWVRQA |
| | PGQGLEWMGRIYPGDEITNYNGKFKDRVTITADKSTSTAY |
| | MELSSLRSEDTAVYYCARDLLMPYWGQGTLVTVSS |
| | (SEQ ID NO: 11) |
| HuEM1007-mAb044VH.1a | EVQLVQSGAEVKKPGSSVKVSCKASGHTFSSSWMNWVRQA |
| | PGQGLEWMGRIYPGDEITNYNGKFKDRVTITADKSTSTAY |
| | MELSSLRSEDTAVYYCARDLLMPYWGQGTLVTVSS |
| | (SEQ ID NO: 12) |
| HuEM1007-mAb044VH.1b | EVQLVQSGAEVKKPGSSVKVSCKASGHTFSSSWMNWVRQA |
| | PGQGLEWIGRIYPGDEITNYNGKFKDRVTLTADKSTSTAY |
| | MELSSLRSEDTAVYYCARDLLMPYWGQGTLVTVSS |
| | (SEQ ID NO: 13) |
| HuEM1007-mAb044VH.1c | EVQLVQSGAEVKKPGSSVKVSCKASGHTFSSSWMNWVKQA |
| | PGKGLEWIGRIYPGDEITNYNGKFKDKVTLTADKSTSTAY |
| | MELSSLRSEDTAVYYCARDLLMPYWGQGTLVTVSS |
| | (SEQ ID NO: 14) |
| HuEM1007-mAb044VH.1d | EVQLVQSGAEVKKPGSSVKVSCKASGGTFSSSWMNWVRQR |
| | PGKGLEWMGRIYPGDEITNYNAKFKDRVTITADKSTSTA |
| | YMELSSLRSEDTAVYYCARDLLMPYWGQGTLVTVSS |
| | (SEQ ID NO: 15) |
| HuEM1007-mAb044VH.1e | EVQLQQSGAEVKKPGSSVKVSCKASGHAFSSSWMNWVKQR |
| | PGKGLEWIGRIYPGDEITNYNAKFKDKATLTADKSTSTA |
| | YMELSSLRSEDTAVYYCARDLLMPYWGQGTLVTVSS |
| | (SEQ ID NO: 16) |
| EM1007-mAb044VK | DIVMTQTAFSNPVTLGTSASISCRSSKSLLYSNGITYLYW |
| | YLQKPGQSPHLLIYQMSNLAPGVPDRFSSSGSGTDFTLRI |
| | SRVEAEDVGIYYCAQNLELPFTFGSGTKLEIK |
| | (SEQ ID NO: 17) |
| EM1007-mAb044VK(G-A) | DIVMTQTAFSNPVTLGTSASISCRSSKSLLYSNAITYLYW |
| | YLQKPGQSPHLLIYQMSNLAPGVPDRFSSSGSGTDFTLRI |
| | SRVEAEDVGIYYCAQNLELPFTFGSGTKLEIK |
| | (SEQ ID NO: 18) |
| HuEM1007-mAb044VK.1 | DIVMTQTPLSLPVTPGEPASISCRSSKSLLYSNGITYLYW |
| | YLQKPGQSPQLLIYQMSNLAPGVPDRFSGSGSGTDFTLKI |
| | SRVEAEDVGVYYCAQNLELPFTFGQGTKLEIK |
| | (SEQ ID NO: 19) |
| HuEM1007-mAb044VK.1a | DIVMTQTPLSLPVTPGEPASISCRSSKSLLYSNGITYLYW |
| | YLQKPGQSPQLLIYQMSNLAPGVPDRFSSSGSGTDFTLKI |
| | SRVEAEDVGVYYCAQNLELPFTFGQGTKLEIK |
| | (SEQ ID NO: 20) |
| HuEM1007-mAb044VK.1b | DIVMTQTPLSLPVTPGEPASISCRSSKSLLYSNAITYLYW |
| | YLQKPGQSPQLLIYQMSNLAPGVPDRFSSSGSGTDFTLKI |
| | SRVEAEDVGVYYCAQNLELPFTFGQGTKLEIK |
| | (SEQ ID NO: 21) |

*CDR sequences according to the Kabat numbering system single underlined;
framework back-mutations double underlined, NG (Asn-Gly) to NA (Asn-Ala)
mutation in bold italic.

The humanized VH and VK (VL kappa) genes were synthesized and then respectively cloned into vectors containing the human IgG1 heavy chain constant domains with LALA mutation and the human kappa light chain constant domain (sequences shown below).

Amino acid sequence of human IgG1 heavy chain
constant domain with LALA mutation
(SEQ ID NO. 42):
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Amino acid sequence of human kappa light chain
constant domain (SEQ ID NO. 43):
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

Pairing of the humanized VH and the humanized VK chains created 10 humanized antibodies, designated “HuEM1007-044-1” to “HuEM1007-044-8”, “HuEM1007-044-14” and “HuEM1007-044-16” as shown in Table 4 below, along with chimeric antibodies designated “EM1007-mAb044c-9” to “EM1007-mAb044c-13”, “EM1007-mAb044c-15” and “EM1007-mAb044c-17” for evaluation potential impact due to the G-A mutation in CDR-H2 and CDR-L1.

TABLE 4
Anti-OX40 Humanized EM1007-044 Antibodies

| Antibody Identifier | VH Region in Heavy Chain | VK Region in Light Chain |
|---|---|---|
| HuEM1007-044-1 | HuEM1007-mAb044VH.1 | HuEM1007-mAb044VK.1 |
| HuEM1007-044-2 | HuEM1007-mAb044VH.1a | HuEM1007-mAb044VK.1 |
| HuEM1007-044-3 | HuEM1007-mAb044VH.1b | HuEM1007-mAb044VK.1 |
| HuEM1007-044-4 | HuEM1007-mAb044VH.1c | HuEM1007-mAb044VK.1 |
| HuEM1007-044-5 | HuEM1007-mAb044VH.1 | HuEM1007-mAb044VK.1a |
| HuEM1007-044-6 | HuEM1007-mAb044VH.1a | HuEM1007-mAb044VK.1a |
| HuEM1007-044-7 | HuEM1007-mAb044VH.1b | HuEM1007-mAb044VK.1a |
| HuEM1007-044-8 | HuEM1007-mAb044VH.1c | HuEM1007-mAb044VK.1a |
| EM1007-mAb044c-9 | EM1007-mAb044VH(G-A) | EM1007-mAb044VK |
| EM1007-mAb044c-10 | EM1007-mAb044VH | EM1007-mAb044VK(G-A) |
| EM1007-mAb044c-11 | EM1007-mAb044VH(G-A) | EM1007-mAb044VK(G-A) |
| EM1007-mAb044c-12 | EM1007-mAb044VH | HuEM1007-mAb044VK.1 |
| EM1007-mAb044c-13 | HuEM1007-mAb044VH.1 | EM1007-mAb044VK |
| HuEM1007-044-14 | HuEM1007-mAb044VH.1d | HuEM1007-mAb044VK.1b |
| EM1007-mAb044c-15 | HuEM1007-mAb044VH.1d | EM1007-mAb044VK(G-A) |
| HuEM1007-044-16 | HuEM1007-mAb044VH.1e | HuEM1007-mAb044VK.1b |
| EM1007-mAb044c-17 | HuEM1007-mAb044VH.1e | EM1007-mAb044VK(G-A) |

All 17 antibodies in Table 4 were expressed by transient transfection of HEK293 cell, purified by Protein A chromatography, and assayed for and ranked by dissociation rate constant (k_{off}). Briefly, antibodies binding affinities and kinetics were characterized by Octet® RED96 biolayer interferometry (Pall FortéBio LLC). Antibodies were captured by Anti-hIgG Fc Capture (AHC) Biosensors (Pall) at a concentration of 100 nM for 120 seconds. After that, sensors were dipped into running buffer (1× pH 7.2 PBS, 0.05% Tween 20, 0.1% BSA) for 60 seconds to check the baseline, then dipped into recombinant human OX40/His fusion protein (Novoprotein, CB17) at assigned concentration for 200 seconds to measure binding, followed by dipped into running buffer for 600 seconds for dissociation. The assay was conducted in four test groups (as listed in Table 5) all containing the EM1007-044c chimeric antibody as the basis for normalization. The association and dissociation curves were fitted to a 1:1 Langmuir binding model using ForteBio Data Analysis software (Pall) to obtain the off-rate constants as shown in Table 5 below. The off-rate of each antibody was compared to that of the EM1007-mAb044c chimeric antibody in the same test group obtained in parallel to produce the corresponding off-rate ratio, serving as a normalized index. The normalized index of an antibody indicates higher affinity for human OX40.

TABLE 5
Off-Rates (koff) of Humanized and
Chimeric EM1007-044 Antibodies

| Test | | Off-Rate (k_{off}) | |
|---|---|---|---|
| Group | Antibody | (1/sec) | Normalized Index |
| 1 | HuEM1007-044-1 | 1.03 × 10⁻⁰³ | 306% |
| | HuEM1007-044-2 | 9.44 × 10⁻⁰⁴ | 281% |
| | HuEM1007-044-3 | 8.59 × 10⁻⁰⁴ | 255% |
| | HuEM1007-044-5 | 8.18 × 10⁻⁰⁴ | 243% |
| | HuEM1007-044-6 | 1.11 × 10⁻⁰³ | 331% |
| | EM1007-mAb044c | 3.36 × 10⁻⁰⁴ | 100% |
| 2 | HuEM1007-044-7 | 1.09 × 10⁻⁰³ | 343% |
| | HuEM1007-044-8 | 1.01 × 10⁻⁰³ | 317% |
| | EM1007-mAb044c-9 | 3.09 × 10⁻⁰⁴ | 97% |
| | EM1007-mAb044c-10 | 2.99 × 10⁻⁰⁴ | 94% |
| | EM1007-mAb044c-11 | 3.17 × 10⁻⁰⁴ | 100% |
| | EM1007-mAb044c-12 | 8.10 × 10⁻⁰⁴ | 255% |
| | EM1007-mAb044c | 3.18 × 10⁻⁰⁴ | 100% |
| 3 | HuEM1007-044-4 | 1.48 × 10⁻⁰³ | 486% |
| | EM1007-mAb044c-13 | 1.30 × 10⁻⁰³ | 427% |
| | EM1007-mAb044c | 3.05 × 10⁻⁰⁴ | 100% |
| 4 | HuEM1007-044-14 | 9.52 × 10⁻⁰⁴ | 341% |
| | EM1007-mAb044c-15 | 9.49 × 10⁻⁰⁴ | 340% |
| | HuEM1007-044-16 | 3.52 × 10⁻⁰⁴ | 126% |
| | EM1007-mAb044c-17 | 2.56 × 10⁻⁰⁴ | 92% |
| | EM1007-mAb044c | 2.79 × 10⁻⁰⁴ | 100% |

The off-rate of EM1007-mAb044c-11 is similar to that of EM1007-mAb044c, suggesting concurrent NG (Asn-Gly) to NA (Asn-Ala) mutations in VH and VL of the former do not compromise the binding affinity. Therefore, HuEM1007-044-16, the humanized design best retains the affinity while also containing both NG to NA mutations, will be used for bispecific molecule construction.

### Example 1.4 Characterization of Anti-OX40 Antibodies

### Example 1.4.1 Epitope Identification

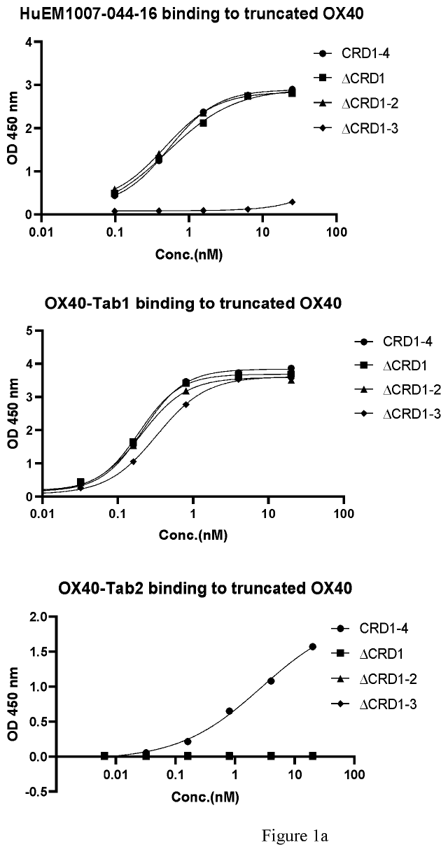

The 4 cysteine rich domains (CRD) from OX40 extracellular domain were identified from UniProt (Identifier: P43489), and full length of extracellular OX40 (CRD1-4) and truncated OX40 variants ΔCRD1 (lacking CRD1), ΔCRD1-2 (lacking CRD1 and CRD2), ΔCRD1-3 (lacking CRD1, CRD2 and CRD3), mCRD1 (CRD1-4 with CRD1 domain therein replaced by murine CRD1), mCRD2 (CRD1-4 with CRD2 domain therein replaced by murine CRD2), mCRD3 (CRD1-4 with CRD3 domain therein replaced by murine CRD3), and mCRD4 (CRD1-4 with CRD4 domain therein replaced by murine CRD4) were synthesized by Biointron. The binding of HuEM1007-044-16, OX40-Tab1 (WO2015153513), OX40-Tab2 (WO2020151761) to OX40 or OX40 truncated proteins were analyzed using ELISA to identify the pertinent binding epitope. Briefly, OX40 variants were each coated at 1 μg/ml onto a 96 well plate (Corning, #3361) which was incubated overnight at 4° C., washed with PBS containing 0.05% Tween 20, blocked with blocking buffer (PBS containing 0.05% Tween 20 and 2% BSA) at 37° C. for 2 hours. On the coated and blocked plate, serially diluted antibodies were added and incubated at 37° C. for 1 hour, washed for 3 times, then added HRP-labeled secondary antibody. Tetramethylbenzidine (TMB) chromogenic solution was added for color development for 5 minutes then the reaction was quench with 1M HCl. Absorbance at 450 nm (OD450) was measured on a microplate reader. FIG. 1a shows results of the ELISA assay described above, suggesting OX40-mAb targets CRD 3 domain, while OX40-Tab1 and OX40-Tab2 respectively target conformational epitope, and CRD1 domain or a conformational epitope containing CRD1 domain. Another ELISA assay was similarly performed except for using OX40 variants with specified CRD domain replaced by the corresponding murine counterpart, result shown in FIG. 1b further suggests human CRD1, CRD2 and CRD4 domains are critical for Tab2 binding.

### Example 1.4.2 Selective Proliferation of T Effector Cells

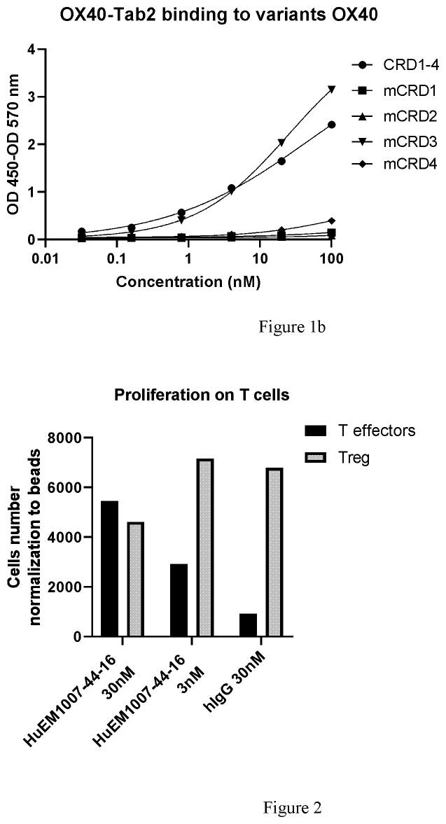

Agonistic effect of anti OX40 antibody was measured by Treg differentiation from CD4 naïve cells. Briefly, CD4 naïve T cells isolated using commercially available kit (Stemcells, #17555) were seeded 5×10⁶ cells/well into a 6-well plate precoated with 2 μg/ml OKT3 and 30 nM of HuEM1007-044-16. After 5 days incubation with supplemented 2 μg/ml CD28 (Biolegend, #302934) and 5 ng/ml TGF-beta, FACS analysis was utilized to assess Treg cell population defined by CD25+Foxp3+, and non-Treg T cell population. FIG. 2 shows HuEM1007-044-16 treatment induced selective proliferation of T effectors over Treg cells, and even reduced Treg polarization at 30 nM concentration.

### Example 1.4.3 OX40 Agonist mAb Internalization Assay

Briefly, 5×10⁵ CHO cells overexpressing human OX40 (CHO-hOX40) can be seeded into each well of a 96-well plate (Corning, #3799) and subject to HuEM1007-044-16 treatment at various concentration with or without internalization inhibitor. After 60 minutes of 37° C. incubation, the plate is washed several times to remove excess antibodies, then added with secondary Alexa Fluor® 647-conjugated goat anti-human IgG antibody (1:500 fresh dilution, Jackson ImmunoResearch, #109-606-098) and incubated at room temperature for 20 minutes. After another round of centrifugation and washing, cells are resuspended in FACS buffer for reading on a CytoFLEX Flow Cytometer (Beckman Coulter). Median Fluorescence Intensity (MFI) readouts can be plotted against antibody concentration and analyzed with GraphPad Prism 8.0.

### Example 2 Generation and Characterization of Anti-PDL1 Antibody

The anti-PD-L1 antibody EM0005-mAb86 was obtained as described in WO2021/104434. After expressed by HEK293 cells and purification by Protein A chromatography, EM0005-mAb86 exhibited an aggregation percentage greater than 10%, which suggests challenge in CMC development of either antibody itself or bispecific molecules utilizing it as a binding domain. For an antibody with better developability, EM0005-mAb86 VH and VL sequence (listed in Table 6) were employed and subjected to framework sequence change for altered physicochemical properties of the full-length antibody, such as change total charge, break hydrophobic patch, and/or increase hydrophilicity without or with minimum impact on the biological activities thereof.

TABLE 6
Amino acid sequence of variable regions
of EM0005-mAb86*

| | | SEQ | |
|---|---|---|---|
| Ab | chain | ID NO. | AA sequences |
| EM0005- | VH | 29 | QVQLQQSGAELVRPGSSVKMSCKTSGYTFTT |
| mAb86 | | | YGINWVKQRPGQGLEWIGYIYIGNGYTEYNE |
| | | | KFKGKATLTSDPSSRTAYMQLSSLTSEDSAI |
| | | | YFCARDLMVIAPKTMDYWGQGTSVTVSS |
| | VL | 32 | DIQMNQSHKFMSTSVGDRVSITCKASQDVGT |
| | | | AVAWYQQKPGQSPKLLIYWASTRHTGVPDRF |
| | | | TGGGSGTDFTLTISNVQSEDLADYFCQQYSS |
| | | | YPYTFGGGTKLEMK |

*CDRs according to Kabat numbering underlined.

In brief, humanized variable domain sequences of EM0005-mAb86 were designed to have its CDR-L1, CDR-L2, and CDR-L3 (as provided in Table 6 for VL of EM0005-mAb86) grafted onto framework sequences of various germline genes from V BASE with JK4 framework 4 sequence after CDR-L3; and its CDR-H1, CDR-H2, and CDR-H3 (as provided in Table 6 for VH of EM0005-mAb86) grafted onto various VH framework sequences from V BASE with JH6 framework 4 sequence after CDR-H3.

The designed VH and VK (VL kappa) genes were synthesized and then respectively cloned into vectors containing the human IgG1 heavy chain constant domains and the human kappa light chain constant domain (sequences provided in Example 1.3). Pairing of the humanized VH and the humanized VK chains created 50 humanized antibodies, 49 of them designated “HuEM0005-86-15” to “HuEM0005-86-63”, along with HuEM0005-86-64, which was designed to have the same sequence as HuEM0005-86-21 except for having a Q (Gln) to E (Glu) mutation at position 1 and a C (Cys) to S (Ser) mutation at position 82a (Kabat numbering). An additional chimeric variant EM0005-86c-1 was designed to have a G55A mutation in CDR-H2 of “EM0005-86c”, to evaluate the impact on antibody binding property by NG (Asn-Gly) to NA (Asn-Ala) mutation, which is assumed desirable for avoiding “NG” (Asn-Gly) in CDR-H2 of EM0005-mAb86, a pattern that is prone to deamination reactions and may result in heterogeneity during manufacturing.

All antibodies were transiently expressed in HEK293, purified by one-step Protein A purification, and evaluated for expression titer and purity by SEC-HPLC. Since the impurity of the purified antibodies is predominantly the aggregation fraction, higher purity indicates lower aggregation propensity of the corresponding antibody.

Based on titer and purity, 10 of the 50 humanized antibodies were selected and further assayed in two groups for dissociation rate constant (k_{off}) as shown in Table 7. Chimeric antibody EM0005-86c having VH/VL sequences identical to EM0005-mAb86 (as provided in Table 6) was used as a positive control in each group and served as a basis for normalization. Briefly, antibodies were characterized for affinities and binding kinetics by Octet® RED96 biolayer interferometry (Pall FortéBio LLC). Anti-hIgG Fc Capture (AHC) Biosensors (Pall) with antibody captured at a concentration of 100 nM for 120 seconds were dipped into running buffer (1× pH 7.2 PBS, 0.05% Tween 20, 0.1% BSA) for 60 seconds to check the baseline, then dipped into a single concentration of recombinant human PD-L1/His fusion protein (Novoprotein, Cat. No. C315) to measure binding for 200 seconds, followed by dipped into running buffer to measure dissociation for 600 seconds. The association and dissociation curves were fitted to a 1:1 Langmuir binding model using FortéBio Data Analysis software (Pall). The off-rate ratios shown in Table 7 were calculated by off-rate of a humanized antibody to that of EM0005-86c in the same test group. The off-rate ratio serves as a normalized index, so that the humanized antibodies can be compared to one another across test groups. The lower off-rate ratio indicates higher affinity of an antibody for human PD-L1. HuEM0005-86-21 was selected for further investigation based on the purity and off-rate data.

TABLE 7
Off-Rates (k_{off}) of Humanized and Chimeric EM0005-mAb86 Antibodies

| | | | Off-Rate | Off-Rate | Purity |
|---|---|---|---|---|---|
| Test | | Productivity | (K_{off}) | Ratio to | by SEC- |
| Group | Antibody | (mg/L) | (1/sec) | EM0005-86c | HPLC |
| 1 | HuEM0005-86-18 | 5.0 | 3.25 × 10⁻⁰⁴ | 119% | 92% |
| | HuEM0005-86-20 | 2.2 | 2.26 × 10⁻⁰⁴ | 83% | 97% |
| | HuEM0005-86-21 | 7.0 | 2.39 × 10⁻⁰⁴ | 88% | 95% |
| | HuEM0005-86-29 | 1.2 | 1.63 × 10⁻⁰⁴ | 60% | 90% |
| | EM0005-86c-1 | 7.7 | 1.88 × 10⁻⁰⁴ | 69% | 91% |
| | EM0005-86c | | 2.72 × 10⁻⁰⁴ | 100% | 90% |
| 2 | HuEM0005-86-39 | 25.4 | 2.22 × 10⁻⁰⁴ | 153% | 91% |
| | HuEM0005-86-41 | 13.4 | 1.57 × 10⁻⁰⁴ | 108% | 96% |
| | HuEM0005-86-42 | 14.0 | 1.41 × 10⁻⁰⁴ | 97% | 95% |
| | HuEM0005-86-55 | 4.7 | 1.71 × 10⁻⁰⁴ | 118% | 90% |
| | HuEM0005-86-62 | 22.4 | 2.47 × 10⁻⁰⁴ | 170% | 94% |
| | HuEM0005-86-63 | 19.0 | 2.76 × 10⁻⁰⁴ | 190% | 94% |
| | EM0005-86c | | 1.46 × 10⁻⁰⁴ | 100% | 90% |

On the basis of HuEM0005-86-21, HuEM0005-86-64 was further designed to include C82aS mutation and used for FIT-Ig construction. The sequences of HuEM0005-86-64 are shown in Table 8.

TABLE 8
Amino acid sequences of HuEM0005-86-64

| | | Amino Acid Sequence |
|---|---|---|
| Identifier | SEQ ID NO | 1234567890123456789012345678901234567890 |
| HuEM0005-86-64 | 31 | EVQLVQSGSELKKPGASVKVSCKASGYTFTTYGINWVRQA |
| VH | | PGQGLEWMGYIYIGNAYTEYNEKFKGRFVFSLDTSVSTAY |
| | | LQISSLKAEDTAVYYCARDLMVIAPKTMDYWGQGTTVTVS |
| | | S |
| HuEM0005-86-64 | 34 | DIQMTQSPSSVSASVGDRVTITCKASQDVGTAVAWYQQKP |
| VL | | GKAPKLLIYWASTRHTGVPSRFSGSGSGTDFTLTISSLQP |
| | | EDFATYYCQQYSSYPYTFGGGTKVEIK |

### Example 3 Generation and Characterization of PDL1/OX40 FIT-Ig

### Example 3.1 Construction of PDL1/OX40 FIT-Ig FIT1014-20a

A PD-L1/OX40 FIT-Ig designated FIT1014-20a was constructed utilizing coding sequences for immunoglobulin domains of the parental antibodies HuEM0005-86-64 (humanized anti-PD-L1, see Table 8) and HuEM1007-44-16 (humanized anti-OX40, see Table 3 and Table 4). FIT-Ig FIT1014-20a is a hexamer comprised of three component polypeptide chains:

Polypeptide chain #1 has the domain formula: VL-CL of HuEM0005-86-64 fused directly to VH-CH1 of HuEM1007-44-16 fused directly to Fc of a mutant human constant IgG1; CH2 domain the triple mutation M252Y/S254T/T256E (‘YTE’, EU numbering) which causes an about 10-fold increase in their binding to the human neonatal Fc receptor (FcRn). This may increase serum half-life of FIT1014-20a.

Polypeptide chain #2 has the domain formula: VH-CH1 of HuEM0005-86-64; and Polypeptide chain #3 has the domain formula: light chain (VL-CL) of HuEM1007-44-16. The amino acid sequences for the three expressed FIT1014-20a polypeptide chains are shown in Table 9 below.

TABLE 9
Amino Acid Sequences of FIT1014-20a Component Chains*

| | | Amino Acid Sequence |
|---|---|---|
| Polypeptide | SEQ ID NO: | 1234567890123456789012345678901234567890 |
| FIT1014-20a | 35 | DIQMTQSPSSVSASVGDRVTITCKASQDVGTAVAWYQQKP |
| FIT-Ig Polypeptide | | GKAPKLLIYWASTRHTGVPSRFSGSGSGTDFTLTISSLQP |
| Chain #1 | | EDFATYYCQQYSSYPYTFGGGTKVEIKRTVAAPSVFIFPP |
| (VL_{PDL1}-CL- | | SDEQLKSGTASVVCLLNNFYPREAKVQWKVDNALQSGNSQ |
| VH_{OX40}-CH1-Fc) | | ESVTEQDSKDSTYSLSSTLTLSKADYEKHKVYACEVTHQG |
| | | LSSPVTKSFNRGECEVQLQQSGAEVKKPGSSVKVSCKASG |
| | | HAFSSSWMNWVKQRPGKGLEWIGRIYPGDEITNYNAKFKD |
| | | KATLTADKSTSTAYMELSSLRSEDTAVYYCARDLLMPYWG |
| | | QGTLVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHTCPP |
| | | CPAPEAAGGPSVFLFPPKPKDTLYITREPEVTCVVVDVSH |
| | | EDPEVKFNWYVDGVEVHNAKTKPREEQYNSTYRVVSVLTV |
| | | LHQDWLNGKEYKCKVSNKALPAPIEKTISKAKGQPREPQV |
| | | YTLPPSREEMTKNQVSLTCLVKGFYPSDIAVEWESNGQPE |
| | | NNYKTTPPVLDSDGSFFLYSKLTVDKSRWQQGNVFSCSVM |
| | | HEALHNHYTQKSLSLSPGK |
| FIT1014-20a | 36 | EVQLVQSGSELKKPGASVKVSCKASGYTFTTYGINWVRQA |
| FIT-Ig Polypeptide | | PGQGLEWMGYIYIGNAYTEYNEKFKGRFVFSLDTSVSTAY |
| Chain #2 | | LQISSLKAEDTAVYYCARDLMVIAPKTMDYWGQGTTVTVS |
| (VHPDL1-CH1) | | SASTKGPSVFPLAPSSKSTSGGTAALGCLVKDYFPEPVTV |
| | | SWNSGALTSGVHTFPAVLQSSGLYSLSSVVTVPSSSLGTQ |
| | | TYICNVNHKPSNTKVDKKVEPKSC |
| FIT1014-20a | 37 | DIVMTQTPLSLPVTPGEPASISCRSSKSLLYSNAITYLYW |
| FIT-Ig Polypeptide | | YLQKPGQSPQLLIYQMSNLAPGVPDRESSSGSGTDFTLKI |
| Chain #3 | | SRVEAEDVGVYYCAQNLELPFTFGQGTKLEIKRTVAAPSV |
| (VL_{OX40}-CL) | | FIFPPSDEQLKSGTASVVCLLNNFYPREAKVQWKVDNALQ |
| | | SGNSQESVTEQDSKDSTYSLSSTLTLSKADYEKHKVYACE |
| | | VTHQGLSSPVTKSFNRGEC |

*Variable regions in bold; YTE mutation underlined.

DNA molecules encoding amino acid sequences for each of the three component polypeptide chains were synthesized and cloned into pcDNA3.1 mammalian expression vectors. The three recombinant pcDNA3.1 expression vectors for expressing each of the three component polypeptide chains were co-transfected into HEK 293E cells. After approximately six days of post-transfection cell culture, the supernatants were harvested and subjected to Protein A affinity chromatography to obtain purified PD-L1/OX40 FIT-Ig bispecific binding protein.

### Example 3.2 Binding Activity of PDL1/OX40 FIT-Ig

### FACS Binding

Cell binding affinity of PD-L1/OX40 antibodies were measured against CHO (ATCC, #CCL-61) overexpressing human PD-L1 (CHO-PD-L1) and CHO overexpressing OX40 (CHO-OX40), respectively. Briefly, 5×10⁵ cells were seeded into each well of a 96-well plate (Corning, #3799). Cells were centrifuged at 400 g for 5 minutes and supernatants were discarded. For each well, 100 μl of 3× serial dilution of antibodies starting from 100 nM were then added and mixed with the cells. After 60 minutes of 4° C. incubation, plates were washed several times to remove excess antibodies. Secondary Alexa Fluor® 647-conjugated goat anti-human IgG antibody (1:500 fresh dilution, Jackson ImmunoResearch, #109-606-098) was then added and incubated with cells at room temperature for 20 minutes. After another round of centrifugation and washing, cells were resuspended in FACS buffer for reading on a CytoFLEX Flow Cytometer (Beckman Coulter). Median Fluorescence Intensity (MFI) readouts were plotted against antibody concentration and analyzed with GraphPad Prism 8.0.

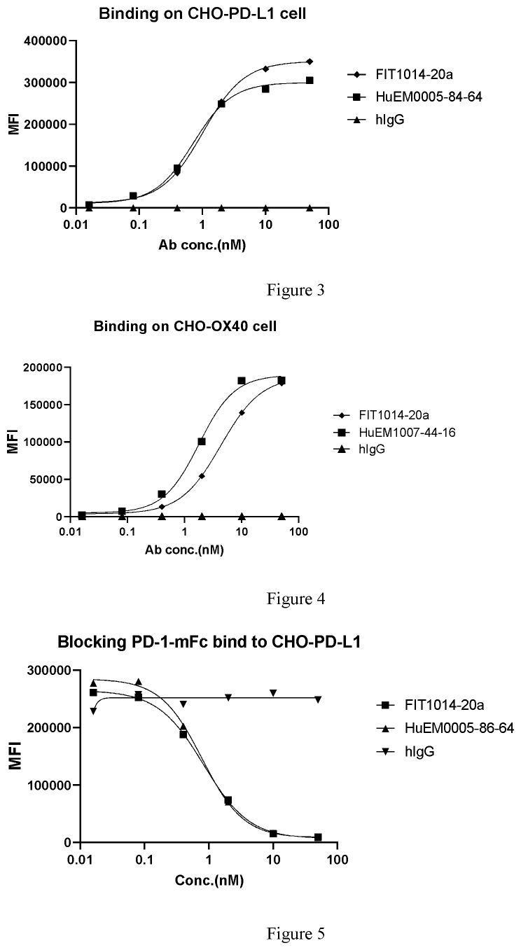

As shown in FIG. 3, the binding affinity to CHO-PD-L1 of FIT-1014-20a is rather close to that of its parental anti-PD-L1 monoclonal antibody (HuEM0005-86-64), and a negative irrelevant human IgG did not show any binding.

The result of FACS affinity for binding to human OX40 transfected CHO cell shown in FIG. 4 indicates FIT1014-20a has a binding affinity relatively lower than that of its parental OX40 antibody HuEM1007-44-16 (EC50 of 4.3 nM vs. 1.8 nM).

### Affinity to PD-L1 and OX40

The PD-L1 binding activities of FIT1014-20a were detected by biolayer interferometry using an Octet® Red sensing device (ForteBio, Red96). Anti-hIgG Fc Capture (AHC) Biosensors (Pall) with FIT1014-20a (YTE) captured at concentration of 100 nM for 30 seconds were dipped into running buffer (1× pH 7.2 PBS, 0.05% Tween 20, 0.1% BSA) for 60 seconds to check baseline, then dipped into serial dilution (100 nM, 33.3 nM, 11.1 nM, 3.7 nM) of recombinant human or cyno PD-L1-his protein to measure binding for 200 seconds, followed by dipped into running buffer to measure dissociation for 1200 seconds. The association and dissociation curves were fitted to a 1:1 Langmuir binding model using FortéBio Data Analysis software (Pall) to produce kinetic rate constants Kon and Koff. The equilibrium dissociation constant KD (M) of the reaction between antibodies and related target proteins was then calculated by KD=koff/kon. The affinity to OX40 was detected similarly except for using human or cyno OX40 (300 nM, 100 nM, 33.3 nM, 11.1 nM,) instead of PD-L1-his protein. The results are shown in Table 10, below.

TABLE 10
Binding affinity of FIT1014-20a to PD-L1 and OX40 proteins

| Analyte | kon(1/Ms) | koff(1/s) | KD (M) |
|---|---|---|---|
| human OX40 | 7.81E+04 | 4.70E−04 | 6.02E−09 |
| Cyno OX40 | 7.80E+04 | 4.00E−04 | 5.13E−09 |
| human PD-L1 | 6.20E+05 | 1.58E−04 | 2.55E−10 |
| Cyno PD-L1 | 4.70E+05 | 2.76E−04 | 5.87E−10 |

### Example 4 PD-L1 Blocking

### Example 4.1 Blocking PD-1/PD-L1 Binding

Blocking of PD-1/PD-L1 binding was assessed in a cell based receptor blocking assay (RBA), 100 μl of 2×10⁵ cells/well of CHO-PD-L1 cells were added to a 96-well round-bottomed plate (Corning, Cat #3799), 50 μl serial diluted of antibody at 0.016 nM to 50 nM and 50 μl of 50 μg/ml PD-1-mFc (Novoprotein, #C754) were added to each well, mixed gently then incubated at 4° C. for 1 hour. The cells were washed and stained by Alexa Fluor® 647 anti-mouse IgG (1:500, Jackson ImmunoResearch, #115-606-008). Signals were read out by FACS and curves were fitted by GraphPad Prism 8.0. As shown in FIG. 5, FIT1014-20a demonstrated a potency of blocking PD-1 protein binding to PD-L1 over-expressing cells similar to that of its parental anti-PD-L1 antibody, HuEM0005-86-64.

### Example 4.2 Blocking PD-L1 Mediated Inhibitory Signaling

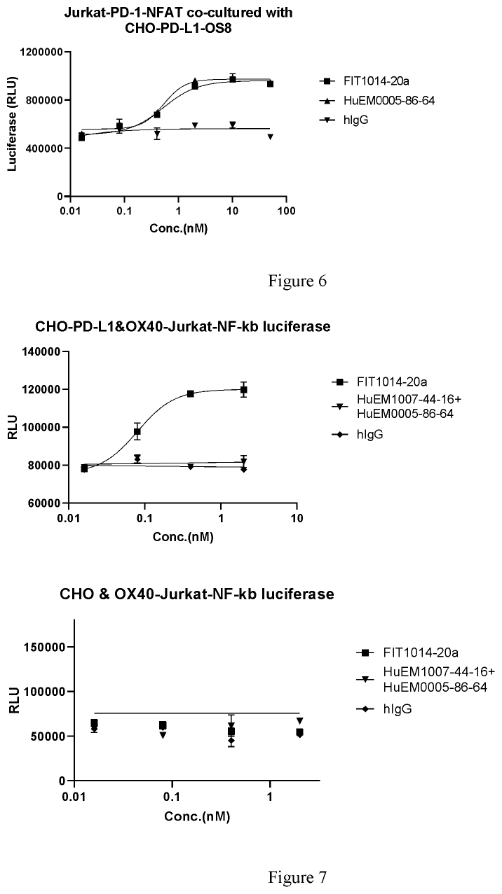

Blocking of PD-L1 inhibitory signaling was examined by coculture of CHO-PD-L1-OS8 (as disclosed in U.S. Pat. No. 8,735,553, with OS8 used as a T cell activating molecule and PD-L1 were stably transduced) and Jurkat-PD-1-NFAT-luciferase reporter cell line expressing both human PD-1 and a luciferase reporter driven by a NFAT response element. Briefly, Logarithmic growth period of CHO-PD-L1-OS8 were harvested, washed, and resuspended in assay medium (RPMI1640 with 10% FBS), and seeded 50 μl for 1×10⁵ cells per well into 96 well plates (Corning, Cat. #3799), then added 50 μl of Jurkat-PD-1-NFAT-luciferase reporter cell for 1×10⁵ per well. 50 ul of serially diluted sample antibodies were added and incubated with the cell mixture for 6 hours at 37° C., After incubation, ONE-Glo™ luminescence assay kit (Promega, Cat. #E6130) reagents were prepared and added according to the manufacturer's instructions. Plates were read for luminescence signals on Varioskan™ LUX microplate reader (ThermoFisher Scientific). As shown in FIG. 6, FIT1014-20a demonstrated similar performance in blocking PD-L1 mediated PD-1 downstream signaling, as did its parental anti-PD-L1 antibody, HuEM0005-86-64.

### Example 5. PD-L1 Dependent Activation of OX40 Downstream Signaling

The ability to induce PD-L1 dependent OX40 downstream signaling activation was assessed by coculturing CHO-PD-L1 with Jurkat-OX40-NFKβ-luciferase reporter cell line followed by examining activation of OX40 through PD-L1 crosslinking. 100 μl of CHO expressing PD-L1 for 4×10⁴ cells/well and 100 μl of OX40-expressing NFKβ-luciferase reporter cell line for 1×10⁵ cells/well were co-seeded into 96 well plates (Corning, Cat. #3799), incubated with 50 μl serially diluted antibodies at 37° C. for 6 hours. At the end of incubation, ONE-Glo™ luminescence assay kit (Promega, Cat. #E6130) reagents were prepared and added according to the manufacturer's instructions. Plates were read for luminescence signals on Varioskan™ LUX microplate reader (ThermoFisher Scientific). As shown in Figure, FIT1014-20a induced activation of OX40 downstream NF-K signaling pathway in a dose-dependent manner at the presence of PD-L1 positive cells (FIG. 7, top), whereas the combination of parental mAb did not. A parallel assay using the same antibodies and reporter cell line but with PD-L1 negative CHO cells was performed as a control, and the lack of activation as shown in FIG. 7 (bottom) indicates the FIT1014-20a induced activation is PD-L1 dependent.

### Example 6. T Cell Activation

### Example 6.1 IFN-γ and IL2 Production by Primary T Cells

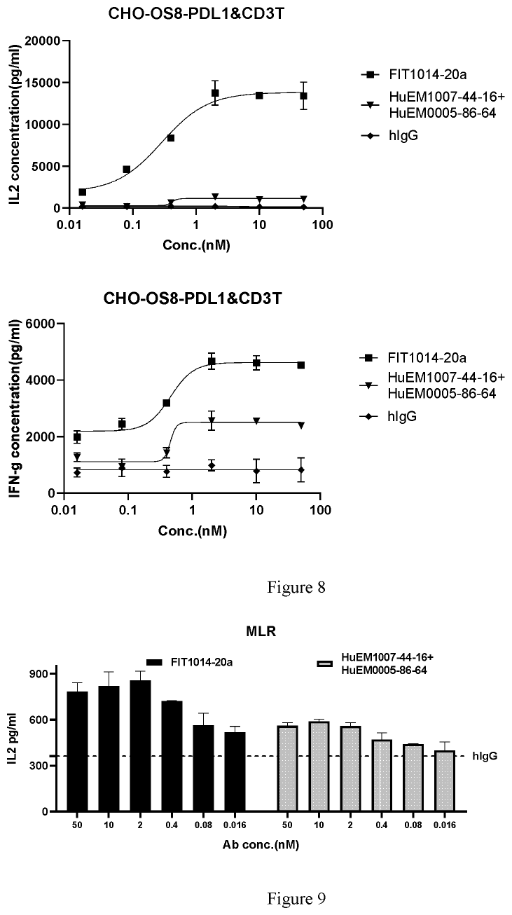

The T cell activation was measured by the IFN-γ and IL2 production in a co-culture system of CHO-PD-L1-OS8 cells and human primary T cells. Briefly, CHO-PD-L1-OS8 were harvested, washed, and resuspended in assay medium (RPMI1640 with 10% FBS) to 4×10⁵ cells/ml, add 100 μl of cells into 96-well plate (Corning, #3799). T cells were purified from human PBMC with a commercial human T cells isolation kit (Stemcell Technologies, #17951) and 100 μl of 4×10⁵ cells/ml were added cells plates. Test antibodies and a negative irrelevant human IgG were added and incubated with the cell mixture for 48 hours at 37° C., the supernatant was sampled for IFN-γ production measurement using a PerkinElmer IFN-γ detection kit (PerkinElmer; #TRF1217M). Further supernatant was sampled after 72 hours of incubation for IL-2 production measurement using a PerkinElmer IL-2 detection kit (PerkinElmer; #TRF1221M). According to IFN-γ and IL2 production shown in FIG. 8, FIT1014-20a could activate T cells to produce both IFN-γ and IL2 in a dose dependent manner as compared to the combination of monospecific parental antibodies.

### Example 6.2 Mixed Lymphocyte Reaction (MLR) Assay

To determine if the bispecific antibody described herein (BsAb) could synergistically stimulate T cells by through simultaneous activation of OX40 and PD-L1/PD-1 blockage, a mixed lymphocyte reaction (MLR) assay was set up as reported (Tourkva et al., 2001) to evaluate the effect on T cell activation. Briefly, monocytes isolated from PBMCs with monocyte enrichment kit (Stemcell, 19058) were maintained in medium (RPMI1640+10% FBS) supplemented with 50 ng/ml GM-CSF (R&D, #215-GM-050/CF), 35 ng/ml IL-4 (R&D, #204-IL-050/CF) for 6 days. Mature DC were induced by supplemented with 20 ng/ml TNF-a (R&D, #210-TA-005/CF), 50 μg/ml Poly I:C (Sigma, #13036) and incubated at 37° C. and 5% CO2 for another 2 days. Allogeneic human CD4⁺ T cells isolated from PBMCs by EasySep™ human CD4⁺ T enrichment kit (Stemcell, #17952) 100 μl of CD4+ T cells were seeded for 1×10⁵ cells per well into 96-well round bottom plates (Corning, #3799), and 100 μl of mature DCs were added into plates for 1×10⁵ cells per well and incubated with serially diluted antibodies. Supernatant was collected after 3 days to detect IL-2 production as the readout of MLR response. IL2 levels from MLR assay shown in FIG. 9 suggests FIT1014-20a is more potent in promoting IL-2 production than the combination of both parental antibodies.

### Example 6.3 Staphylococcal Enterotoxin B (SEB) Assay

A bacterial toxin stimulation assay using superantigen Staphylococcus aureus enterotoxin B (SEB) was conducted for further evaluation of the effect on T cell activation. Briefly, 100 μl of PBMCs from a healthy human donor were seeded into a 96-well assay plate at 2×10⁵ cells/well, then 50 μl of serial dilution of test antibodies was added and incubated with the PBMC at 37° C. for 30 min. 50 μl of SEB solution at final concentration of 10 ng/ml was added and the plate was further incubated for 96 hours. 100 μl of cell culture supernatant were collected for IL-2 measurement using a PerkinElmer IL-2 detection kit (PerkinElmer; #TRF1221M). As indicated by IL-2 level shown in FIG. 10, FIT-1014-20a is more potent in enhancing T cell activation than the combination of its parental anti-OX40 and PD-L1 antibodies.

### Example 7 Investigation of Fc Mediating Effector Function

### Example 7.1 Complement Dependent Cytotoxicity to Activated CD4+ Cells

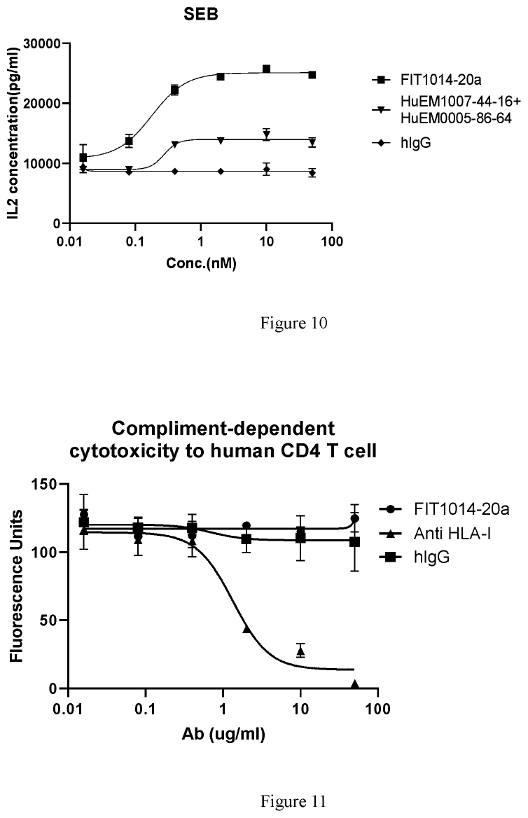

Human CD4⁺ T cells isolated from PBMCs by EasySep™ human CD4⁺ T enrichment kit (Stemcell, #17952), and activated by T cell activator (Stemcell, #10971) for 3 days, activated CD4⁺ T cells were harvested and diluted to 1×10⁶ cells/ml, 50 μL of activated CD4⁺ T cells solution were added into 96 well cell plates (Corning, #3799), 50 μL of normal human serum complement (Quidel, #A113) was added into cell plates, then added with 50 μL serially diluted antibody, irrelevant human IgG (as negative control) or anti HLA (as positive control, AntibodyGenie, AGEL1612). After 6 h incubation at 37° C., 5% CO2, the cell cytotoxicity alamarBlue™ Cell Viability Reagent (Thermofisher, #DAL1100). As shown in FIG. 11, FIT1014-20a did not produce any cytotoxicity to activated human CD4⁺ T cells, while the positive control anti-HLA showed dose dependent cytotoxicity to activated human CD4⁺ T cells.

### Example 7.2 Phagocytosis Effect to CHO-OX40

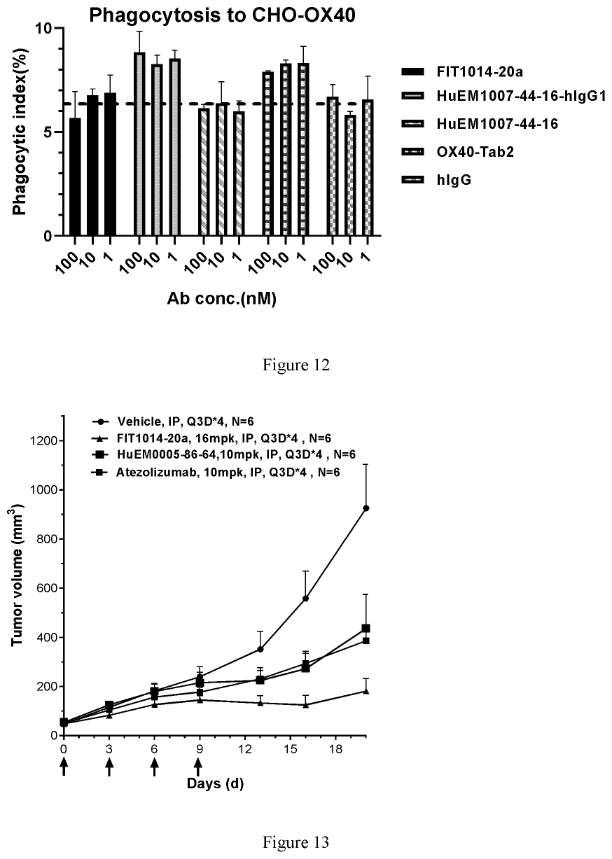

Briefly, CD14+ monocytes were isolated from fresh PBMC, differentiated into macrophage by incubation with 100 ng/ml M-CSF (R&D; 216-MC-010/CF) for 6 days, then labeled with CellTrace™ Far Red (Thermo Fisher Scientific, C34564). Into a 96 well plate (Corning, 7007) was added 100 μL of CellTrace™ CFSE (Thermo Fisher Scientific, C34554) labeled CHO-OX40 cells for 2×10⁵ cell per well, 50 μL of Far red labeled human macrophages at the density of 4×10⁵, and 50 μL of antibodies at assigned concentration, then incubated at 37° C. for 3 h. Phagocytosis was assessed by the percentage of CFSE⁺ cells in Far red⁺ cells via flow cytometry. As shown in FIG. 12, FIT1014-20a and HuEM1007-044-16 (parental OX40 mAb) produced little phagocytosis effect, whereas its parental HuEM1007-044-16 with hIgG1 HuEM1007-044-16 and reference antibody OX40-Tab2 showed some phagocytosis effect.

### Example 8 Anti-Tumor Efficacy in Humanized PD-L1 and OX40 Mice Bearing MC38 Colon Tumor Model

The antitumor efficacy of FIT1014-20a was examined in hPD-L1/hOX40 transgene syngeneic mice model (Biocytogen, Beijing, China) bearing human PD-L1 expressing MC38 tumor cells (Shanghai Model Organisms Center Inc., Shanghai, China). PD-L1-expressing MC38 cells (5×10⁶ cells) suspended in 0.1 ml PBS were injected subcutaneously into the right dorsal flank of hPD-L1/hOX40tg female mice. Five days later (Day 0), mice were randomly assigned to groups (n=6) based on tumor volume (average of 70 mm³). Intraperitoneal injection with test antibodies on Day 0, 3, 6, 9. Tumor dimensions and body weights were measured twice a week. The results in FIG. 13 demonstrates that FIT1014-20a treatment group mice showed tumor growth inhibition superior to monotherapy of Atezolizumab or parental PD-L1 mAb (HuEM0005-86-64).

### Example 9 Anti-Tumor Efficacy in Humanized PD-L1, PD-1 and OX40 Mice Bearing CT26 Colon Tumor Model

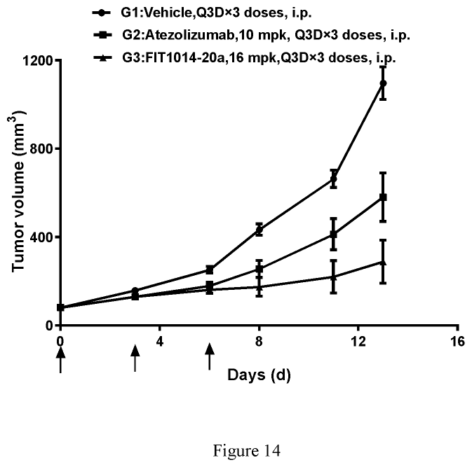

The in vivo anti-tumor activities of FIT1014-20a following i.p. administrations were further investigated against established CT26-hPD-L1 syngeneic tumors in human PD-1/PD-L1/OX40 knock-in mice (GemPharmatech, China). PD-L1-expressing CT26 cells (2.5×10⁶ cells) suspended in 0.1 ml PBS were injected subcutaneously into the right dorsal flank of hPD-L1/hPD-1/hOX40 transgene female mice. Five days later (Day 0), mice were randomly grouped (n=8) based on tumor volume (average of 80 mm³), and subject to intraperitoneal injections of test antibodies on Day 0, 3, 6. Tumor dimensions and body weights were measured twice a week. The result in FIG. 14 demonstrates that FIT1014-20a treatment group mice showed tumor growth inhibition superior to monotherapy of Atezolizumab mAb.

List of sequences:

| SEQ ID NO: | Description | Sequences |
|---|---|---|
| 1 | Anti-OX40 HCDR1 | SSWMN |
| 2 | Anti-OX40 HCDR2 | RIYPGDEITNYNGKFKD |
| 3 | Anti-OX40 HCDR3 | DLLMPY |
| 4 | Anti-OX40 HCDR2 | RIYPGDEITNYNAKFKD |
| 5 | Anti-OX40 LCDR1 | RSSKSLLYSNGITYLY |
| 6 | Anti-OX40 LCDR2 | QMSNLAP |
| 7 | Anti-OX40 LCDR3 | AQNLELPFT |
| 8 | Anti-OX40 LCDR1 | RSSKSLLYSNAITYLY |
| 9 | EM1007-mAb044VH | QVQLQQSGPELVKPGASVTISCKASGHAFSSSWMNWVKQ |
| | (clone.8G9D5C5) | RPGKGLEWIGRIYPGDEITNYNGKFKDKATLTADKSSST |
| | | AYMQLSSLTFEDSAVYFCARDLLMPYWGQGTLVTVSA |
| 10 | EM1007- | QVQLQQSGPELVKPGASVTISCKASGHAFSSSWMNWVKQ |
| | mAb044VH(G-A) | RPGKGLEWIGRIYPGDEITNYNAKFKDKATLTADKSSST |
| | | AYMQLSSLTFEDSAVYFCARDLLMPYWGQGTLVTVSA |
| 11 | HuEM1007- | EVQLVQSGAEVKKPGSSVKVSCKASGGTFSSSWMNWVRQ |
| | mAb044VH.1 | APGQGLEWMGRIYPGDEITNYNGKFKDRVTITADKSTST |
| | | AYMELSSLRSEDTAVYYCARDLLMPYWGQGTLVTVSS |
| 12 | HuEM1007- | EVQLVQSGAEVKKPGSSVKVSCKASGHTFSSSWMNWVRQ |
| | mAb044VH.1a | APGQGLEWMGRIYPGDEITNYNGKFKDRVTITADKSTST |
| | | AYMELSSLRSEDTAVYYCARDLLMPYWGQGTLVTVSS |
| 13 | HuEM1007- | EVQLVQSGAEVKKPGSSVKVSCKASGHTFSSSWMNWVRQ |
| | mAb044VH.1b | APGQGLEWIGRIYPGDEITNYNGKFKDRVTLTADKSTST |
| | | AYMELSSLRSEDTAVYYCARDLLMPYWGQGTLVTVSS |
| 14 | HuEM1007- | EVQLVQSGAEVKKPGSSVKVSCKASGHTFSSSWMNWVKQ |
| | mAb044VH.1c | APGKGLEWIGRIYPGDEITNYNGKFKDKVTLTADKSTST |
| | | AYMELSSLRSEDTAVYYCARDLLMPYWGQGTLVTVSS |
| 15 | HuEM1007- | EVQLVQSGAEVKKPGSSVKVSCKASGGTFSSSWMNWVRQ |
| | mAb044VH.1d | RPGKGLEWMGRIYPGDEITNYNAKFKDRVTITADKSTST |
| | | AYMELSSLRSEDTAVYYCARDLLMPYWGQGTLVTVSS |
| 16 | HuEM1007- | EVQLQQSGAEVKKPGSSVKVSCKASGHAFSSSWMNWVKQ |
| | mAb044VH.1e | RPGKGLEWIGRIYPGDEITNYNAKFKDKATLTADKSTST |
| | | AYMELSSLRSEDTAVYYCARDLLMPYWGQGTLVTVSS |
| 17 | EM1007-mAb044VK | DIVMTQTAFSNPVTLGTSASISCRSSKSLLYSNGITYLY |
| | | WYLQKPGQSPHLLIYQMSNLAPGVPDRFSSSGSGTDFTL |
| | | RISRVEAEDVGIYYCAQNLELPFTFGSGTKLEIK |
| 18 | EM1007- | DIVMTQTAFSNPVTLGTSASISCRSSKSLLYSNAITYLY |
| | mAb044VK(G-A) | WYLQKPGQSPHLLIYQMSNLAPGVPDRFSSSGSGTDFTL |
| | | RISRVEAEDVGIYYCAQNLELPFTFGSGTKLEIK |
| 19 | HuEM1007- | DIVMTQTPLSLPVTPGEPASISCRSSKSLLYSNGITYLY |
| | mAb044VK.1 | WYLQKPGQSPQLLIYQMSNLAPGVPDRFSGSGSGTDFTL |
| | | KISRVEAEDVGVYYCAQNLELPFTFGQGTKLEIK |
| 20 | HuEM1007- | DIVMTQTPLSLPVTPGEPASISCRSSKSLLYSNGITYLY |
| | mAb044VK.la | WYLQKPGQSPQLLIYQMSNLAPGVPDRFSSSGSGTDFTL |
| | | KISRVEAEDVGVYYCAQNLELPFTFGQGTKLEIK |
| 21 | HuEM1007- | DIVMTQTPLSLPVTPGEPASISCRSSKSLLYSNAITYLY |
| | mAb044VK.1b | WYLQKPGQSPQLLIYQMSNLAPGVPDRFSSSGSGTDFTL |
| | | KISRVEAEDVGVYYCAQNLELPFTFGQGTKLEIK |
| 22 | Anti-PD-L1 HCDR1 | TYGIN |
| 23 | Anti-PD-L1 HCDR2 | YIYIGNAYTEYNEKFKG |
| 24 | Anti-PD-L1 HCDR3 | DLMVIAPKTMDY |
| 25 | Anti-PD-L1 HCDR1 | YIYIGNGYTEYNEKFKG |
| 26 | Anti-PD-L1 LCDR1 | KASQDVGTAVA |
| 27 | Anti-PD-L1 LCDR2 | WASTRHT |
| 28 | Anti-PD-L1 LCDR3 | QQYSSYPYT |
| 29 | Anti-PD-L1 | QVQLQQSGAELVRPGSSVKMSCKTSGYTFTTYGINWVKQRPGQ |
| | EM0005-mAb86 | GLEWIGYIYIGNGYTEYNEKFKGKATLTSDPSSRTAYMQLSSL |
| | VH | TSEDSAIYFCARDLMVIAPKTMDYWGQGTSVTVSS |
| 30 | HuEM0005-86-21 VH | QVQLVQSGSELKKPGASVKVSCKASGYTFTTYGINWVRQ |
| | | APGQGLEWMGYIYIGNAYTEYNEKFKGRFVFSLDTSVST |
| | | AYLQICSLKAEDTAVYYCARDLMVIAPKTMDYWGQGTTV |
| | | TVSS |
| 31 | HuEM0005-86-64 VH | EVQLVQSGSELKKPGASVKVSCKASGYTFTTYGINWVRQ |
| | | APGQGLEWMGYIYIGNAYTEYNEKFKGRFVFSLDTSVST |
| | | AYLQISSLKAEDTAVYYCARDLMVIAPKTMDYWGQGTTV |
| | | TVSS |
| 32 | Anti-PD-L1 | DIQMNQSHKFMSTSVGDRVSITCKASQDVGTAVAWYQQKPGQS |
| | EM0005-mAb86 | PKLLIYWASTRHTGVPDRFTGGGSGTDFTLTISNVQSEDLADY |
| | VL | FCQQYSSYPYTFGGGTKLEMK |
| 33 | HuEM0005-86-21 VL | DIQMTQSPSSVSASVGDRVTITCKASQDVGTAVAWYQQK |
| | | PGKAPKLLIYWASTRHTGVPSRFSGSGSGTDFTLTISSL |
| | | QPEDFATYYCQQYSSYPYTFGGGTKVEIK |
| 34 | HuEM0005-86-64 VL | DIQMTQSPSSVSASVGDRVTITCKASQDVGTAVAWYQQK |
| | | PGKAPKLLIYWASTRHTGVPSRFSGSGSGTDFTLTISSL |
| | | QPEDFATYYCQQYSSYPYTFGGGTKVEIK |
| 35 | FIT1014-20a Chain #1 | DIQMTQSPSSVSASVGDRVTITCKASQDVGTAVAWYQQKPGKA |
| | VL_{PDL1}-CL-VH_{Ox40}- | PKLLIYWASTRHTGVPSRFSGSGSGTDFTLTISSLQPEDFATY |
| | CH1-Fc | YCQQYSSYPYTFGGGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGECE |
| | | VQLQQSGAEVKKPGSSVKVSCKASGHAFSSSWMNWVKQRPGKG |
| | | LEWIGRIYPGDEITNYNAKFKDKATLTADKSTSTAYMELSSLR |
| | | SEDTAVYYCARDLLMPYWGQGTLVTVSSASTKGPSVFPLAPSS |
| | | KSTSGGTAALGCLVKDYFPEPVTVSWNSGALTSGVHTFPAVLQ |
| | | SSGLYSLSSVVTVPSSSLGTQTYICNVNHKPSNTKVDKKVEPK |
| | | SCDKTHTCPPCPAPEAAGGPSVFLFPPKPKDTLYITREPEVTC |
| | | VVVDVSHEDPEVKFNWYVDGVEVHNAKTKPREEQYNSTYRVVS |
| | | VLTVLHQDWLNGKEYKCKVSNKALPAPIEKTISKAKGQPREPQ |
| | | VYTLPPSREEMTKNQVSLTCLVKGFYPSDIAVEWESNGQPENN |
| | | YKTTPPVLDSDGSFFLYSKLTVDKSRWQQGNVFSCSVMHEALH |
| | | NHYTQKSLSLSPGK |
| 36 | FIT1014-20a Chain #2 | EVQLVQSGSELKKPGASVKVSCKASGYTFTTYGINWVRQAPGQ |
| | VH_{PDL1}-CH1 | GLEWMGYIYIGNAYTEYNEKFKGRFVFSLDTSVSTAYLQISSL |
| | | KAEDTAVYYCARDLMVIAPKTMDYWGQGTTVTVSSASTKGPSV |
| | | FPLAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKKVEPKSC |
| 37 | FIT1014-20a Chain #3 | DIVMTQTPLSLPVTPGEPASISCRSSKSLLYSNAITYLYWYLQ |
| | VL_{OX40}-CL | KPGQSPQLLIYQMSNLAPGVPDRFSSSGSGTDFTLKISRVEAE |
| | | DVGVYYCAQNLELPFTFGQGTKLEIKRTVAAPSVFIFPPSDEQ |
| | | LKSGTASVVCLLNNFYPREAKVQWKVDNALQSGNSQESVTEQD |
| | | SKDSTYSLSSTLTLSKADYEKHKVYACEVTHQGLSSPVTKSFN |
| | | RGEC |
| 38 | VH_{OX40}-CH1 | EVQLQQSGAEVKKPGSSVKVSCKASGHAFSSSWMNWVKQ |
| | | RPGKGLEWIGRIYPGDEITNYNAKFKDKATLTADKSTST |
| | | AYMELSSLRSEDTAVYYCARDLLMPYWGQGTLVTVSSAS |
| | | TKGPSVFPLAPSSKSTSGGTAALGCLVKDYFPEPVTVSW |
| | | NSGALTSGVHTFPAVLQSSGLYSLSSVVTVPSSSLGTQT |
| | | YICNVNHKPSNTKVDKKVEPKSC |
| 39 | VL_{PDL1}-CL | DIQMTQSPSSVSASVGDRVTITCKASQDVGTAVAWYQQK |
| | | PGKAPKLLIYWASTRHTGVPSRFSGSGSGTDFTLTISSL |
| | | QPEDFATYYCQQYSSYPYTFGGGTKVEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 40 | Fc region from | DKTHTCPPCPAPEAAGGPSVFLFPPKPKDTLMISRTPEV |
| | hIgG1 with LALA | TCVVVDVSHEDPEVKFNWYVDGVEVHNAKTKPREEQYNS |
| | mutations | TYRVVSVLTVLHQDWLNGKEYKCKVSNKALPAPIEKTIS |
| | | KAKGQPREPQVYTLPPSREEMTKNQVSLTCLVKGFYPSD |
| | | IAVEWESNGQPENNYKTTPPVLDSDGSFFLYSKLTVDKS |
| | | RWQQGNVFSCSVMHEALHNHYTQKSLSLSPGK |
| 41 | Fc region from | DKTHTCPPCPAPEAAGGPSVFLFPPKPKDTLYITREPEV |
| | hIgG1 with | TCVVVDVSHEDPEVKFNWYVDGVEVHNAKTKPREEQYNS |
| | mutations | TYRVVSVLTVLHQDWLNGKEYKCKVSNKALPAPIEKTIS |
| | LALA&YTE | KAKGQPREPQVYTLPPSREEMTKNQVSLTCLVKGFYPSD |
| | | IAVEWESNGQPENNYKTTPPVLDSDGSFFLYSKLTVDKS |
| | | RWQQGNVFSCSVMHEALHNHYTQKSLSLSPGK |
| 42 | Heavy chain | ASTKGPSVFPLAPSSKSTSGGTAALGCLVKDYFPEPVTV |
| | constant domain | SWNSGALTSGVHTFPAVLQSSGLYSLSSVVTVPSSSLGT |
| | from hIgG1 with | QTYICNVNHKPSNTKVDKKVEPKSCDKTHTCPPCPAPEA |
| | mutations LALA | AGGPSVFLFPPKPKDTLMISRTPEVTCVVVDVSHEDPEV |
| | | KFNWYVDGVEVHNAKTKPREEQYNSTYRVVSVLTVLHQD |
| | | WLNGKEYKCKVSNKALPAPIEKTISKAKGQPREPQVYTL |
| | | PPSREEMTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| 43 | human kappa light | RTVAAPSVFIFPPSDEQLKSGTASVVCLLNNFYPREAKV |
| | chain constant | QWKVDNALQSGNSQESVTEQDSKDSTYSLSSTLTLSKAD |
| | domain | YEKHKVYACEVTHQGLSSPVTKSFNRGEC |
| 44 | Human OX40 | MCVGARRLGRGPCAALLLLGLGLSTVTGLHCVGDTYPSN |
| | | DRCCHECRPGNGMVSRCSRSQNTVCRPCGPGFYNDVVSS |
| | | KPCKPCTWCNLRSGSERKQLCTATQDTVCRCRAGTQPLD |
| | | SYKPGVDCAPCPPGHFSPGDNQACKPWTNCTLAGKHTLQ |
| | | PASNSSDAICEDRDPPATQPQETQGPPARPITVQPTEAW |
| | | PRTSQGPSTRPVEVPGGRAVAAILGLGLVLGLLGPLAIL |
| | | LALYLLRRDQRLPPDAHKPPGGGSFRTPIQEEQADAHST |
| | | LAKI |

### REFERENCE

Aspeslagh S. et al., Eur. J. Cancer. (2016). Rationale for anti-OX40 cancer immunotherapy. 52:50-66.
Brahmer J R. et al., N. Engl. J. Med (2012). Safety and activity of anti-PD-L1 antibody in patients with advanced cancer. 366(26): 2455-65.
Bulliard Y. et al., Immunol Cell Biol. (2014). OX40 engagement depletes intratumoral Tregs via activating FcγRs, leading to antitumor efficacy. 92(6): 475-80.
Choi Y. et al., J Immunother Cancer. (2020). T-cell agonists in cancer immunotherapy.
Carolina Alves Costa Silva. et al., ESMO Open (2020). New pathways in immune stimulation: targeting OX40. 5(1): e000573.
Croft M., Annu. Rev. Immunol. (2010). Control of immunity by the TNFR-related molecule OX40 (CD134). 28:57-78.
Curti B D. et al., Cancer Res. (2013). OX40 is a potent immune-stimulating target in late-stage cancer patients. 73(24): 7189-7198
Gieffers C. et al., Mol Cancer Ther (2013). APG350 induces superior clustering of TRAIL receptors and shows therapeutic antitumor efficacy independent of cross-linking via Fcγ receptors. 12(12): 2735-47.
Glisson B S. et al., Clin Cancer Res. 2020. Safety and Clinical Activity of MEDI0562, a Humanized OX40 Agonist Monoclonal Antibody, in Adult Patients with Advanced Solid Tumors. 26(20): 5358-5367.
Gutierrez M. et al., Clin Cancer Res. (2021). OX40 Agonist BMS-986178 Alone or in Combination with Nivolumab and/or Ipilimumab in Patients with Advanced Solid Tumors. 27(2): 460-472.
Jacquemin C. et al., Immunity. (2015). OX40 Ligand Contributes to Human Lupus Pathogenesis by Promoting T Follicular Helper Response. 42(6): 1159-70.
Kawamata S. et al., J. Biol. Chem (1998). Activation of OX40 signal transduction pathways leads to tumor necrosis factor receptor-associated factor (TRAF) 2-andTRAF5-mediated NF-kappaB activation. 273 (10), 5808-5814.
Mayes P A, Hance K W, Hoos A. Nat Rev Drug Discov. (2018). The promise and challenges of immune agonist antibody development in cancer. 17 (7): 509-527.
Schaer D A. Et al., J. Immunother. Cancer (2014) Targeting tumor-necrosis factor receptor pathways for tumor immunotherapy. 2, 7.
Smyth M J. et al., Immunol Cell Biol. (2014). Targeting regulatory T cells in tumor immunotherapy. 92 (6): 473-4.
Song J. et al., Immunol (2008). Activation of NF-kappaB1 by OX40 contributes to antigen-driven T cell expansion and survival. 180 (11), 7240-7248.
Tourkova I L. et al., Immunol Lett. (2001). Mechanisms of dendritic cell-induced T cell proliferation in the primary MLR assay. 78(2): 75-82.
Watts T H. Annu. Rev. Immunol. (2005). TNF/TNFR family members in costimulation of T cell responses. Annu. Rev. Immunol. 23, 23-68.
Weinberg A D. et al., Immunol Rev. (2011). Science gone translational: the OX40 agonist story. 2011, 244:218-231
Willoughby J. et al., Mol Immunol (2017). March; 83:13-22.
Zhang X, et al., Cell Rep. (2018). OX40 Costimulation Inhibits Foxp3 Expression and Treg Induction via BATF3-Dependent and Independent Mechanisms. 24(3): 607-618.

1. An isolated antibody or antigen-binding fragment thereof that specifically binds to OX40, comprising a set of six CDRs, CDR-H1, CDR-H2, CDR-H3, CDR-L1, CDR-L2, and CDR-L3, wherein:
the CDR-H1 comprises the sequence of SSWMN (SEQ ID NO:1);
the CDR-H2 comprises the sequence of RIYPGDEITNYNGKFKD (SEQ ID NO: 2) or RIYPGDEITNYNAKFKD (SEQ ID NO: 4);
the CDR-H3 comprises the sequence of DLLMPY (SEQ ID NO: 3);
the CDR-L1 comprises the sequence of RSSKSLLYSNGITYLY (SEQ ID NO: 5) or RSSKSLLYSNAITYLY (SEQ ID NO: 8);
the CDR-L2 comprises the sequence of QMSNLAP (SEQ ID NO: 6); and
the CDR-L3 comprises the sequence of AQNLELPFT (SEQ ID NO: 7),
optionally wherein the CDRs are defined according to Kabat numbering.

2. The isolated antibody or antigen-binding fragment of claim 1, wherein
(a) the antibody has one or more of the following characteristics:
(i) upon binding to the cell surface of OX40-expressing cells (e.g. OX40-expressing T cells), displays strong binding potency to OX40+ cells, wherein said cell binding potency is reflected by an EC50 of about 5 nM or lower, 4 nM or lower, 3 nM or lower, 2 nM or lower, or 1 nM or lower, as measured by flow cytometry in a cell-based assay;
(ii) the antibody binds to human OX40 at CRD3 of the OX40's extracellular domain; or
(iii) binding of the antibody to OX40 induces anti-tumor immunity of T cells, e.g., reduced tumor burden/growth/cell expansion, optionally wherein said anti-tumor immunity comprises anti-tumor cytotoxicity and secretion of anti-tumor cytokines,
(b) the antibody comprises a variable heavy chain domain VH and a variable light chain domain VL, wherein:
the VH domain comprises the sequence of SEQ ID NO:9 or 10, or a sequence having at least 80%, 85%, 90%, 91%, 92%, 93%, 94%, 95%, 96%, 97%, 98%, 99% or more identity therewith, and/or the VL domain comprises the sequence of SEQ ID NO: 17 or 18, or a sequence having at least 80%, 85%, 90%, 91%, 92%, 93%, 94%, 95%, 96%, 97%, 98%, 99% or more identity therewith;
or
the VH domain comprises the sequence selected from any one of SEQ ID NOs: 11-16, or a sequence having at least 80%, 85%, 90%, 91%, 92%, 93%, 94%, 95%, 96%, 97%, 98%, 99% or more identity therewith, and/or the VL domain comprises the sequence selected from any one of SEQ ID NOs: 19-21, or a sequence having at least 80%, 85%, 90%, 91%, 92%, 93%, 94%, 95%, 96%, 97%, 98%, 99% or more identity therewith,
(c) the VH domain of the antibody comprises amino acid residues 1E, and 1 to 10 residues selected from 5Q, 27H, 28A, 38K, 40R, 43K, 48I, 67K, 68A, 70L, e.g., 10 residues, according to Kabat numbering; and the VL domain comprises amino acid residue 69G or 69S, e.g., 69S, according to Kabat numbering, or
(d) the antibody comprises a combination of VH and VL sequences selected from the group consisting of:

| combination | VH sequence | VL sequence |
|---|---|---|
| 1 | SEQ ID NO: 11 | SEQ ID NO: 19 |
| 2 | SEQ ID NO: 12 | SEQ ID NO: 19 |
| 3 | SEQ ID NO: 13 | SEQ ID NO: 19 |
| 4 | SEQ ID NO: 14 | SEQ ID NO: 19 |
| 5 | SEQ ID NO: 11 | SEQ ID NO: 20 |
| 6 | SEQ ID NO: 12 | SEQ ID NO: 20 |
| 7 | SEQ ID NO: 13 | SEQ ID NO: 20 |
| 8 | SEQ ID NO: 14 | SEQ ID NO: 20 |
| 9 | SEQ ID NO: 10 | SEQ ID NO: 17 |
| 10 | SEQ ID NO: 9 | SEQ ID NO: 18 |
| 11 | SEQ ID NO: 10 | SEQ ID NO: 18 |
| 12 | SEQ ID NO: 9 | SEQ ID NO: 19 |
| 13 | SEQ ID NO: 11 | SEQ ID NO: 17 |
| 14 | SEQ ID NO: 15 | SEQ ID NO: 21 |
| 15 | SEQ ID NO: 15 | SEQ ID NO: 18 |
| 16 | SEQ ID NO: 16 | SEQ ID NO: 21 |
| 17 | SEQ ID NO: 16 | SEQ ID NO: 18 |

optionally wherein the antibody comprises a VH domain comprising the sequence of SEQ ID NO: 16 and a VL domain comprising the sequence of SEQ ID NO: 21.

3-5. (canceled)

6. The isolated antibody or antigen-binding fragment of claim 1, wherein the antibody comprises an Fc region having the amino acid sequence of SEQ ID NO: 40.

7. A fusion or a conjugate comprising the isolated antibody or antigen-binding fragment of claim 1.

8. An isolated antibody or antigen-binding fragment thereof that specifically binds to PD-L1, comprising a set of six CDRs, CDR-H1, CDR-H2, CDR-H3, CDR-L1, CDR-L2, and CDR-L3, wherein:
the CDR-H1 comprises the sequence of TYGIN (SEQ ID NO:22);
the CDR-H2 comprises the sequence of YIYIGNAYTEYNEKFKG (SEQ ID NO: 23) or YIYIGNGYTEYNEKFKG (SEQ ID NO: 25);
the CDR-H3 comprises the sequence of DLMVIAPKTMDY (SEQ ID NO: 24);
the CDR-L1 comprises the sequence of KASQDVGTAVA (SEQ ID NO: 26);
the CDR-L2 comprises the sequence of WASTRHT (SEQ ID NO: 27); and
the CDR-L3 comprises the sequence of QQYSSYPYT (SEQ ID NO: 28),
optionally wherein the CDRs are defined according to Kabat numbering.

9. The isolated antibody or antigen-binding fragment of claim 8, wherein the antibody comprises a variable heavy chain domain VH and a variable light chain domain VL, wherein:
the VH domain comprises the sequence of SEQ ID NO:29, or a sequence having at least 80%, 85%, 90%, 91%, 92%, 93%, 94%, 95%, 96%, 97%, 98%, 99% or more identity therewith, and/or the VL domain comprises the sequence of SEQ ID NO:32, or a sequence having at least 80%, 85%, 90%, 91%, 92%, 93%, 94%, 95%, 96%, 97%, 98%, 99% or more identity therewith;
or
the VH domain comprises the sequence selected from any one of SEQ ID NO: 30 or 31, or a sequence having at least 80%, 85%, 90%, 91%, 92%, 93%, 94%, 95%, 96%, 97%, 98%, 99% or more identity therewith, and/or the VL domain comprises the sequence selected from any one of SEQ ID NOs: 33 or 34, or a sequence having at least 80%, 85%, 90%, 91%, 92%, 93%, 94%, 95%, 96%, 97%, 98%, 99% or more identity therewith.

10. (canceled)

11. The isolated antibody or antigen-binding fragment of claim 8, wherein the antibody comprises an Fc region having the amino acid sequence of SEQ ID NO: 40.

12. A fusion or a conjugate comprising the isolated antibody or antigen-binding fragment of claim 8.

13. A nucleic acid molecule encoding the isolated antibody or antigen-binding fragment of claim 1.

14. (canceled)

15. A host cell expressing the nucleic acid molecule encoding the isolated antibody or antigen-binding fragment of claim 1.

16. A pharmaceutical composition comprising the isolated antibody or antigen-binding fragment of claim 1.

17. A method of detecting OX40 in a biological sample, comprising contacting the biological sample with the isolated antibody or antigen-binding fragment of claim 1.

18. (canceled)

19. A bispecific binding protein that specifically binds OX40 and PD-L1, comprising a first antigen-binding site that specifically binds OX40, and a second antigen-binding site that specifically binds PD-L1, wherein
the first antigen-binding site comprises a set of six CDRs, CDR-H1, CDR-H2, CDR-H3, CDR-L1, CDR-L2, and CDR-L3, wherein:
the CDR-H1 comprises the sequence of SSWMN (SEQ ID NO:1),
the CDR-H2 comprises the sequence of RIYPGDEITNYNGKFKD (SEQ ID NO: 2) or RIYPGDEITNYNAKFKD (SEQ ID NO: 4),
the CDR-H3 comprises the sequence of DLLMPY (SEQ ID NO: 3),
the CDR-L1 comprises the sequence of RSSKSLLYSNGITYLY (SEQ ID NO: 5) or RSSKSLLYSNAITYLY (SEQ ID NO: 8),
the CDR-L2 comprises the sequence of QMSNLAP (SEQ ID NO: 6), and
the CDR-L3 comprises the sequence of AQNLELPFT (SEQ ID NO: 7),
and/or the second antigen-binding site comprises a set of six CDRs, CDR-H1, CDR-H2, CDR-H3, CDR-L1, CDR-L2, and CDR-L3, wherein:
the CDR-H1 comprises the sequence of TYGIN (SEQ ID NO:22),
the CDR-H2 comprises the sequence of YIYIGNAYTEYNEKFKG (SEQ ID NO: 23) or YIYIGNGYTEYNEKFKG (SEQ ID NO: 25),
the CDR-H3 comprises the sequence of DLMVIAPKTMDY (SEQ ID NO: 24),
the CDR-L1 comprises the sequence of KASQDVGTAVA (SEQ ID NO: 26),
the CDR-L2 comprises the sequence of WASTRHT (SEQ ID NO: 27), and
the CDR-L3 comprises the sequence of QQYSSYPYT (SEQ ID NO: 28),
optionally, the second antigen-binding site comprises a VH domain comprising the sequence of SEQ ID NO: 31, or a sequence having at least 80%, 85%, 90%, 91%, 92%, 93%, 94%, 95%, 96%, 97%, 98%, 99% or more identity therewith, and/or a VL domain comprising the sequence of SEQ ID NO: 34, or a sequence having at least 80%, 85%, 90%, 91%, 92%, 93%, 94%, 95%, 96%, 97%, 98%, 99% or more identity therewith;
wherein the CDRs are defined according to Kabat numbering.

20. The bispecific binding protein of claim 19, comprising a first polypeptide chain, a second polypeptide chain and a third polypeptide chain,
wherein
(i) the first polypeptide chain comprises, from amino terminus to carboxyl terminus, VL_{A}-CL-VH_{B}-CH1-Fc with CL fused directly to VH_{B} or VH_{B}—CH1-VL_{A}-CL-Fc with CH1 fused directly to VL_{A}; the second polypeptide chain comprises, from amino to carboxyl terminus, VH_{A}-CH1; the third polypeptide chain comprises, from amino to carboxyl terminus, VL_{B}-CL; or
(ii) the first polypeptide chain comprises, from amino terminus to carboxyl terminus, VH_{A}-CH1-VL_{B}-CL-Fc with CH1 fused directly to VL_{B} or VL_{B}-CL-VH_{A}-CH1-Fc with CL fused directly to VH_{A}; the second polypeptide chain comprises, from amino to carboxyl terminus, VH_{B}-CH1; the third polypeptide chain comprises, from amino to carboxyl terminus, VL_{A}-CL;
wherein VL is a light chain variable domain, CL is a light chain constant domain, VH is a heavy chain variable domain, CH1 is a heavy chain constant domain, Fc is an immunoglobulin Fc region, for example, the Fc of IgG1 (optionally, comprising, from amino terminus to carboxyl terminus, hinge-CH2-CH3),
wherein the VL_{A}-CL pairs with VH_{A}-CH1 to form a first Fab that specifically binds a first antigen A, and VL_{B}-CL pairs with VH_{B}-CH1 to form a second Fab that specifically binds a second antigen B, and
wherein the first antigen A is OX40, and the second antigen B is PD-L1,
wherein two of the first polypeptide chains, two of the second polypeptide chains, and two of the third polypeptide chains are associated to form a FIT-Ig protein.

21. The bispecific binding protein of claim 19, wherein:
the first polypeptide chain comprises an amino acid sequence of SEQ ID NO:35, or a sequence having at least 80%, 85%, 90%, 91%, 92%, 93%, 94%, 95%, 96%, 97%, 98%, 99% or more identity therewith,
the second polypeptide chain comprises an amino acid sequence of SEQ ID NO:36, or a sequence having at least 80%, 85%, 90%, 91%, 92%, 93%, 94%, 95%, 96%, 97%, 98%, 99% or more identity therewith, and
the third polypeptide chain comprises an amino acid sequence of SEQ ID NO:37, or a sequence having at least 80%, 85%, 90%, 91%, 92%, 93%, 94%, 95%, 96%, 97%, 98%, 99% or more identity therewith.

22. (canceled)

23. A nucleic acid molecule encoding the bispecific binding protein of claim 19.

24. (canceled)

25. A host cell comprising the nucleic acid molecule of claim 23.

26. A method of preparing the isolated antibody or antigen-binding fragment of claim 1, comprising:
culturing a host cell comprising a nucleic acid molecule encoding the antibody or antigen-binding fragment thereof under conditions that allow the production of the antibody or antigen-binding fragment; and
recovering the antibody or antigen-binding fragment from the culture.

27. (canceled)

28. A method of treating a disorder wherein OX40-mediated activity is advantageous, comprising administering to a subject in need thereof a therapeutically effective amount of the pharmaceutical composition of claim 16, wherein the disorder is optionally cancer, preferably colon cancer.

29-30. (canceled)

31. A method of treating a disorder wherein PD-L1-associated activity is detrimental, comprising administering to a subject in need thereof a therapeutically effective amount of the isolated antibody or antigen-binding fragment of claim 8, wherein the disorder is optionally cancer, preferably colon cancer.


SEQUENCE LISTING (44 sequences)

SEQ ID NO: 1 - protein, 5 aa, Artificial Sequence
  Anti-OX40 HCDR1
SSWMN

SEQ ID NO: 2 - protein, 17 aa, Artificial Sequence
  Anti-OX40 HCDR2
RIYPGDEITNYNGKFKD

SEQ ID NO: 3 - protein, 6 aa, Artificial Sequence
  Anti-OX40 HCDR3
DLLMPY

SEQ ID NO: 4 - protein, 17 aa, Artificial Sequence
  Anti-OX40 HCDR2
RIYPGDEITNYNAKFKD

SEQ ID NO: 5 - protein, 16 aa, Artificial Sequence
  Anti-OX40 LCDR1
RSSKSLLYSNGITYLY

SEQ ID NO: 6 - protein, 7 aa, Artificial Sequence
  Anti-OX40 LCDR2
QMSNLAP

SEQ ID NO: 7 - protein, 9 aa, Artificial Sequence
  Anti-OX40 LCDR3
AQNLELPFT

SEQ ID NO: 8 - protein, 16 aa, Artificial Sequence
  Anti-OX40 LCDR1
RSSKSLLYSNAITYLY

SEQ ID NO: 9 - protein, 115 aa, Artificial Sequence
  EM1007-mAb044VH (clone.8G9D5C5)
QVQLQQSGPELVKPGASVTISCKASGHAFSSSWMNWVKQRPGKGLEWIGRIYPGDEITNYNGKFKDKATLTADKSSSTAYMQLSSLTFEDSAVYFCARDLLMPYWGQGTLVTVSA

SEQ ID NO: 10 - protein, 115 aa, Artificial Sequence
  EM1007-mAb044VH(G-A)
QVQLQQSGPELVKPGASVTISCKASGHAFSSSWMNWVKQRPGKGLEWIGRIYPGDEITNYNAKFKDKATLTADKSSSTAYMQLSSLTFEDSAVYFCARDLLMPYWGQGTLVTVSA

SEQ ID NO: 11 - protein, 115 aa, Artificial Sequence
  HuEM1007-mAb044VH.1
EVQLVQSGAEVKKPGSSVKVSCKASGGTFSSSWMNWVRQAPGQGLEWMGRIYPGDEITNYNGKFKDRVTITADKSTSTAYMELSSLRSEDTAVYYCARDLLMPYWGQGTLVTVSS

SEQ ID NO: 12 - protein, 115 aa, Artificial Sequence
  HuEM1007-mAb044VH.1a
EVQLVQSGAEVKKPGSSVKVSCKASGHTFSSSWMNWVRQAPGQGLEWMGRIYPGDEITNYNGKFKDRVTITADKSTSTAYMELSSLRSEDTAVYYCARDLLMPYWGQGTLVTVSS

SEQ ID NO: 13 - protein, 115 aa, Artificial Sequence
  HuEM1007-mAb044VH.1b
EVQLVQSGAEVKKPGSSVKVSCKASGHTFSSSWMNWVRQAPGQGLEWIGRIYPGDEITNYNGKFKDRVTLTADKSTSTAYMELSSLRSEDTAVYYCARDLLMPYWGQGTLVTVSS

SEQ ID NO: 14 - protein, 115 aa, Artificial Sequence
  HuEM1007-mAb044VH.1c
EVQLVQSGAEVKKPGSSVKVSCKASGHTFSSSWMNWVKQAPGKGLEWIGRIYPGDEITNYNGKFKDKVTLTADKSTSTAYMELSSLRSEDTAVYYCARDLLMPYWGQGTLVTVSS

SEQ ID NO: 15 - protein, 115 aa, Artificial Sequence
  HuEM1007-mAb044VH.1d
EVQLVQSGAEVKKPGSSVKVSCKASGGTFSSSWMNWVRQRPGKGLEWMGRIYPGDEITNYNAKFKDRVTITADKSTSTAYMELSSLRSEDTAVYYCARDLLMPYWGQGTLVTVSS

SEQ ID NO: 16 - protein, 115 aa, Artificial Sequence
  HuEM1007-mAb044VH.1e
EVQLQQSGAEVKKPGSSVKVSCKASGHAFSSSWMNWVKQRPGKGLEWIGRIYPGDEITNYNAKFKDKATLTADKSTSTAYMELSSLRSEDTAVYYCARDLLMPYWGQGTLVTVSS

SEQ ID NO: 17 - protein, 112 aa, Artificial Sequence
  EM1007-mAb044VK
DIVMTQTAFSNPVTLGTSASISCRSSKSLLYSNGITYLYWYLQKPGQSPHLLIYQMSNLAPGVPDRFSSSGSGTDFTLRISRVEAEDVGIYYCAQNLELPFTFGSGTKLEIK

SEQ ID NO: 18 - protein, 112 aa, Artificial Sequence
  EM1007-mAb044VK(G-A)
DIVMTQTAFSNPVTLGTSASISCRSSKSLLYSNAITYLYWYLQKPGQSPHLLIYQMSNLAPGVPDRFSSSGSGTDFTLRISRVEAEDVGIYYCAQNLELPFTFGSGTKLEIK

SEQ ID NO: 19 - protein, 112 aa, Artificial Sequence
  HuEM1007-mAb044VK.1
DIVMTQTPLSLPVTPGEPASISCRSSKSLLYSNGITYLYWYLQKPGQSPQLLIYQMSNLAPGVPDRFSGSGSGTDFTLKISRVEAEDVGVYYCAQNLELPFTFGQGTKLEIK

SEQ ID NO: 20 - protein, 112 aa, Artificial Sequence
  HuEM1007-mAb044VK.1a
DIVMTQTPLSLPVTPGEPASISCRSSKSLLYSNGITYLYWYLQKPGQSPQLLIYQMSNLAPGVPDRFSSSGSGTDFTLKISRVEAEDVGVYYCAQNLELPFTFGQGTKLEIK

SEQ ID NO: 21 - protein, 112 aa, Artificial Sequence
  HuEM1007-mAb044VK.1b
DIVMTQTPLSLPVTPGEPASISCRSSKSLLYSNAITYLYWYLQKPGQSPQLLIYQMSNLAPGVPDRFSSSGSGTDFTLKISRVEAEDVGVYYCAQNLELPFTFGQGTKLEIK

SEQ ID NO: 22 - protein, 5 aa, Artificial Sequence
  Anti-PD-L1 HCDR1
TYGIN

SEQ ID NO: 23 - protein, 17 aa, Artificial Sequence
  Anti-PD-L1 HCDR2
YIYIGNAYTEYNEKFKG

SEQ ID NO: 24 - protein, 12 aa, Artificial Sequence
  Anti-PD-L1 HCDR3
DLMVIAPKTMDY

SEQ ID NO: 25 - protein, 17 aa, Artificial Sequence
  Anti-PD-L1 HCDR1
YIYIGNGYTEYNEKFKG

SEQ ID NO: 26 - protein, 11 aa, Artificial Sequence
  Anti-PD-L1 LCDR2
KASQDVGTAVA

SEQ ID NO: 27 - protein, 7 aa, Artificial Sequence
  Anti-PD-L1 LCDR2
WASTRHT

SEQ ID NO: 28 - protein, 9 aa, Artificial Sequence
  Anti-PD-L1 LCDR3
QQYSSYPYT

SEQ ID NO: 29 - protein, 121 aa, Artificial Sequence
  Anti-PD-L1 EM0005-mAb86 VH
QVQLQQSGAELVRPGSSVKMSCKTSGYTFTTYGINWVKQRPGQGLEWIGYIYIGNGYTEYNEKFKGKATLTSDPSSRTAYMQLSSLTSEDSAIYFCARDLMVIAPKTMDYWGQGTSVTVSS

SEQ ID NO: 30 - protein, 121 aa, Artificial Sequence
  HuEM0005-86-21 VH
QVQLVQSGSELKKPGASVKVSCKASGYTFTTYGINWVRQAPGQGLEWMGYIYIGNAYTEYNEKFKGRFVFSLDTSVSTAYLQICSLKAEDTAVYYCARDLMVIAPKTMDYWGQGTTVTVSS

SEQ ID NO: 31 - protein, 121 aa, Artificial Sequence
  HuEM0005-86-64 VH
EVQLVQSGSELKKPGASVKVSCKASGYTFTTYGINWVRQAPGQGLEWMGYIYIGNAYTEYNEKFKGRFVFSLDTSVSTAYLQISSLKAEDTAVYYCARDLMVIAPKTMDYWGQGTTVTVSS

SEQ ID NO: 32 - protein, 107 aa, Artificial Sequence
  Anti-PD-L1 EM0005-mAb86 VL
DIQMNQSHKFMSTSVGDRVSITCKASQDVGTAVAWYQQKPGQSPKLLIYWASTRHTGVPDRFTGGGSGTDFTLTISNVQSEDLADYFCQQYSSYPYTFGGGTKLEMK

SEQ ID NO: 33 - protein, 107 aa, Artificial Sequence
  HuEM0005-86-21 VL
DIQMTQSPSSVSASVGDRVTITCKASQDVGTAVAWYQQKPGKAPKLLIYWASTRHTGVPSRFSGSGSGTDFTLTISSLQPEDFATYYCQQYSSYPYTFGGGTKVEIK

SEQ ID NO: 34 - protein, 107 aa, Artificial Sequence; identical to SEQ ID NO: 33
  HuEM0005-86-64 VL

SEQ ID NO: 35 - protein, 659 aa, Artificial Sequence
  FIT1014-20a Chain #1 VLPDL1-CL-VHOX40-CH1-Fc
DIQMTQSPSSVSASVGDRVTITCKASQDVGTAVAWYQQKPGKAPKLLIYWASTRHTGVPSRFSGSGSGTDFTLTISSLQPEDFATYYCQQYSSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGAEVKKPGSSVKVSCKASGHAFSSSWMNWVKQRPGKGLEWIGRIYPGDEITNYNAKFKDKATLTADKSTSTAYMELSSLRSEDTAVYYCARDLLMPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 224 aa, Artificial Sequence
  FIT1014-20a Chain #2 VHPDL1-CH1
EVQLVQSGSELKKPGASVKVSCKASGYTFTTYGINWVRQAPGQGLEWMGYIYIGNAYTEYNEKFKGRFVFSLDTSVSTAYLQISSLKAEDTAVYYCARDLMVIAPKTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 37 - protein, 219 aa, Artificial Sequence
  FIT1014-20a Chain #3 VLOX40-CL
DIVMTQTPLSLPVTPGEPASISCRSSKSLLYSNAITYLYWYLQKPGQSPQLLIYQMSNLAPGVPDRFSSSGSGTDFTLKISRVEAEDVGVYYCAQNLELPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 218 aa, Artificial Sequence
  VHOX40-CH1
EVQLQQSGAEVKKPGSSVKVSCKASGHAFSSSWMNWVKQRPGKGLEWIGRIYPGDEITNYNAKFKDKATLTADKSTSTAYMELSSLRSEDTAVYYCARDLLMPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 39 - protein, 214 aa, Artificial Sequence
  VLPDL1-CL
DIQMTQSPSSVSASVGDRVTITCKASQDVGTAVAWYQQKPGKAPKLLIYWASTRHTGVPSRFSGSGSGTDFTLTISSLQPEDFATYYCQQYSSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 227 aa, Artificial Sequence
  Fc region from hIgG1 with LALA mutations
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 227 aa, Artificial Sequence
  Fc region from hIgG1 with mutations LALA&YTE
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 330 aa, Artificial Sequence
  Heavy chain constant domain from hIgG1 with mutations LALA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 107 aa, Artificial Sequence [sequence omitted]

SEQ ID NO: 44 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI